(19) United States
(12) Patent Application Publication
(10) Pub. No.: US 2005/0267159 A1
(43) Pub. Date: Dec. 1, 2005

(54) Magnesium complexes of S-omeprazole

(75) Inventors: David White, Chicago, IL (US); Robert R. Whittle, Wilmington, NC (US); Grayson W. Stowell, Wilmington, NC (US); Linda B. Whittall, Wilmington, NC (US)

Correspondence Address: MYERS BIGEL SIBLEY & SAJOVEC, PO BOX 37428, RALEIGH, NC, 27627, US

(73) Assignee: aaiPharma, Inc.

(21) Appl. No.: 11/138,195

(22) Filed: May 26, 2005

Related U.S. Application Data
(63) Continuation-in-part of application No. 10/855,809, filed on May 28, 2004.

(51) Int. Cl. A61K 31/4439; C07F 3/02; C07D 43/02

(52) U.S. Cl. 514/338; 546/273.7

(57) ABSTRACT

The invention provides magnesium S-omeprazolato coordination compounds according to formula (I):
[Mg(solvₐ)ₓ(solv_{b})_{y}][Mg(S-omeprazolato)₃]₂.(solv_{c})_{z} (I),
pharmaceutical compositions and processes of making the same. In formula (I), solvₐ, solv_{b}, and solv_{c} represent solvent molecules where x and y are independently selected from integers 0 to 6, the sum of which is 4 or 6, while z is a positive rational number from 0 to 6. The compounds are useful for the treatment of gastric acid related conditions and the inhibition of gastric acid secretion.

### CROSS-REFERENCE TO RELATED APPLICATION

This application claims priority from U.S. patent application Ser. No. 10/855,809, filed May 28, 2004, the disclosure of which is incorporated by reference herein in its entirety.

### BACKGROUND OF THE INVENTION

The present invention relates generally to the field of pharmaceutical agents that are effective as inhibitors of gastric acid secretion. In particular, the invention relates to magnesium coordination complexes of omeprazole and to their pharmaceutical compositions, processes of preparation, and uses.

Various compounds used in inhibiting gastric acid secretion are known in the art and include, in particular, a class of benzimidazole-substituted compounds, one of which is omeprazole. Omeprazole generally refers to rac-5-methoxy-2-{[(4-methoxy-3,5-dimethylpyridin-2-yl)methyl]sulfinyl}-1H-benzimidazole, rac-6-methoxy-2-{[(4-methoxy-3,5-dimethylpyridin-2-yl)methyl]sulfinyl}-1H-benzimidazole and mixtures thereof. It is currently commercially available, in a specific ratio range of 5-methoxy to 6-methoxy isomers in the solid state, in the formulation Prilosec®. U.S. Pat. No. 4,255,431, for example, contemplates such benzimidazole-substituted compounds, their pharmaceutical salts, and optical isomers thereof.

More recent developments in the art pertain to optically pure isomers of omeprazole, specifically S-omeprazole, and its related pharmaceutical salts. Certain disclosures ascribe particularly efficacious pharmaceutical activity to a magnesium salt of S-omeprazole, such as that purportedly contained in the commercial formulation Nexium®. For example, U.S. Pat. No. 5,714,504 to Lindberg et al. discloses a pharmaceutical formulation that comprises a pure solid state alkaline salt of the (−)-enantiomer of 5-methoxy-2-{[(4-methoxy-3,5-dimethylpyridin-2-yl)methyl]sulfinyl}-1H-benzimidazole. The '504 patent discloses in this regard certain optically pure magnesium salts of S-omeprazole and processes of making the same.

U.S. Pat. No. 6,369,085 to Cotton et al. discloses a highly crystalline form of a trihydrate of a magnesium S-omeprazole salt. The '085 patent ascribes certain X-ray powder diffractograms to the salt, thereby purportedly distinguishing it from other crystalline forms of the magnesium S-omeprazole salt. By contrast, WO 04/02982 discloses amorphous forms of the magnesium S-omeprazole salt di- and trihydrates.

These conventional teachings pertaining to the methods of making omeprazole, the purported salts and/or enantiomers thereof, together with formulations that may include these compounds, assume accurate determinations of the chemical structure of omeprazole, its optically pure isomers, and purported salts thereof. For example, as explained in U.S. Pat. No. 6,444,689 to Whittle et al., a methoxy group on the benzimidazole ring of omeprazole, an optically pure isomer, or racemic mixture thereof is stated in the literature to be present at the 5-position. It is now known that the methods of the prior art do not yield a single crystalline compound having the methoxy group in the 5-position on the benzimidazole ring, nor do all conventional methods yield consistent results. In this regard, omeprazole as conventionally referred to as a bulk drug substance or active pharmaceutical ingredient (i.e., in its solid state) has been discovered to exist in the form of two pharmaceutically active compounds having the methoxy group on the benzimidazole ring at the 6- and 5-positions. Additionally, the '689 patent discloses the presence of a second chiral location at the pyridine ring plane in each of the two compounds such that each compound has two positional isomers and four diastereomers.

As noted above, the state of the art implicates primarily X-ray powder diffractograms to characterize the purported magnesium salts of S-omeprazole in the cases where crystalline material can be obtained. A potential limitation of relying upon such data, however, is the inherent insensitivity of powder X-ray diffraction to different isostructural compounds generally, and to clathrates in particular. In this context, it is well-known that pharmaceutical compounds may give rise to similar or nearly identical powder diffractograms, despite the presence of different solvent molecules in various solid-state forms of the compounds. These features are significant because the properties of different forms of a pharmaceutical compound can influence its manufacturing process, dissolution rate, storage stability, and bioavailability. There remains therefore a need in the art to correctly identify and predictably manufacture magnesium compounds of omeprazole, its optically pure isomers, and solvates and combinations thereof.

### SUMMARY OF THE INVENTION

The present invention satisfies this need and other needs by providing a magnesium S-omeprazolato coordination compound in the solid state according to formula (I):
[Mg(solvₐ)ₓ(solv_{b})_{y}][Mg(S-omeprazolato)₃]₂.(solv_{c})_{z} (I),
wherein solvₐ is a coordinated solvent molecule that is selected from the group consisting of H₂O; ROH; ROR; RC(O)OR; RC(O)R; RC(S)R; RS(O)R; R₂NC(O)R; and an optionally substituted 5- or 6-membered heterocyclic compound comprising at least one heteroatom selected from the group consisting of O, S, and N; solv_{b}, when present, is a coordinated solvent molecule that is selected from the group consisting of H₂O; ROH; ROR; RC(O)OR; RC(O)R; RC(S)R; RS(O)R; R₂NC(O)R; and an optionally substituted 5- or 6-membered heterocyclic compound comprising at least one heteroatom selected from the group consisting of O, S, and N; and solv_{c}, when present, represents at least one unbound lattice solvent molecule that is selected from the group consisting of H₂O; ROH; RC(O)R; RC(O)OR; RC(O)R; RC(S)R; RS(O)R; R₂NC(O)R; and an optionally substituted 5- or 6-membered heterocyclic compound comprising at least one heteroatom selected from the group consisting of O, S, and N. When there is more than one solv_{c}, each solv_{c} can be the same or different from another one or more solv_{c}.

Substituent R, at each occurrence, is independently hydrogen or a C₁₋₆-alkyl group. Subscripts x and y, independently of each other, are selected from the integers 0-6 inclusive such that (x+y) is typically 4 or 6, while z is a positive rational number from 0 to 6, inclusive.

Each S-omeprazolato ligand in formula (I), independently of the others, is an anionic ligand of 5-methoxy-2-{[(4-methoxy-3,5-dimethylpyridin-2-yl)methyl]sulfinyl}-1H-benzimidazole or 6-methoxy-2-{[(4-methoxy-3,5-dimethylpyridin-2-yl)methyl]sulfinyl}-1H-benzimidazole (as used herein, the moiety at the 5-position of the former, 5-methoxy compound is referred to as the 5-methoxy group or groups, and the moiety at the 6-position in the 6-methoxy compound is referrd to as the 6-methoxy group or groups, as the case may be).

The invention also provides a magnesium R-omeprazolato coordination complex in the solid state according to formula (II):
[Mg(solvₐ)ₓ(solv_{b})_{y}][Mg(R-omeprazolato)₃]₂.(solv_{c})_{z} (II),
wherein solvₐ, solv_{b}, and solv_{c} are as defined above and the R-omeprazolato ligand in formula (I), independently of the others, is an anionic ligand of 5-methoxy-2-{[(4-methoxy-3,5-dimethylpyridin-2-yl)methyl]sulfinyl}-1H-benzimidazole or 6-methoxy-2-{[(4-methoxy-3,5-dimethylpyridin-2-yl)methyl]sulfinyl}-1H-benzimidazole. Additionally, the invention contemplates magnesium omeprazolato coordination compounds in the solid state that are enantiomerically enriched in either S-omeprazolato or R-omeprazolato ligands. Thus, one embodiment is represented by formula IIIa:
[Mg(solvₐ)ₓ(solv_{b})_{y}][Mg(omeprazolato)₃]₂.(solv_{c})_{z} (IIIa),
wherein there exists an enantiomeric excess of S-omeprazolato ligands over R-omeprazolato ligands. Another embodiment is represented by formula IIIb:
[Mg(solvₐ)ₓ(solv_{b})_{y}][Mg(omeprazolato)₃]₂.(solv_{c})_{z} (IIIb),
wherein there exists an enantiomeric excess of R-omeprazolato ligands over S-omeprazolato ligands.

In yet a further embodiment, the invention provides for a compound of formula IIIc:
[Mg(solvₐ)ₓ(solv_{b})_{y}][Mg(omeprazolato)₃]₂.(solv_{c})_{z} (IIIc),
wherein there exists an equal number of S-omeprazolato and R-omeprazolato ligands.

Another embodiment is a magnesium omeprazolato coordination compound in the solid state according to formula IV:
[Mg(omeprazolato)₃]X (IV),
wherein X is a cation having a +1 charge, and the omeprazolato moiety can be in the S- or R-configuration.

Still another embodiment provides for coordination compounds of formula I wherein at least one of solvₐ, solv_{b}, and solv_{c} represents H₂O. In this embodiment, the values of x, y, and z are selected such that the ratio of total H₂O represented by at least one of solvₐ, solv_{b}, and solv_{c} to Mg ions in formula I ranges from about 1.5 to about 2.4.

Some embodiments of the invention are identified by their association with certain powder X-ray diffraction patterns. Other embodiments are characterized by specific solid-state NMR spectra. These embodiments are described more fully below.

The invention also provides processes for making the metal coordination compounds of formula (I), products that are made by those processes, pharmaceutical compositions comprising the same, and methods of using the same to treat gastric acid related conditions and to inhibit gastric acid secretion.

### BRIEF DESCRIPTION OF THE DRAWINGS

FIG. 1 is an X-ray powder diffractogram of magnesium R-omeprazole as formed by the teachings in Example 6.

FIG. 2 is an X-ray powder diffractogram of magnesium S-omeprazole as formed by the teachings in Example 6.

FIG. 3 is an X-ray powder diffractogram of magnesium R-omeprazole as formed by the teachings in Example 7.

FIG. 4 is an X-ray powder diffractogram of magnesium S-omeprazole as formed by the teachings in Example 8.

FIG. 5A is an ORTEP of Δ,Δ-[Mg(H₂O)₅DMF][Mg(6-methoxy-S-omeprazolato)₃][Mg(6-methoxy-5-omeprazolato)₂(5-methoxy-5-omeprazolato)].DMF (hydrogen atoms not shown for clarity; Magnesium shown with a 30% thermal ellipsoid; all other atoms shown as spheres for clarity).

FIG. 5B is an ORTEP of one Δ-[Mg(6-methoxy-5-omeprazolato)₂(5-methoxy-5-omeprazolato)]⁻ complex with selected atom labels (hydrogen atoms not shown for clarity; Magnesium shown with a 30% thermal ellipsoid; all other atoms shown as spheres for clarity).

FIG. 5c is an ORTEP of one Δ-[Mg(6-methoxy-5-omeprazolato)₃]⁻ complex with selected atom labels (hydrogen atoms not shown for clarity; Magnesium shown with a 30% thermal ellipsoid; all other atoms shown as spheres for clarity).

FIG. 6 is an X-ray powder diffractogram of magnesium S-omeprazole as formed by the teachings in Example 17.

FIG. 7 is an X-ray powder diffractogram of magnesium S-omeprazole as formed by the teachings in Example 18.

FIG. 8 is an X-ray powder diffractogram of magnesium S-omeprazole as formed by the teachings in Example 19.

FIG. 9 is an X-ray powder diffractogram of magnesium S-omeprazole as formed by the teachings in Example 20.

FIG. 10 is an X-ray powder diffractogram of magnesium S-omeprazole as formed by the teachings in Example 22.

FIG. 11 is an X-ray powder diffractogram of magnesium S-omeprazole as formed by the teachings in Example 23.

FIG. 12 is an X-ray powder diffractogram of magnesium S-omeprazole as formed by the teachings in Example 24.

FIG. 13 is an X-ray powder diffractogram of magnesium S-omeprazole as formed by the teachings in Example 25.

FIG. 14 is an X-ray powder diffractogram of magnesium S-omeprazole as formed by the teachings in Example 26 before drying.

FIG. 15 is an X-ray powder diffractogram of magnesium S-omeprazole as formed by the teachings in Example 26 after drying.

FIG. 16 is an X-ray powder diffractogram of magnesium S-omeprazole as formed by the teachings in Example 27.

FIG. 17 is an X-ray powder diffractogram of magnesium S-omeprazole as formed by the teachings in Example 28.

FIG. 18 is an X-ray powder diffractogram of magnesium S-omeprazole as formed by the teachings in Example 29.

FIG. 19 is an X-ray powder diffractogram of magnesium S-omeprazole as formed by the teachings in Example 30.

FIG. 20A is an ORTEP of the mer-[Mg(H₂O)₃(DMSO)₃]-Δ,Δ-[Mg(6-methoxy-5-omeprazolato)₃]₂.(H₂O)₃ (hydrogen atoms are not shown for clarity; Magnesium shown with a 30% thermal ellipsoid; all other atoms shown as spheres for clarity; the resolved disorder in the molecule indicates that predominantly 6-methoxy-S-omeprazolato ligands are present).

FIG. 20B is an ORTEP of one Δ-[Mg(6-methoxy-5-omeprazolato)₃]⁻ complex with selected atom labels (hydrogen atoms not shown for clarity; Magnesium shown with a 30% thermal ellipsoid; all other atoms shown as spheres for clarity).

### DETAILED DESCRIPTION

Definitions

The term “omeprazole”, as used herein unless specified otherwise, refers to a racemic mixture of 5-methoxy-2-{[(4-methoxy-3,5-dimethylpyridin-2-yl)methyl]sulfinyl}-1H-benzimidazole and 6-methoxy-2-{[(4-methoxy-3,5-dimethylpyridin-2-yl)methyl]sulfinyl}-1H-benzimidazole in the solid state. As used herein, “omeprazole” is also represented as 5(6)-methoxy-2-{[(4-methoxy-3,5-dimethylpyridyl-2-yl)methyl]sulfinyl}-1H-benzimidazole. The term “omeprazole,” as specified herein, may additionally refer to 5-methoxy-2-{[(4-methoxy-3,5-dimethylpyridin-2-yl)methyl]sulfinyl}-1H-benzimidazole, 6-methoxy-2-{[(4-methoxy-3,5-dimethylpyridin-2-yl)methyl]sulfinyl}-1H-benzimidazole, or combinations of the 5-methoxy and 6-methoxy isomers, each in amorphous, crystalline, or a combination of crystalline and amorphous forms.

The term “omeprazolate,” as used herein unless specified otherwise, refers to the anion of omeprazole.

The term “S-omeprazole” or “esomeprazole”, as used herein unless specified otherwise, refers to the S stereoisomer of omeprazole.

The term “R-omeprazole”, as used herein unless specified otherwise, refers to the R stereoisomer of omeprazole.

The term “S-omeprazolato”, as used herein unless specified otherwise, refers to the S stereoisomer of the coordinated ligand of S-omeprazole.

The term “R-omeprazolato”, as used herein unless specified otherwise, refers to the R stereoisomer of the coordinated ligand of R-omeprazole.

The terms “S_{P}” and “R_{P}”, as used herein unless specified otherwise, refer to stereoisomers resulting from the arrangement of out-of-plane groups with respect to a plane. Thus, S_{P} refers to a solid state configuration in which bonds to the plane spiral away and down in a clockwise fashion, whereas R_{P} denotes the counterclockwise solid state configuration. In the present context, the person of ordinary skill will appreciate that the pyridyl ring of a S-omeprazolato ligand represents the plane for purposes of determining the configuration.

The term “C₁₋₆-alkyl” refers to a straight or branched alkyl group having from 1 to 6 carbon atoms. Exemplary alkyl groups include but are not limited to methyl, ethyl, n-propyl, iso-propyl, n-butyl, and iso-butyl.

The term “C₆₋₁₂-aryl” refers to an aromatic, optionally fused, carbocyclic moiety having from 6 to 12 carbon atoms. Examples of C₆₋₁₂-aryl include but are not limited to phenyl and naphthyl.

The term “enantiomeric excess,” as used herein, refers generally to the concentration of one stereoisomer that exceeds the concentration of another stereoisomer. Typically, the term is used to characterize the optical purity of an optically active compound that exists in the bulk as two or more stereoisomers. In the present context, the term also refers to the excess of either S- or R-omeprazolato ligands over the other that are present in a given coordination compound of the present invention. Both of these possibilities are contemplated.

The term heterocycle or heterocyclic compound, as used herein, represents a stable 5- to 7-membered monocyclic or stable 8- to 11-membered bicyclic heterocyclic ring which is either saturated or unsaturated, and which consists of carbon atoms and from one to four heteroatoms selected from the group consisting of N, O, and S, and including any bicyclic group in which any of the above-defined heterocyclic rings is fused to a benzene ring. The heterocyclic ring may be attached at any heteroatom or carbon atom which results in the creation of a stable structure. Examples of such heterocyclic compounds include, but are not limited to, azepine, benzimidazole, benzisoxazole, benzofurazan, benzopyran, benzothiopyran, benzofuran, benzothiazole, benzothiene, benzoxazole, benzopyrazole, chromane, cinnoline, dibenzofuran, dihydrobenzofuran, dihydrobenzothiene, dihydrobenzothiopyran, dihydrobenzothiopyran sulfone, furanyl, imidazolidine, imidazoline, imidazole, indoline, indole, isochromane, isoindoline, isoquinoline, isothiazolidine, isothiazole, isothiazolidine, morpholine, naphthyridine, oxadiazole, 2-oxoazepine, 2-oxopiperazine, 2-oxopiperdine, 2-oxopyrrolidine, 2-oxopyridine, 2-oxoquinoline, piperidine, piperazine, pyridine, pyrazine, pyrazolidine, pyrazole, pyridazine, pyrimidine, pyrrolidine, pyrrole, quinazoline, quinoline, quinoxaline, tetrahydrofuran, tetrahydroisoquinoline, tetrahydroquinoline, thiamorpholine, thiamorpholine sulfoxide, thiazole, thiazoline, thienofuran, thienothiene, thiene, and triazole.

Compounds

The inventors discovered that the anion of omeprazole or an optical isomer thereof generally does not combine with magnesium(II) to form a salt as taught in the art, but rather coordinates as omeprazolato ligands to magnesium(II) to form a coordination compound represented by formula (I):
[Mg(solvₐ)ₓ(solv_{b})_{y}][Mg(S-omeprazolato)₃]₂.(solv_{c})_{z} (I),
In accordance with general chemical principles, a compound represented by formula (I) is itself a coordination compound and may itself be considered as a salt of coordination complexes, but the portion of the compound containing S-omeprazolato ligands is a coordination complex. In this context, one of the magnesium(II) center complexes a total of 4 to 6 solvent molecules represented by Solvₐ and solv_{b}, the individual number of complexed solvent molecules being designated by x and y, respectively. A typical sum of x and y is 6, which corresponds to a six-coordinate magnesium(II) species. In maintaining overall charge neutrality, therefore, the compound of formula (I) incorporates two additional magnesium(II) coordination complexes that each bear three S-omeprazolato ligands, giving each such coordination complex a formal charge of −1. Solvents solvₐ, solv_{b}, and solv_{c} need not be the same, and in some cases that are described below they are often not the same.

Compounds of formula (I) also may contain one or more different lattice solvents denoted as solv_{c}. In the context of this invention, solv_{c}, if present, accounts for solvates, that is, those compounds for which the bulk material contains solvent molecules that are not associated with any of the magnesium(II) centers in formula (I). Common examples of such solvated species are crystalline materials in which solvent molecules are trapped in lattice holes within the crystalline structure. Polymorphs or amorphous forms of the compounds may also comprise solvents. Underlying the notion that the number of solv_{c} in formula (I) is not subject to the strictures of bonding principles governing the identity of the magnesium(II) centers is the possibility that solv_{c} can be present in fractional amounts, that is, where z is a positive rational number from 0 to 6, inclusive. Additionally, each solv_{c}, if there is more than one, can be the same or different from the other solvents solvₐ, solv_{b}, or solv_{c}.

Solvents solvₐ, solv_{b}, and solv_{c} are independently selected from the group consisting of H₂O; ROH; ROR; RC(O)OR; RC(O)R; RC(S)R; RS(O)R; and R₂NC(O)R. One or more of the mentioned solvents can also be an optionally substituted 5- or 6-membered heterocyclic compound comprising at least one heteroatom selected from the group consisting of O, S, and N. Substituent R, at each occurrence, is independently hydrogen or a C₁₋₆-alkyl group. The alkyl groups can be straight or branched. Typical alkyl groups, when present, thus include but are not limited to methyl, ethyl, propyl and isopropyl, butyl, sec-butyl, tert-butyl, pentyl, and hexyl. Routinely used alkyl groups are methyl and ethyl.

Exemplary solvents represented by solvₐ, solv_{b}, and solv_{c} can, more specifically, include but are not limited to water, dimethylsulfoxide (“DMSO”), N,N-dimethylformamide (“DMF”), acetone, and C₁₋₆-alkyl alcohols such as methanol and ethanol. Thus in one set of embodiments, solvₐ, solv_{b}, and solv_{c} are independently selected from DMF and water, for example where solvₐ is water while solv_{b} and solv_{c} each are DMF. Alternatively, solvₐ, solv_{b}, and solv_{c} are independently selected from DMSO and water. In yet other embodiments, at least one of solvₐ, solv_{b}, and solv_{c} is water, DMSO, acetone, methanol, or ethanol.

As mentioned above, a typical sum of x and y is 6 for the [Mg(solvₐ)ₓ(solv_{b})_{y}]²⁺ coordination complex, corresponding to an octahedral coordination environment about magnesium(II) for this complex. The skilled artisan will appreciate that depending upon the identities of solvₐ and solv_{b}, together with the individual values for x and y, a given definition of these variables can give rise to various geometric isomers of the complex. For example, complexes of the type [Mg(solvₐ)₄(solv_{b})₂]²⁺ or [Mg(solvₐ)₂(solv_{b})₄]²⁺ can exist as cis and trans isomers. Alternatively, complexes of the type [Mg(solvₐ)₃(solv_{b})₃]²⁺ can give rise to fac and mer isomers, referring to the facial or meridional arrangement, respectively, of the solvₐ and solv_{b} ligands. The invention contemplates all of these possibilities. Additionally, steric bulk from large solvents solvₐ and solv_{b} may result in distortions from an ideal octahedral environment. A representative isomer in this regard is mer-[Mg(solvₐ)₃(solv_{b})₃]²⁺, such as, for example, mer-[Mg(H₂O)₃(DMSO)₃]²⁺.

Compounds according to formula (I) exhibit chirality in four respects. First, the sulfur atom in each S-omeprazolato ligand is a stereogenic center. In this regard, one subset of compounds is one in which at least one, three, five or typically six sulfur atoms are the S stereoisomer. Alternatively, at least one, and in some embodiments all, of the sulfur atoms are the R stereoisomer. Thus, the invention contemplates all combinations of sulfur stereoisomers. Compounds in which all of the sulfur atoms are the R-stereoisomer are represented by formula II:
[Mg(solvₐ)ₓ(solv_{b})_{y}][Mg(R-omeprazolato)₃]₂.(solv_{c})_{z} (II).

It is possible, however, that a compound of the present invention does not contain purely S- or R-omeprazolato ligands, but rather is enriched in one over the other. The resultant mixture of ligands thus gives rise to magnesium (II) omeprazolato coordination complexes that exhibit an enantiomeric excess of either S- or R-omeprazolato ligands. The invention therefore contemplates compounds according to formula IIIa:
[Mg(solvₐ)ₓ(solv_{b})_{y}][Mg(omeprazolato)₃]₂.(solv_{c})_{z} (IIIa),
in which the omeprazolato ligands are enriched in the S stereoisomer, and compounds according to formula IIIb,
[Mg(solvₐ)ₓ(solv_{b})_{y}][Mg(omeprazolato)₃]₂.(solv_{c})_{z} (IIIb),
in which the omeprazolato ligands are enriched in the R stereoisomer.

Also contemplated are compounds of the invention according to formula IIIc:
[Mg(solvₐ)ₓ(solv_{b})_{y}][Mg(omeprazolato)₃]₂.(solv_{c})_{z} (IIIc),
in which there exists an equal number of R-omeprazolato and S-omeprazolato ligands. The skilled person will appreciate that the equality should pertain regardless of the specific stereochemical configurations of omeprazolato ligands that are coordinated to given Mg²⁺ ion, so long as the overall ratio of R- to S-omeprazolato ligands in Formula IIIc remains undisturbed.

A feature that is common to the coordination compounds described herein is a Mg(omeprazolato)₃⁻ complex. Thus, the invention contemplates in another embodiment a neutral form of this compound that is provided as a magnesium omeprazolato coordination compound in the solid state according to formula IV:
[Mg(omeprazolato)₃]X (IV),
Moiety X is charge-balancing, singly charged cation. Suitable cations include but are not limited to Group I cations such as, for example, Li⁺, Na⁺ and K⁺. Also suitable are complex cations such as ammonium (NH₄⁺) and substituted derivatives thereof. It may not always be practical or necessary to identify the spatial arrangements in Formula I between each Mg ion and solvₐ, solv_{b}, and solv_{c}, when present. Thus, for example, it is possible to describe the presence of solvent molecules in a coordination compound of formula I in empirical terms. One embodiment of the invention therefore is a dihydrate, which as defined herein refers to a coordination compound of formula I wherein at least one of solvₐ, solv_{b}, and solv_{c} represents H₂O. Variables x, y, and z are selected such that the total number of water molecules relative to the number of magnesium ions present in formula I ranges from about 1.5 to about 2.4. The skilled person will appreciate that numerous combinations of solvₐ, solv_{b}, and solv_{c}, x, y, and z will satisfy the conditions of the dihydrate as defined above. All of these combinations are contemplated.

The coordination compounds of formula (I) are also chiral in the solid state with respect to the pyridyl group as a whole in each S-omeprazolato ligand. This is so because the 3- and 5-methyl substituents on the pyridyl group constrain the 4-methoxy substituent to lie either above or below the plane of the pyridine ring. Consequently, the pyridyl group introduces a structural chirality when the S-omeprazolato ligand is bound to the magnesium(II) center. The two resultant stereochemical configurations are herein designated as S_{P} and R_{P}. In some embodiments, at least one, at least 3, at least 5, and typically all of the pyridyl rings exist in the S_{P} configuration.

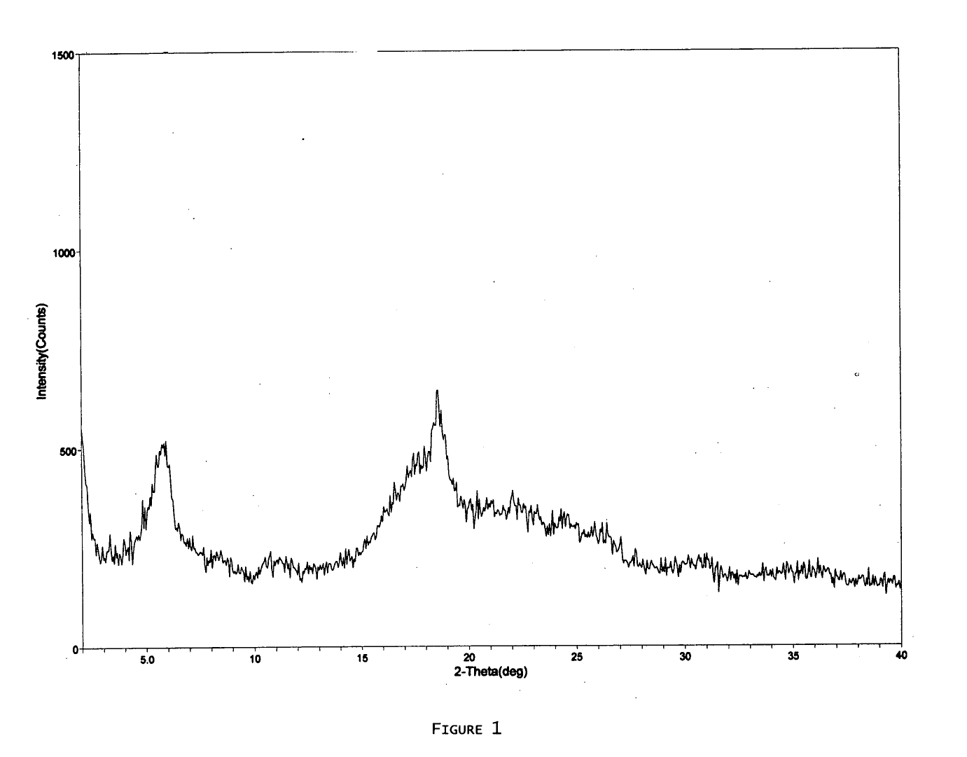

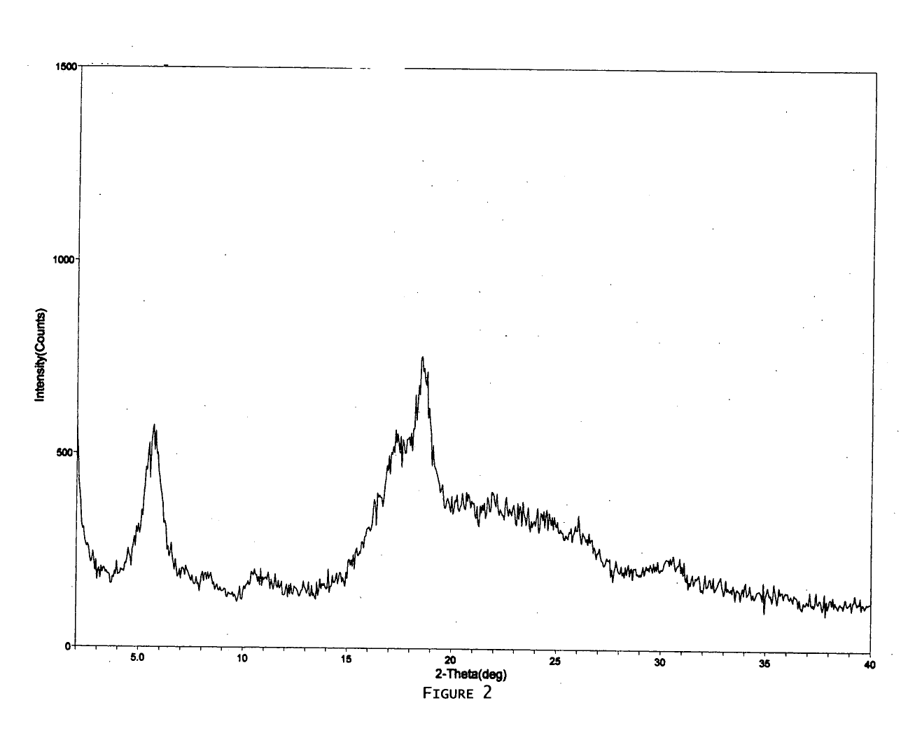

A third aspect in which coordination compounds of formula (I) exhibit chirality arises from the possible optical isomers created by the chiral magnesium(II) coordination polyhedron in each [Mg(S-omeprazolato)₃]⁻ complex. Referring to FIGS. 1B and 2B, for example, each S-omeprazolato ligand behaves as a bidentate ligand as a consequence of it coordinating to magnesium(II) through one benzimidazole nitrogen atom and the oxygen atom in the sulfoxide moiety. The presence of three such ligands in an octahedral coordination environment thus gives rise to two possible propeller shaped optical isomers referred to herein as the Δ and Λ stereoisomers. Consistent with these conventional designations, the Δ stereoisomer thus would appear to screw into a plane, while the Λ stereoisomer would appear to screw out of a plane, when rotated clockwise. In some embodiments, at least one and typically each [Mg(S-omeprazolato)₃]⁻ complex is present as the Δ stereoisomer. Alternatively, at least one and typically each [Mg(S-omeprazolato)₃]⁻ complex is present as the Λ stereoisomer.

A fourth respect in which coordination compounds of formula (I) exhibit chirality arises from the possible optical isomers that result from the bidentate binding nature of the S-omeprazolato ligands. Each S-omeprazolato ligand is a bidentate ligand as a consequence of it coordinating to magnesium(II) through one benzimidazole nitrogen atom and the oxygen atom in the sulfoxide moiety. The presence of such bidentate ligands thus gives rise to two possible orientations, denoted δ and λ, of the atoms in the ligand backbone that are not directly coordinated to magnesium(II). Thus, when the S-omeprazolato ligand is oriented such that the N and O donor atoms and magnesium(II) lie in a plane that is perpendicular to the viewing plane, then the δ chelate ring conformation places the benzimidazole aromatic carbon atom below the aromatic system of the pyridine ring and the S atom above this viewing plane. By contrast, the λ chelate ring conformation places the aromatic carbon of the benzimidazole system above the aromatic pyridine ring and the S atom below the viewing plane.

Coordination compounds according to formula (I) also account for two possible structural isomers of the S-omeprazolato ligand with respect to the methoxy substituent on the benzimidazole moiety. It is known in the art that omeprazole, when in solution, tautomerizes to place the N—H proton on one of the two benzimidazole nitrogen atoms, thereby often yielding a mixture of 5-methoxy-2-{[(4-methoxy-3,5-dimethylpyridin-2-yl)methyl]sulfinyl}-1H-benzimidazole and 6-methoxy-2-{[(4-methoxy-3,5-dimethylpyridin-2-yl)methyl]sulfinyl}-1H-benzimidazole. Solid state properties, and methods for enriching a mixture of the same in one isomer, are described, for example, in U.S. Pat. No. 6,444,689 to Whittle et al. Compounds of formula (I) therefore accommodate S-omeprazolato ligands that bear 5- and 6-methoxy substituents on the benzimidazole moieties. In some embodiments, at least one, at least three, at least four, or at least five S-omeprazolato ligands bear 6-methoxy groups. An exemplary embodiment is where each S-omeprazolato ligand bears a 6-methoxy group. Where applicable, the remaining S-omeprazolato ligands bear 5-methoxy groups. For the four preceeding paragraphs, the invention also contemplates replacing each instance of the term “S-omeprazole” with the term “R-omeprazole” as additional embodiments.

Other embodiments provide for coordination compounds according to formula (I) where all of the sulfur atoms are the S- or R-stereoisomers, at least four or at least five S-omeprazolato ligands bear 6-methoxy groups, and each [Mg(S-omeprazolato)₃]⁻ complex is present as the Λ stereoisomer. Exemplary compounds in this regard include but are not limited to:
Δ,Δ-[Mg(H₂O)₅DMF][Mg(6-methoxy-5-omeprazolato)₃][Mg(6-methoxy-S-omeprazolato)₂(5-methoxy-5-omeprazolato)].DMF;
Δ,Δ-[Mg(H₂O)₅DMF][Mg(6-methoxy-5-omeprazolato)₃][Mg(6-methoxy-S-omeprazolato)₂(5-methoxy-5-omeprazolato)].H₂O;
Δ,Δ-[Mg(H₂O)₅DMF][Mg(6-methoxy-5-omeprazolato)₃][Mg(6-methoxy-S-omeprazolato)₂(5-methoxy-5-omeprazolato)].(H₂O)_{z}(DMF)_{z}; and
mer-[Mg(H₂O)₃(DMSO)₃]-Δ,Δ-[Mg(5(6)-methoxy-5-omeprazolato)₃]₂.(H₂O)₃.
   Processes for Preparing

The coordination compounds represented by formula (I) may be prepared by various methods as described below. In general, the methods entail carrying out synthetic procedures in solution, but result in optically pure solid state products with respect to the chiral sulfur atom in each S-omeprazolato ligand.

One embodiment thus comprises applying to a chromatography column a racemic mixture of 5(6)-methoxy-2-{[(4-methoxy-3,5-dimethylpyridin-2-yl)methyl]sulfinyl}-1H-benzimidazole that is dissolved in a first solvent. The person of skill in the art will recognize that this compound tautomerizes in solution, which furnishes a mixture of the 5- and 6-methoxy isomers as taught, for example, by U.S. Pat. No. 6,444,689 to Whittle et al. The chromatography column can be one of many standard columns that accommodates supercritical fluids such as, for example, supercritical CO₂. As one example, the column is packed with a chiral chromatographic sorbent to facilitate the separation of optical isomers. However, such chromatographic separation is not limited to supercritical fluid but can be accomplished by chiral separation techniques well known to the skilled artisan.

The mixture as described above is then eluted through the column with an eluant comprising either a mobile phase, or a supercritical fluid such as CO₂, and one or more optional co-solvents and/or salts thereof that enhance the solubility, stabilization, separation, or combination thereof for a mixture of compounds. Suitable co-solvents in this regard include but are not limited to C₁₋₆-alkyl alcohols such as, for example, methanol and ethanol. In one embodiment, the eluant comprises a mixture of co-solvents that further include one or more amines. Suitable amines in this in this regard include but are not limited to tertiary amines according to the formula NR¹R²R³ wherein R¹, R², and R³ are independently selected from H and C₁₋₆-alkyl. Examples of amines include but are not limited to dimethylamine, triethylamine, and dimethylethylamine. The eluant may also comprise acid addition salts of the foregoing amines. These include, for example, acetates and halides such as chloride, bromide, and iodide. An example of an acid addition salt is ammonium acetate. In this context, separate fractions of S-omeprazole and R-omeprazole may be collected from the column as mixtures of 5- and 6-methoxy isomers.

S-Omeprazole and R-omeprazole each may be used as obtained from the foregoing separation in the preparation of compounds of formula (I). Thus S- or R-omeprazole is reacted with a magnesium source in a second solvent. The magnesium source provides the requisite Mg(II) ions and facilitates the deprotonation of S- or R-omeprazole ligands. In one embodiment, the magnesium source is a Grignard reagent according to the formula XMgR, wherein X is a halide selected from Cl, Br, and I and R is an organic species selected from C₁₋₆-alkyl and C₆₋₁₂-aryl. Many reagents of this type are suitable for the inventive process and are known to those who are skilled in the art. A typical magnesium source in this context is methyl magnesium bromide.

Another suitable magnesium source are reagents according to the formula MgR₂, wherein R is as defined above. As the skilled artisan knows, MgR₂ exists in equilibrium with MgRX and MgX₂. In this regard, it is possible to generate the MgR₂ reagent by displacing the equilibrium away from MgRX. One convenient method for accomplishing this is by the addition of a reagent that will precipitate MgX₂, thereby driving the equilibrium toward MgR₂. A suitable reagent in this regard is 1,4-dioxane.

In another embodiment, the magnesium source is a magnesium(II) alkoxide compound according to the formula Mg(OR⁴)₂, wherein R⁴ is selected from C₁₋₆-alkyl and C₆₋₁₂-aryl. In one embodiment, R⁴ is a C₁₋₆-alkyl. Suitable magnesium alkoxide compounds in this regard include but are not limited to Mg(OMe)₂ and Mg(OEt)₂.

In yet other embodiments of the process, the magnesium source is an inorganic magnesium salt. In some embodiments, the anion(s) in the salt are capable of being readily displaced by the S- or R-omeprazolato ligands. Exemplary magnesium salts thus include but are not limited to any soluble form of magnesium such as, for example, magnesium halides, e.g., MgCl₂, MgBr₂, MgI₂, and mixed halides thereof; magnesium acetate; magnesium sulfate; magnesium phosphate; magnesium formate; magnesium tartrate, and magnesium carbonate.

An alternative procedure according to this invention entails the complexation of optically pure omeprazolate anions in solution. Thus, a racemic mixture of 5(6)-methoxy-2-{[(4-methoxy-3,5-dimethylpyridyl-2-yl)methyl]sulfinyl}-1H-benzimidazole is reacted with an organic base to furnish a racemic mixture of the corresponding omeprazolate salt. Suitable organic bases in this regard include but are not limited to tetraalkylammonium salts of the formula N(R⁵)₄X wherein R⁵ is a C₁₋₆-alkyl and X is a suitable nucleophilic anion such as, for example, OH⁻, (OR⁵)—, (SR⁵)⁻, (PR⁵₂)⁻, and (NR₅)₂⁻. The mixture is then dissolved in a first solvent, for example to form a concentrated solution, and applied to a chromatography column as described above. The mixture is eluted through the column with either a mobile phase or a supercritical fluid and an optional co-solvent according to the procedure selected and outlined above to yield separate fractions of R- and S-omeprazolate anions in solution. The S- or R-omeprazolate anion then may be combined with a magnesium source in a second solvent to give the corresponding magnesium S- or R-omeprazolato complex. Suitable magnesium sources for use in this embodiment include but are not limited to magnesium halides, e.g., MgCl₂, MgBr₂, MgI₂, and mixed halides thereof; magnesium acetate; magnesium sulfate; magnesium phosphate; magnesium formate; magnesium tartrate; and magnesium carbonate.

Coordination compounds according to the present invention also may be prepared by employing a starting material that is enantiomerically enriched, i.e., where the concentration of the R- or S-stereoisomer in the bulk starting material predominates over the other stereoisomer. In this context, the resulting compound of formula I should have at least one, and up to five, omeprazolato ligands that are coordinated to Mg(II) and that are the same stereoisomer. These compounds can be prepared by adapting any of the teachings herein by substituting enantiomerically enriched omeprazole for enantiomerically pure omeprazole, or can result from the physical mixtures of pure or essentially pure enantiomers.

In the foregoing inventive processes, suitable first and second solvents are judiciously selected according to the requirements of the synthetic step. Thus, the first solvent is selected to dissolve the mixture of omeprazole optical isomers or anions thereof. In this regard, the resultant solution typically is as concentrated as possible. Suitable first solvents therefore include but are not limited to aqueous solvents such as water and ammonia and organic solvents. Exemplary organic solvents typically are ketones, such as acetone and methylethyl ketone; nitrites, such as acetonitrile; nitrogen-based solvents, such as dimethylformamide (DMF) and pyridine; aromatic solvents, such as toluene and benzene; alcohols, such as methanol and ethanol; halogenated solvents, such as chloroform and methylene chloride; and sulfur-containing solvents, such as dimethylsulfoxide. Mixtures of two or more of these solvents also may be employed.

The second solvent generally can be selected from the foregoing list subject to the strictures of the reaction between S- or R-omeprazole and a magnesium source. Thus, for example, protic solvents generally should be avoided when using Grignard reagents.

The magnesium S- or R-omeprazolato complexes resulting from the foregoing processes typically are precipitated by, and in some embodiments crystallized from, one or more solvents represented by solvₐ, solv_{b}, and solv_{c} as described above. Specific techniques for crystallization are well-known in the art and include, for example, evaporation, cooling, vapor diffusion, liquid diffusion, and combinations thereof. Regardless of the crystallization technique, the resulting coordination compound of formula (I) typically contains solvent molecules of the solvent(s) employed for crystallization. Thus, for example, crude magnesium S- or R-omeprazolato compounds may contain water and, when crystallized from a different solvent, may contain molecules of that solvent as solvₐ, solv_{b}, and/or solv_{c}. When the magnesium S- or R-omeprazolato compounds are exposed to multiple solvents, the representation of those solvents as solvₐ, solv_{b}, and solv_{c} in the coordination compounds of formula (I) can vary according to, inter alia, crystallization technique and nature of the solvent(s). Exemplary crystallization procedures and resultant compounds are given in the examples below.

As a consequence of the foregoing considerations, compounds of the present invention may exist as clathrates with respect to crystallographic packing to accommodate solvₐ, solv_{b}, and solv_{c}. In accordance with accepted terminology in the art, a clathrate generally relates to inclusion complexes in which molecules of one substance are completely enclosed within the crystal lattice structure of another, and may be interchanged without significantly altering the lattice parameters. Thus in the present context, one or more of solv_{c} may be viewed as being enclosed within the crystal structure of a compound of formula (I). More particularly, as mentioned above, it should be recognized that multiple clathrates may each give rise to substantially the same X-ray powder diffraction pattern, notwithstanding the presence of different solvₐ, solv_{b}, and solv_{c} within each clathrate. Additionally, in such clathrates, solvₐ and/or Solv_{b} can be exchanged with other acceptable solvents of similar shape or size such that the l;attice parameters are maintained in the resulting coordination compound. In this regard, therefore, formula (I) of the present invention accounts for the potential existence of one or more clathrates. Thus, X-ray powder diffraction may not be sufficient to completely determine the composition or fingerprint of such a clathrate.

Pharmaceutical Composition

The invention also contemplates pharmaceutical compositions that comprise a therapeutically effective amount of at least one coordination compound of formula (I) according to this invention and a pharmaceutically acceptable carrier, diluent, excipient, stimulant, or combination thereof, the selection of which is known to the skilled artisan. In one embodiment, a solid pharmaceutical composition of the present invention is blended with at least one pharmaceutically acceptable excipient, diluted by an excipient or enclosed within such a carrier that can be in the form of a capsule, sachet, tablet, buccal, lozenge, paper, or other container. When the excipient serves as a diluent, it may be a solid, semi-solid, or liquid material which acts as a vehicle, carrier, or medium for the compound. Thus, the formulations can be in the form of tablets, pills, powders, elixirs, suspensions, emulsions, solutions, syrups, capsules (such as, for example, soft and hard gelatin capsules), suppositories, lozenges, buccal dosage forms, sterile injectable solutions, and sterile packaged powders.

Examples of suitable excipients include, but are not limited to, starches, gum arabic, calcium silicate, microcrystalline cellulose, polyvinylpyrrolidone, cellulose, water, syrup, and methyl cellulose. The compositions can additionally include lubricating agents such as, for example, talc, magnesium stearate and mineral oil; wetting agents; emulsifying and suspending agents; preserving agents such as methyl- and propyl hydroxybenzoates; sweetening agents; or flavoring agents. Polyols, buffers, and inert fillers may also be used. Examples of polyols include, but are not limited to: mannitol, sorbitol, xylitol, sucrose, maltose, glucose, lactose, dextrose, and the like. Suitable buffers encompass, but are not limited to, phosphate, citrate, tartrate, succinate, and the like. Other inert fillers which may be used encompass those which are known in the art and are useful in the manufacture of various dosage forms. If desired, the solid pharmaceutical compositions may include other components such as bulking agents and/or granulating agents, and the like. The compositions of the invention can be formulated so as to provide normal, sustained, or delayed release of the compound after administration to the patient by employing procedures well known in the art.

In the event that a foregoing composition is to be used for parenteral administration, such a composition typically comprises sterile aqueous and non-aqueous injection solutions comprising the coordination compound of formula I, for which preparations are typically isotonic with the blood of the intended recipient. These preparations may contain anti-oxidants, buffers, bacteriostats, and solutes which render the formulation isotonic with the blood of the intended recipient. Aqueous and non-aqueous sterile suspensions may include suspending agents and thickening agents.

The compositions may be presented in unit-dose or multi-dose containers, for example sealed ampules and vials. Extemporaneous injection solutions and suspensions may be prepared from sterile powders, granules and tablets of the kind previously described.

In some embodiments of the invention, the composition may be made into the form of dosage units for oral administration. The coordination compound of formula (I) may be mixed with a solid, pulverant carrier such as, for example, lactose, saccharose, sorbitol, mannitol, starch, amylopectin, cellulose derivatives or gelatin, as well as with an antifriction agent such as, for example, magnesium stearate, calcium stearate, and polyethylene glycol waxes. The mixture is then pressed into tablets. If coated tablets are desired, the above prepared core may be coated with a concentrated solution of sugar, which may contain gum arabic, gelatin, talc, titanium dioxide, or with a lacquer dissolved in volatile organic solvent or mixture of solvents. To this coating, various dyes may be added in order to distinguish among tablets with different active compounds or with different amounts of the active compound present.

Soft capsules also may be prepared in which capsules contain a mixture of the compound and vegetable oil or non-aqueous, water miscible materials such as, for example, polyethylene glycol and the like. Hard capsules may contain granules of the compound in combination with a solid, pulverulent carrier, such as, for example, lactose, saccharose, sorbitol, mannitol, potato starch, corn starch, amylopectin, cellulose derivatives, or gelatin.

Dosage units for rectal administration may be prepared in the form of suppositories which may contain the coordination compound of formula I in a mixture with a neutral fat base, or they may be prepared in the form of gelatin-rectal capsules which contain the active substance in a mixture with a vegetable oil or paraffin oil.

Liquid preparations for oral administration may be prepared in the form of syrups or suspensions, e.g., solutions containing the coordination compound of formula I, sugar, and a mixture of ethanol, water, glycerol, and propylene glycol. If desired, such liquid preparations may contain coloring agents, flavoring agents, and saccharin. Thickening agents such as carboxymethylcellulose may also be used.

Tablets for oral use are typically prepared in the following manner, although other techniques may be employed. The solid substances are gently ground or sieved to a desired particle size, and the binding agent is homogenized and suspended in a suitable solvent. The coordination compound of formula (I) and auxiliary agents are mixed with the binding agent solution. The resulting mixture is moistened to form a uniform suspension. The moistening typically causes the particles to aggregate slightly, and the resulting mass is gently pressed through a stainless steel sieve having a desired size. The layers of the mixture are then dried in controlled drying units for determined length of time to achieve a desired particle size and consistency. The granules of the dried mixture are gently sieved to remove any powder. To this mixture, disintegrating, anti-friction, and anti-adhesive agents are added. Finally, the mixture is pressed into tablets using a machine with the appropriate punches and dies to obtain the desired tablet size. The operating parameters of the machine may be selected by the skilled artisan.

Typically, preparation of lozenge and buccal dosage forms are prepared by methods known to one of ordinary skill in the art.

In other embodiments, the coordination compound may be present in a core surrounded by one or more layers including, for example, an enteric coating layer with or without a protective sub-coating as known to the ordinarily skilled artisan relative to pharmaceutical formulations. If no sub-coating is employed, then the enteric coating should be selected such that it does not degrade the active ingredient in the core. However, pharmaceutical compositions having a core containing active ingredient and an alkaline reacting compound, a protective sub-coat and an enteric coat are specifically excluded from this embodiment.

The final dosage form encompassing the above embodiments may be either an enteric coated tablet or capsule or in the case of enteric coated pellets, pellets dispensed in hard capsules or sachets or pellets formulated into tablets. It is desirable for long term stability during storage that the water content of the final dosage form containing the coordination compound of formula (I) (enteric coated tablets, capsules or pellets) be minimized. As a consequence, the final package containing hard capsules filled with enteric coated pellets typically also contain a desiccant, which reduces the water content of the capsule shell to a level where the water content of the enteric coated pellets filled in the capsules does not exceed a certain level.

According to some embodiments, the coordination compound and composition of the present invention are formulated in a unit dosage form, each dosage containing from about 10 mg to about 400 mg. The term “unit dosage form” refers to physically discrete units, such as capsules or tablets suitable as unitary dosages for human patients and other mammals, each unit containing a predetermined quantity of one or more coordination compound(s) calculated to produce the desired therapeutic effect, in association with at least one pharmaceutically acceptable carrier, diluent, excipient, or combination thereof. Generally, dosages of the compound in such unit dosage forms are from about 10 mg to about 15 mg, about 20 mg to about 25 mg, about 40 mg to about 80 mg, and about 80 mg to about 400 mg.

Methods of Treatment

The invention also provides methods of treating gastric acid related conditions and gastric acid secretion in a subject suffering from the conditions or secretion comprising administering to the subject a therapeutically effective amount of the coordination compound of formula (I). Alternatively, the method comprises administering the pharmaceutical composition thereof as described above.

In some embodiments, the subject suffering from the condition is an animal, such as, for example, a mammal. Thus in one embodiment the mammal is a human being. Other examples of mammals include but are not limited to monkeys, sheep, bovines, horses, dogs, cats, rabbits, rats, and mice.

As used herein, the term “treatment” or “treating” contemplates partial or complete inhibition of the stated condition or disease state when a coordination compound of formula (I) or its pharmaceutical composition is administered prophylactically or following the onset of the condition for which the compound or composition is administered. For the purposes of this invention, the term “prophylaxis” refers to the administration of the compound to subject to protect the subject from any of the conditions set forth herein.

More specifically, the gastric acid related condition typically is a digestive ulcer (e.g., gastric ulcer, duodenal ulcer, stomal ulcer, Zollinger-Ellison syndrome, etc.), gastritis, reflux esophagitis, NUD (non-ulcer dyspepsia), gastric cancer and gastric MALT lymphoma; Helicobacter pylori eradication. Other conditions include but are not limited to duodenal cancer, heartburn, erosive esophagitis, pathological hypersecretary conditions, duodenitis, non-ulcer dyspepsia, and acute upper gastrointestinal bleeding. The inventive method is also useful for the suppression of upper gastrointestinal hemorrhage due to digestive ulcer, acute stress ulcer and hemorrhagic gastritis; suppression of upper gastrointestinal hemorrhage due to invasive stress (stress from major surgery necessitating intensive management after surgery, and from cerebral vascular disorder, head trauma, multiple organ failure and extensive burns necessitating intensive treatment); treatment and prevention of ulcer caused by a nonsteroidal anti-inflammatory agent (NSAID) or aspirin; treatment and prevention of hyperacidity and ulcer due to postoperative stress; and pre-anesthetic administration.

In particular, the present invention is useful in healing of erosive esophagitis. In patients with gastro-esophageal reflux disease (GERD), stomach acid backs up into the esophagus due to inappropriate relaxation of the lower esophageal sphincter (LES). If GERD is left untreated, the acid can erode the lining of the esophagus since, unlike the stomach, there is no protective lining to protect the esophagus from stomach acid. Once the esophagus is healed from such erosions, the present invention can be used for maintenance of the healed esophagus.

Coordination compounds of formula (I) of the present invention can also be used to treat symptomatic gastroesophageal reflux disease, otherwise known as acid reflux disease. Acid reflux disease occurs when the reflux of stomach acid into the esophagus is frequent enough to impact daily life and/or damage the esophagus. Acid reflux occurs when the lower esophageal sphincter (LES), which normally opens and closes allowing food to enter and prevents the acid in the stomach from backing up into the esophagus, opens at inappropriate times, allowing acid from the stomach to enter the esophagus.

Coordination compounds of formula (I) can also be used to treat duodenal ulcer disease as mentioned above. A duodenal ulcer is a type of peptic disease that is caused by an imbalance between acid and pepsin (an enzyme) secretion and the defenses of the mucosal lining. The inflammation may be precipitated by aspirin and selective or non-selective COX-2 specific inhibitors.

Duodenal ulcers are commonly associated with the presence of the bacteria Helicobacter pylori in the stomach. Risk factors are aspirin and NSAID use, cigarette smoking, and older age. Duodenal ulcer has historically occurred more frequently in men, but more recent data suggest similar rates in both men and women. The lifetime prevalence of a peptic ulcer is 5 to 10% and approaches 10 to 20% in patients who are Helicobacter pylori positive.

The present invention also contemplates a method of inhibiting gastric acid secretion in a subject comprising administering to the subject a therapeutically effective amount of a coordination compound of formula (I) or pharmaceutical composition thereof. While not being bound to any one theory, the inventors believe that the present invention is effective in treating the gastric disorders by acting as a proton pump inhibitor. Proton pump inhibitors suppress gastric acid secretion by specific inhibition of the H⁺/K⁺-ATPase in the gastric parietal cell. By acting on the proton pump, coordination compounds of formula (I) block the last step in acid production which has the overall effect of reducing gastric secretions.

The following examples are proffered merely to illustrate the invention described above; they are not intended to limit in any way the scope of this invention. Throughout the specification, any and all cited publicly available documents are specifically incorporated into this patent application by reference as if fully set forth herein.

### EXAMPLE 1

Preparation of Magnesium S-Omeprazole from S-Omeprazole and Methyl Magnesium Bromide Via Grignard Reaction.

Methyl magnesium bromide (2.1 mL, 6.3 mmol, 3.0 M in diethyl ether) was added by syringe to a Schlenk flask (100 mL) under a nitrogen purge. 1,4-Dioxane (5 mL) was added to the flask in order to precipitate all magnesium salts, leaving dimethyl magnesium in solution. In a separate Schlenk flask (25 mL), S-omeprazole obtained from chiral high performance liquid chromatography (HPLC; 56.60 mg, 0.16 mmol; see Example 15 for conditions) was dissolved in toluene (10 mL). The dimethyl magnesium solution was removed from the Schlenk flask by syringe and gradually added to the S-omeprazole solution. The reaction solution appeared inactive; therefore, an aliquot of methyl magnesium bromide (1.8 mL, 5.4 mmol, 3.0 M in diethyl ether) was added to the flask by syringe and the resulting suspension stirred. A sufficient amount of ice cold water was added to the reaction mixture and the contents of the Schlenk flask transferred to a separatory funnel with a small portion of diethyl ether. The aqueous layer was separated from the organic layer and after washing with water, the organic layer was set aside. The aqueous layers were combined and allowed to stand for 12 hours in an attempt to form crystals. The aqueous sample was then heated to 38° C. for 3 hours. Crystallization was unsuccessful. After removal from heat, the aqueous sample was set aside for approximately 10 days after which time the water was removed by rotary evaporation to form a dense yellow oil. The oil was dissolved in a sufficient amount of dimethylformamide and a small amount of ethyl acetate was added until a white precipitate began to form. The aqueous solution was set aside to allow for further crystallization for 2.5 days.

### EXAMPLE 2

Preparation of Magnesium S-Omeprazole from S-Omeprazole and Methyl Magnesium Bromide Via a Grignard Reaction.

S-Omeprazole (103.06 mg, 0.30 mmol) was separated from rac-omeprazole free base by means of chiral HPLC (see Example 15) and dissolved in sufficient deoxygenated tetrahydrofuran in a clean, dry Schlenk flask (25 mL). Methyl magnesium bromide (2.0 mL, 6.0 mmol, 3.0 Min diethyl ether) was added slowly by syringe. Immediately, the evolution of a gas was observed and the reaction was allowed to stir under ambient conditions for two hours. A small quantity of ice cold water was added to the flask resulting in a vigorous exothermic reaction. Additional water was added and a yellow solid formed. The contents of the flask were transferred to a 1 L separatory funnel with water, diethyl ether, and tetrahydrofuran. An emulsion formed and concentrated ammonium hydroxide was added to the separatory funnel in an amount sufficient to dissipate the emulsion.

### EXAMPLE 3

Preparation of Magnesium S-Omeprazole from S-Omeprazole and Magnesium Methoxide.

Magnesium metal (14.429 mg, 0.5937 mmol) was placed in a small, dry Schlenk flask with methanol (5 mL). The flask was fitted with a nitrogen purge and the solution warmed to 40° C. to dissolve the metal. S-Omeprazole, separated from rac-omeprazole free base by chiral HPLC (see Example 16; 0.09954 g, 0.2882 mmol), was dissolved in methanol (7 mL) and added to the Schlenk flask. The solution was stirred under nitrogen for 48 hours. Water (8 μL) was added to the Schlenk flask and stirred for 30 minutes to facilitate the precipitation of magnesium salts. The magnesium salts were removed by filtration through a Whatman #4 paper filter. Any remaining solids were removed from the pink supernatant solution by filtration through 0.45-μm polytetrafluoroethylene (PTFE). The solution was concentrated by rotary evaporation. Acetone (10 mL) was added and the solution placed under refrigeration for 2 days.

### EXAMPLE 4

Preparation of Magnesium S-Omeprazole and Magnesium R-Omeprazole from S-Omeprazole and R-Omeprazole and Methyl Magnesium Bromide Via Grignard Reaction.

1,4-Dioxane (5 mL) was placed in a three-neck round bottom flask (250 mL) and the solvent deoxygenated with nitrogen. Methyl magnesium bromide (10 mL, 30 mmol, 3.0 Min diethyl ether) was added by syringe to the flask. A white precipitate formed and the resulting mixture of dimethyl magnesium was stirred under nitrogen. Deoxygenated tetrahydrofuran (5 mL) was added to the flask and stirred. R-Omeprazole (500 mg, 1.448 mmol) separated from rac-omeprazole free base by means of SFC (see Example 10), was dissolved in a sufficient amount of tetrahydrofuran and transferred into a Schlenk flask (100 mL). S-Omeprazole (500 mg, 1.448 mol), which was also separated by means of SFC (see Example 10), was dissolved in a sufficient amount of tetrahydrofuran, and placed in another Schlenk flask. At ambient temperature, portions of dimethyl magnesium (approximately 2-3 mL) were added dropwise to the omeprazole solutions by syringe and evolution of a gas was observed. Additional drops of dimethyl magnesium were added to both flasks until the reactions were complete. Any particulate matter was removed by filtration through Whatman #4 paper filters and the supernatant tetrahydrofuran was removed by rotary evaporation producing a solid from each reaction. Each product was dissolved in methanol (10 mL) and placed in a nitrogen cabinet to attempt recrystallization. After approximately 12 hours, the sample solutions were a dark purple color. Attempts to purify the products on silica gel were unsuccessful.

### EXAMPLE 5

Preparation of Magnesium S-Omeprazole and Magnesium R-Omeprazole from S-Omeprazole and R-Omeprazole and Methyl Magnesium Bromide by Grignard Reaction at Low Temperature.

Two clean, dry Schlenk flasks (100 mL) were immersed in a liquid nitrogen/acetone slurry. R-Omeprazole (500 mg, 1.448 mmol) separated from rac-omeprazole by means of SFC (see Example 11), was dissolved in a sufficient amount of deoxygenated toluene and transferred into one of the flasks. S-Omeprazole (500 mg, 1.448 mol), also separated by means of SFC (see Example 11) was dissolved in a sufficient amount of deoxygenated toluene and transferred into the other flask. A pressure equalizing dropping funnel containing toluene was inserted into each Schlenk flask. Methylmagnesium bromide (400 μL, 1.2 mmol, 3.0 M in diethyl ether) was added to each addition funnel via syringe. The methylmagnesium bromide solution was added dropwise into the contents of the Schlenk flasks kept at low temperature. Both solutions were stirred at low temperature for 20 minutes. The magnesium R-omeprazole solution was allowed to warm to room temperature and transferred carefully to a separatory funnel containing cold water. Attempts to dissipate the resulting emulsion using magnesium carbonate and aqueous ammonia were unsuccessful. The toluene fraction was separated, placed in a small round bottom flask and the toluene removed by rotary evaporation. The round bottom flask containing a white solid was placed in a nitrogen cabinet for two days. The cold contents of the magnesium S-omeprazole flask were added to a separatory funnel containing aqueous ammonia (80 mL, 15:1 water: concentrated ammonium hydroxide). The aqueous layer was separated and back extracted with toluene. The organic layer was placed in a 150 mL round bottom flask and set in a nitrogen cabinet. After two days the toluene was decanted from the white solid product. The resulting white solid from each reaction was characterized by means of X-ray powder diffraction (XRD). Based on these data, each product appeared mostly amorphous with a small degree of crystalline character.

### EXAMPLE 6

Preparation of Magnesium S-Omeprazole and Magnesium R-Omeprazole from S-Omeprazole and R-Omeprazole and Magnesium Methoxide.

Magnesium methoxide (3 mL, 2.2 mmol, 7.8 wt % in methanol) was placed in two separate flasks containing R-omeprazole (500 mg, 1.448 mmol) and S-omeprazole (500 mg, 1.448 mmol), which were previously separated by means of SFC (see example 12). An additional portion of methanol (5 mL) was added to each flask and the flasks were placed in an ice bath and stirred for thirty minutes. The flasks were removed from the ice bath and allowed to stir under ambient conditions for approximately 18 hours. A small portion of water (0.02 mL) was added to each flask and the solutions stirred for an additional 30 minutes. Any solids were removed by filtration through 0.45 μm PTFE and the solvents removed by rotary evaporation. Acetone (18 mL) was added to each flask and the solutions stirred for 30 minutes after which the acetone was removed by rotary evaporation. The resulting solid products were characterized by means of X-ray powder diffraction with the results given in Tables 1 and 2. The diffractograms are given in FIG. 1 (magnesium R-omeprazole) and FIG. 2 (magnesium S-omeprazole). Relative peak intensity definitions are given below and are intended to apply to all references to powder X-ray diffraction data here and throughout this description.

| % Relative | |
|---|---|
| Intensity | Definition |
| 25-100 | vs (very strong) |
| 10-25 | s (strong) |
| 3-10 | m (medium) |
| 1-3 | w (weak) |
| <1 | vw (very weak) |

TABLE 1
Positions and intensities of the major peaks in the X-ray powder
diffraction of magnesium R-omeprazole as formed by the teachings
in Example 6.

| d-value/Å | Relative Intensity |
|---|---|
| 15.3 | vs |
| 10.5 | s |
| 8.2 | s |
| 5.0 | s |
| 4.8 | vs |
| 4.0 | s |
| 3.7 | s |
| 2.9 | s |

TABLE 2
Positions and intensities of the major peaks in the X-ray powder
diffraction of magnesium S-omeprazole as formed by the teachings
in Example 6.

| d-value/Å | Relative Intensity |
|---|---|
| 15.5 | vs |
| 10.6 | m |
| 8.4 | s |
| 5.1 | vs |
| 4.8 | vs |
| 3.4 | s |
| 2.9 | s |

### EXAMPLE 7

Preparation of Magnesium R-Omeprazole from R-Omeprazole and Magnesium Ethoxide.

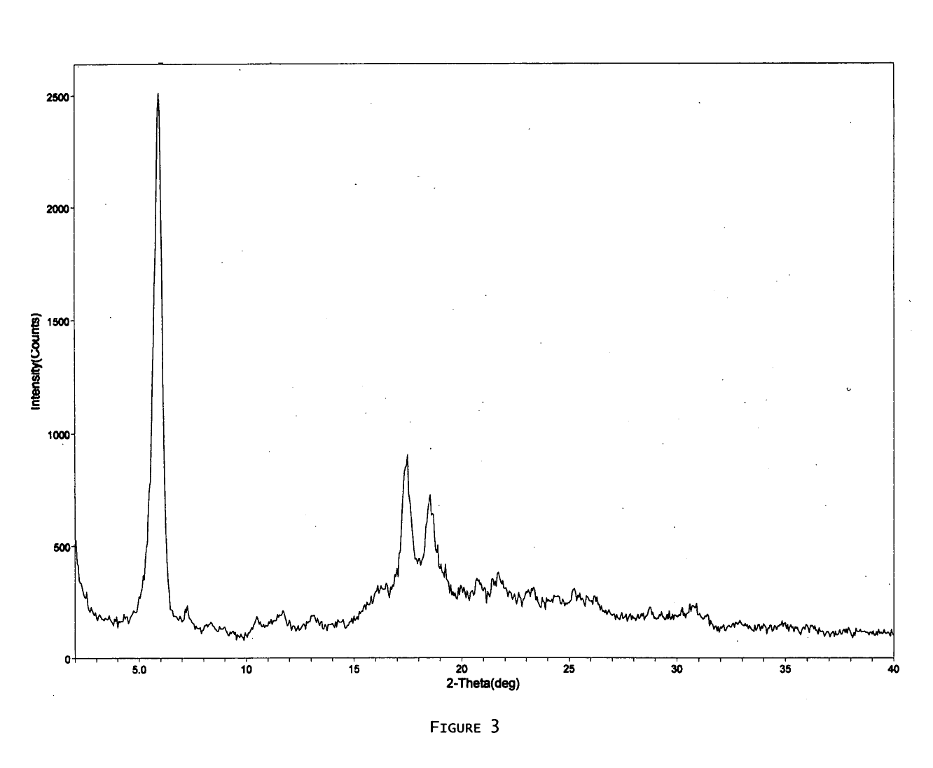

Magnesium ethoxide (85.003 mg, 0.7428 mmol) was combined with R-omeprazole (500 mg in 10 mL ethanol, 1.448 mmol) obtained from the chiral separation of rac-omeprazole (SFC; see Example 13) and methanol (50 mL). The solution was allowed to stir for approximately 48 hours. A small portion of water was added (0.5 mL) and the solution was allowed to stir for an additional hour. Any particulate matter was removed by filtration through 0.45 μm PTFE and the solvent removed by rotary evaporation. The flask was sealed and refrigerated for approximately 18 hours. Acetone (18 mL) was added and the solution allowed to stir for approximately two hours. The acetone was then removed by rotary evaporation. The resulting solid product was characterized by means of X-ray powder diffraction with the results given in Table 3. The diffractogram is given in FIG. 3.

TABLE 3
Positions and intensities of the major peaks in the X-ray powder
diffraction of magnesium R-omeprazole as taught in Example 7.

| d-value/Å | Relative Intensity |
|---|---|
| 14.8 | vs |
| 12.2 | w |
| 10.8 | w |
| 8.4 | w |
| 7.6 | m |
| 6.7 | w |
| 5.5 | w |
| 5.1 | s |
| 4.8 | s |
| 4.3 | m |
| 4.1 | m |
| 3.8 | w |
| 3.5 | w |
| 2.9 | m |

### EXAMPLE 8

Preparation of Magnesium S-Omeprazole from S-Omeprazole and Magnesium Ethoxide.

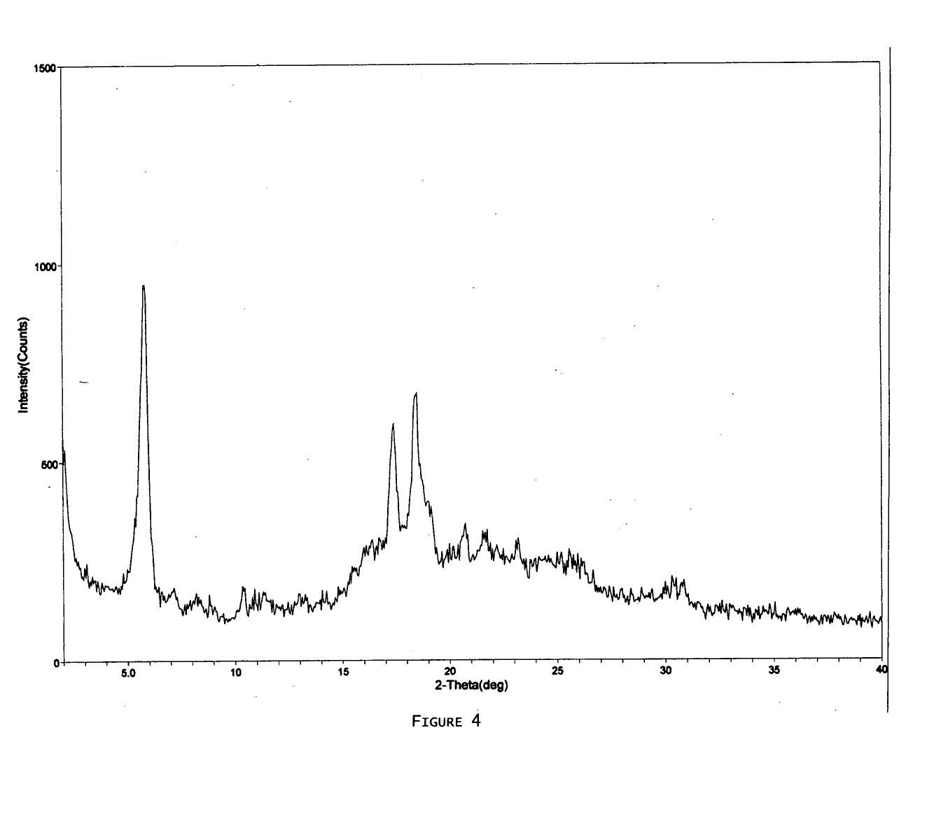

Magnesium ethoxide (100.61 mg, 0.8792 mmol in 20 mL methanol) was combined with S-omeprazole (500 mg in 10 mL ethanol, 1.448 mmol) obtained from the chiral separation of rac-omeprazole (SFC; see Example 13). The solution was allowed to stir for approximately 18 hours. A small portion of water was added (0.1 mL) and the solution was allowed to stir for an additional two hours. Any particulate matter was removed by filtration through 0.45 μm PTFE and the solvent removed by rotary evaporation. Acetone (18 mL) was added and the solution allowed to stir for approximately one hour. The acetone was removed by rotary evaporation. The resulting solid product was characterized by means of X-ray powder diffraction. The diffractogram is given in FIG. 4.

TABLE 4

Positions and intensities of the major peaks in the X-ray powder
diffraction of magnesium S-omeprazole as taught in Example 8.

| d-value/Å | Relative Intensity |
|---|---|
| 15.1 | vs |
| 12.5 | m |
| 10.8 | m |
| 10.0 | m |
| 8.5 | m |
| 7.8 | m |
| 5.1 | vs |
| 4.8 | vs |
| 4.3 | m |
| 4.1 | m |
| 3.8 | m |
| 3.4 | m |
| 2.9 | m |

### EXAMPLE 9

Preparation of Magnesium S-Omeprazole and Magnesium R-Omeprazole from S-Omeprazole and R-Omeprazole and Methyl Magnesium Bromide by Grignard Reaction.

Two clean, dry Schlenk flasks (100 mL) were immersed in a liquid nitrogen/acetone slurry. R-Omeprazole (500 mg; 1.448 mmol) and S-omeprazole (500 mg, 1.448 mmol) previously separated by means of SFC (Example 14) were placed in their respective flasks with an appropriate amount of toluene. Each flask was fitted with a dropping funnel containing deoxygenated toluene (10 mL) and methylmagnesium bromide (400 μL, 1.2 mmol, 3.0 M in diethyl ether). The solution was added dropwise to the omeprazole solutions and the flasks held at low temperature for an additional 30 minutes after complete addition of the Grignard solution. Both flasks were allowed to warm to room temperature and the contents of each flask transferred to individual separatory funnels containing an appropriate amount of water. The organic layer was removed and placed in a round bottom flask (200 mL). The aqueous layer was backwashed with toluene, separated and the organic layers combined into a round bottom flask. The solvent from the magnesium S-omeprazole flask was reduced by rotary evaporation (10 mL), the flask placed under refrigerated conditions for approximately 18 hours, then placed under a nitrogen purge to remove the solvent. The product was a dark purple oil. The magnesium R-omeprazole flask was placed directly under refrigerated conditions without removal of solvent for approximately 18 hours. The solvent was then removed by rotary evaporation resulting in a dark purple oil.

### EXAMPLE 10

Preparation of R-Omeprazole and S-Omeprazole from rac-Omeprazole by Means of Chiral Supercritical Fluid Chromatography.

Omeprazole (2517.6 mg; 7.289 mmol) was placed in a volumetric flask (100 mL) with 0.4% triethylamine (TEA) in methanol, dissolved by means of sonication, and brought to volume. The solution was injected onto a Berger Multigram Supercritical Fluid System under the following conditions:
Column: Chiralpak AS-H SFC
Column Dimensions: 20 mm×250 mm; 5 μm particle size
Column Temperature: 35° C.
Column Pressure: 100 bar
Detection: 302 nm
Flow rate: 50 mL/minute
Mobile Phase: 75:25 Carbon Dioxide: Methanol with 0.4% TEA
Injection Volume: 0.75 mL

The fractions of each enantiomer were collected into separate ice chilled flasks. After collection, the solvent was removed by rotary evaporation and the resulting oils were used directly in a subsequent experiment (see Example 4).

### EXAMPLE 11

Preparation of R-Omeprazole and S-Omeprazole from rac-Omeprazole by Means of Chiral Supercritical Fluid Chromatography.

Omeprazole (2589.3 mg; 7.496 mmol) was placed in a volumetric flask (100 mL) with 0.4% triethylamine (TEA) in methanol, dissolved by means of sonication, and brought to volume. The solution was injected onto a Berger Multigram Supercritical Fluid System under the following conditions:
Column: Chiralpak AS-H SFC
Column Dimensions: 20 mm×250 mm; 5 μm particle size
Column Temperature: 35° C.
Column Pressure: 100 bar
Detection: 302 nm
Flow rate: 50 mL/minute
Mobile Phase: 75:25 Carbon Dioxide: Methanol with 0.4% TEA
Injection Volume: 0.75 mL

The fractions of each enantiomer were collected into separate ice chilled flasks. After collection, the solvent was removed by rotary evaporation and the resulting oils were used directly in a subsequent experiment (see Example 5).

### EXAMPLE 12

Preparation of R-Omeprazole and S-Omeprazole from rac-Omeprazole by Means of Chiral Supercritical Fluid Chromatography.

Omeprazole (2552.4 mg; 7.389 mmol) was placed in a volumetric flask (100 mL) with 0.3% dimethylethylamine (DMEA) in methanol, dissolved by means of sonication, and brought to volume. The solution was injected onto a Berger Multigram Supercritical Fluid System under the following conditions:
Column: Chiralpak AS-H SFC
Column Dimensions: 20 mm×250 mm; 5 μm particle size
Column Temperature: 35° C.
Column Pressure: 100 bar
Detection: 302 nm
Flow rate: 50 mL/minute
Mobile Phase: 75:25 Carbon Dioxide: Methanol with 0.3% DMEA
Injection Volume: 0.75 mL

The fractions of each enantiomer were collected into separate ice chilled flasks. After collection, the solvent was removed by rotary evaporation. The resulting oils were used in a subsequent experiment (see Example 6).

### EXAMPLE 13

Preparation of R-Omeprazole and S-Omeprazole from rac-Omeprazole by Means of Chiral Supercritical Fluid Chromatography.

Omeprazole (2463.0 mg; 7.130 mmol) was placed in a volumetric flask (200 mL) with 0.3% dimethylethylamine (DMEA) in ethanol, dissolved by means of sonication, and brought to volume. The solution was injected onto a Berger Multigram Supercritical Fluid System under the following conditions:
Column: Chiralpak AS-H SFC
Column Dimensions: 20 mm×250 mm; 5 μm particle size
Column Temperature: 35° C.
Column Pressure: 150 bar
Detection: 302 nm
Flow rate: 50 mL/minute
Mobile Phase: 75:25 Carbon Dioxide: Ethanol with 0.3% DMEA
Injection Volume: 1.0 mL

The fractions of each enantiomer were collected into separate ice chilled flasks. After collection, the solvent was reduced by rotary evaporation to approximately 10 mL and the diluted products used in a subsequent experiment (see Examples 7 and 8).

### EXAMPLE 14

Preparation of R-Omeprazole and S-Omeprazole from rac-Omeprazole by Means of Chiral Supercritical Fluid Chromatography.

Omeprazole (2556.3 mg; 7.401 mmol) was placed in a volumetric flask (100 mL) with 0.3% dimethylethylamine (DMEA) in methanol, dissolved by means of sonication, and brought to volume. The solution was injected onto a Berger Multigram Supercritical Fluid System under the following conditions:
Column: Chiralpak AS-H SFC
Column Dimensions: 20 mm×250 mm; 5 μm particle size
Column Temperature: 35° C.
Column Pressure: 100 bar
Detection: 302 nm
Flow rate: 50 mL/minute
Mobile Phase: 75:25 Carbon Dioxide: Methanol with 0.3% DMEA
Injection Volume: 0.75 mL

The fractions of each enantiomer were collected into separate ice chilled flasks. After collection, the solvent was removed by rotary evaporation and the resulting oils used directly in a subsequent experiment (see Example 9).

### EXAMPLE 15

Preparation of S-Omeprazole from rac-Omeprazole by Means of Chiral High Performance Liquid Chromatography (HPLC).

Omeprazole (10.0373 g; 29.058 mmol) was placed in a volumetric flask (500 mL) with 0.1% diethylamine (DEA) in methanol, dissolved by means of sonication, and brought to volume. The solution was injected onto a Waters Delta Prep 4000 HPLC under the following conditions:
Column: Chiralpak AD
Column Dimensions: 20 mm×250 mm; 10 μm particle size
Detection: 280 μm
Flow rate: 10 mL/minute
Mobile Phase: 100% Methanol
Injection Volume: 10 mL

The fractions of the S-omeprazole enantiomer were collected into a flask containing sodium carbonate (10 g). After collection, the sodium carbonate was removed by filtration and the solvent removed by rotary evaporation. The resulting oil was used directly in subsequent experiments (see Examples 1 and 2).

### EXAMPLE 16

Preparation of S-Omeprazole from rac-Omeprazole by Means of Chiral High Performance Liquid Chromatography (HPLC).

Omeprazole (0.7307 g; 2.115 mmol) was dissolved in methanol (37 mL). The solution was injected onto a Waters Delta Prep 4000 HPLC under the following conditions:
Column: Chiralpak AD
Column Dimensions: 20 mm×250 mm; 10 μm particle size
Detection: 280 nm
Flow rate: 10 mL/minute
Mobile Phase: 100% Methanol
Injection Volume: 10 mL

The fractions of the S-omeprazole enantiomer were collected into a flask and the methanol removed by nitrogen purge to produce an oil. The oil was used directly in a subsequent experiment (see Example 3).

### EXAMPLE 17

Recrystallization of Magnesium S-Omeprazole Trihydrate from Dimethylformamide

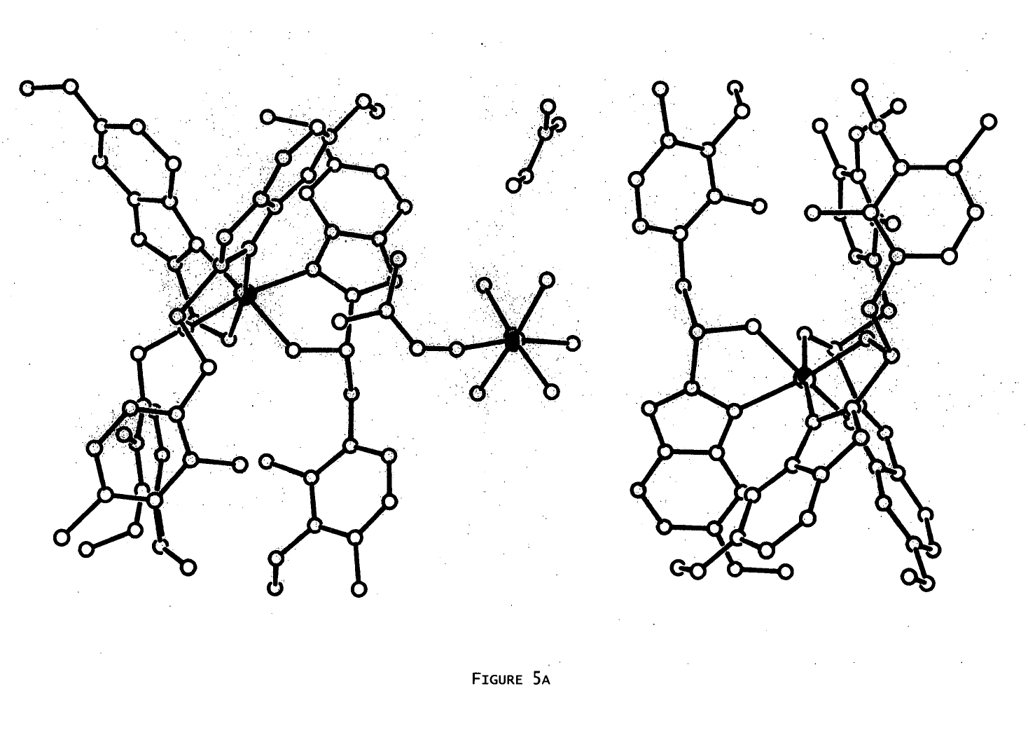

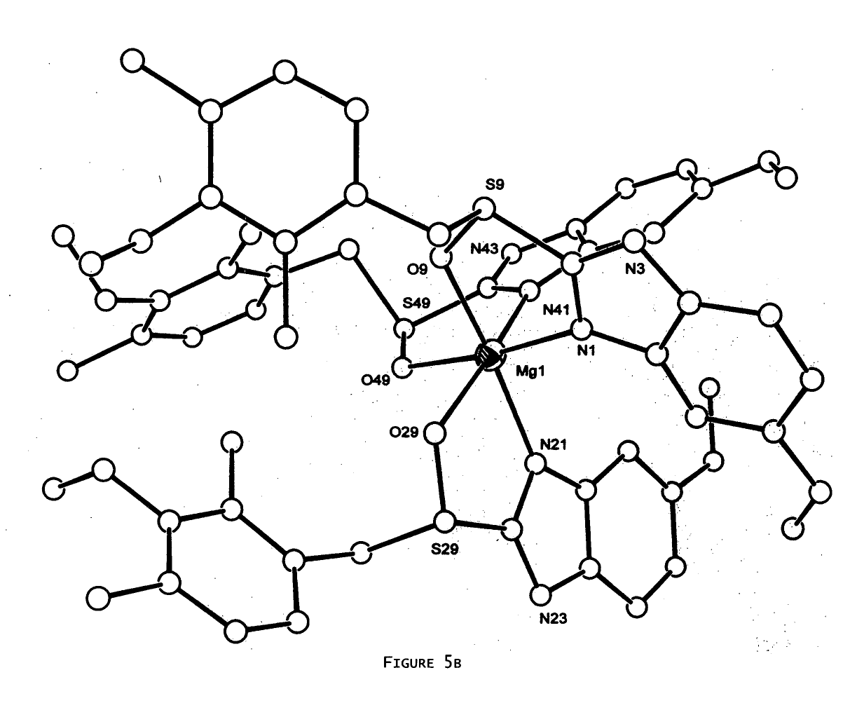

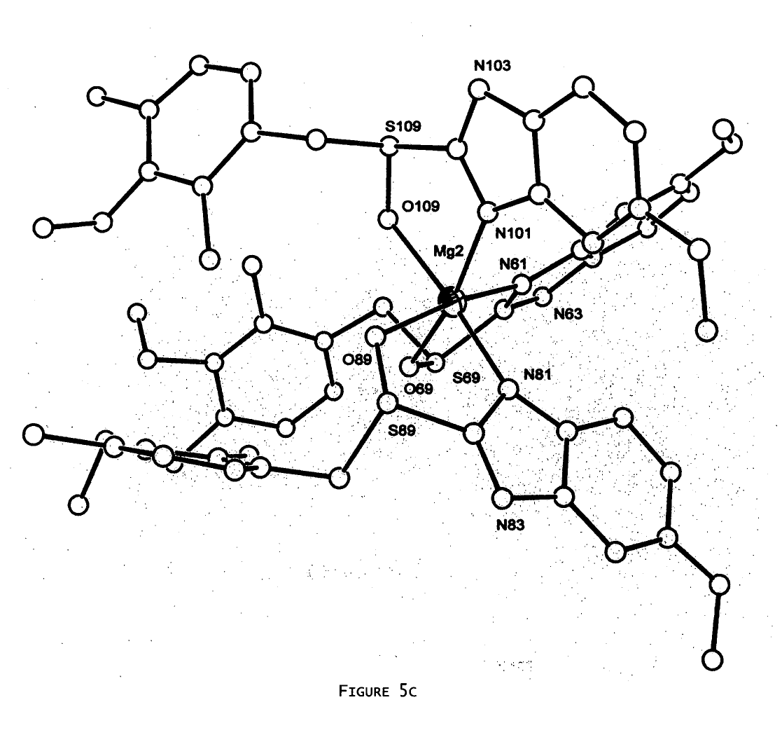

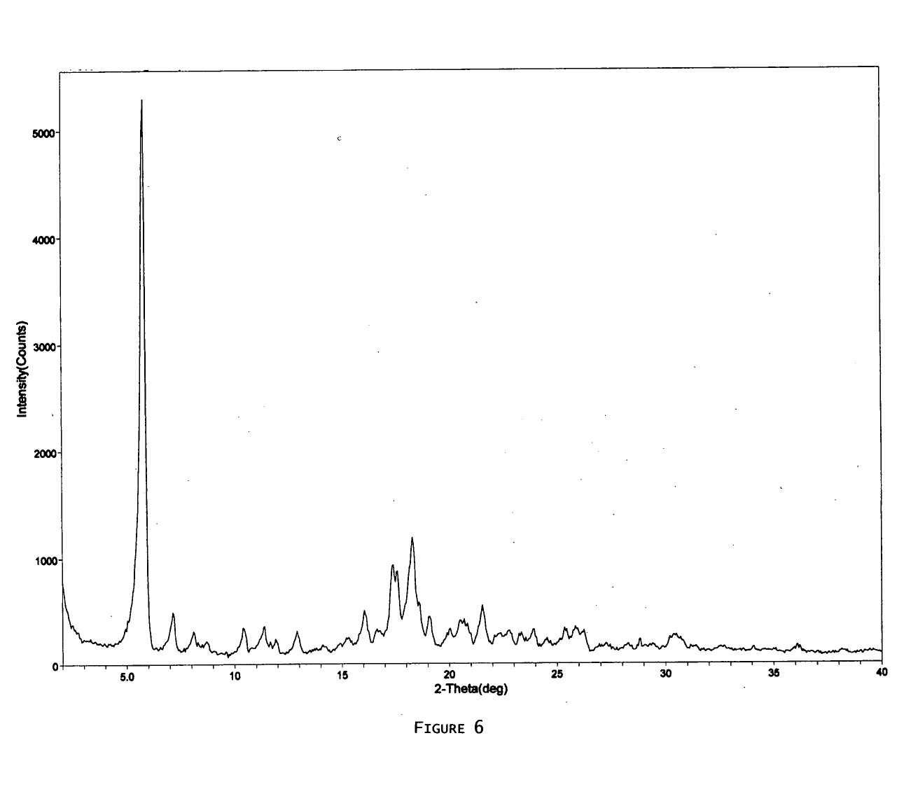

DMF (50 mL) was placed into a 600 mL beaker. Magnesium S-omeprazole was added with stirring until the solution remained slightly cloudy. DMF was added dropwise until the solution clarified. The resulting solution was placed in a crystallization dish and stored under refrigerated conditions for recrystallization. The resulting crystals were characterized by single crystal X-ray analysis, X-ray powder diffraction, Cross-Polarized Magic Angle Spinning solid-state ¹³C NMR spectroscopy (CPMAS), differential scanning calorimetry (melt is from 157.6-172.7° C. followed by degradation), and thermogravimetric analysis (5.36% weight loss from 27.6-115.2° C.). The X-ray crystal structure of magnesium S-omeprazole as determined from this recrystallization is shown in FIGS. 5A, 5B and 5C. The crystalline lattice also contained one uncoordinated DMF molecule. The X-ray powder pattern of the bulk material is substantially the same as that for the computer generated X-ray powder pattern of the single crystal data, confirming that the single crystal was representative of the entire bulk sample. Peak positions and relative intensities for the X-ray powder diffraction are given in Tables 5a and 5b. Resonances for the ¹³C NMR are listed in Table 6. An ORTEP of the molecule is given in FIG. 5A, while an ORTEP of the Δ-[Mg(6-methoxy-5-omeprazolato)₂(5-methoxy-5-omeprazolato)]⁻ and Δ-[Mg(6-methoxy-5-omeprazolato)₃]⁻ anions are given in FIG. 5c. Selected bonds distances and angles are given in Tables 7a and 7b, respectively. The resulting solid product was characterized by means of X-ray powder diffraction. The diffractogram is given in FIG. 6.

TABLE 5a
Positions and intensities of the major peaks in the X-ray
powder diffraction of magnesium S-omeprazole as formed by the
teachings in Example 17.

| d-value/Å | Relative Intensity |
|---|---|
| 15.1 | vs |
| 12.4 | m |
| 10.9 | m |
| 8.5 | w |
| 7.8 | m |
| 6.9 | m |
| 5.5 | m |
| 5.1 | s |
| 5.0 | s |
| 4.8 | s |
| 4.8 | w |
| 4.3 | m |
| 4.1 | m |
| 3.9 | w |
| 3.7 | w |
| 3.5 | w |
| 3.5 | m |
| 3.4 | m |
| 2.9 | m |
| 2.5 | w |

TABLE 5b
Positions and intensities of the major peaks of the computer
generated X-ray powder diffractogram as calculated from the single
crystal data of magnesium S-omeprazole as formed by the teachings
in Example 17.

| | Relative |
|---|---|
| d-value/Å | Intensity |
| 15.3 | vs |
| 12.5 | s |
| 11.0 | s |
| 8.5 | s |
| 7.8 | s |
| 6.9 | s |
| 5.5 | s |
| 5.1 | s |
| 5.1 | vs |
| 4.9 | vs |
| 4.9 | vs |
| 4.7 | s |
| 4.3 | s |
| 4.1 | s |
| 3.9 | s |
| 3.7 | s |
| 3.5 | s |
| 3.5 | s |
| 3.1 | s |
| 3.0 | s |

TABLE 6
Positions of the peaks in the CPMAS spectrum of magnesium
S-omeprazole as formed by the teachings in Example 17.
shift (ppm)

166.6
165.9
164.6
162.6
158.5
157.4
155.5
150.2
148.7
143.9
142.5
138.8
129.8
128.7
127.7
118.4
115.9
112.0
110.4
99.3
97.0
62.5
60.0
59.1
55.1
53.6
35.3
30.9
11.7
11.0
9.6

TABLE 7a
Selected Bond Distances for the Crystalline Product of Example 17.

| Bond | Bond Distance (Å) |
|---|---|
| Mg1—O29 | 2.103(7) |
| Mg1—N21 | 2.156(9) |
| Mg1—N41 | 2.159(7) |
| S9—O9 | 1.516(7) |
| S9—C10 | 1.796(10) |
| S29—C22 | 1.792(11) |
| S49—O49 | 1.518(6) |
| S49—C50 | 1.809(10) |
| O6—C8 | 1.418(17) |
| O14—C18 | 1.382(14) |
| O25—C28 | 1.493(19) |
| O34—C38 | 1.403(17) |
| O46—C46 | 1.376(12) |
| N1—C7A | 1.378(10) |
| N3—C3A | 1.402(13) |
| N11—C12 | 1.355(14) |
| N21—C27A | 1.394(12) |
| N23—C23A | 1.382(12) |
| N31—C32 | 1.384(13) |
| N41—C47A | 1.394(11) |
| N43—C43A | 1.383(11) |
| C3A—C7A | 1.430(15) |
| C7A—C7 | 1.382(15) |
| C12—C13 | 1.389(14) |
| C13—C17 | 1.528(17) |
| C15—C16 | 1.381(16) |
| C23A—C24 | 1.399(15) |
| C24—C25 | 1.360(14) |
| C25—C26 | 1.448(16) |
| Mg1—O49 | 2.150(6) |
| Mg1—O9 | 2.156(7) |
| Mg1—N1 | 2.164(9) |
| S9—C2 | 1.772(10) |
| S29—O29 | 1.518(7) |
| S29—C30 | 1.820(10) |
| S49—C42 | 1.801(9) |
| O6—C6 | 1.368(15) |
| O14—C14 | 1.357(12) |
| O25—C25 | 1.376(14) |
| O34—C34 | 1.412(13) |
| O46—C48 | 1.437(18) |
| N1—C2 | 1.338(13) |
| N3—C2 | 1.341(13) |
| N11—C16 | 1.348(14) |
| N21—C22 | 1.330(11) |
| N23—C22 | 1.326(13) |
| N31—C36 | 1.319(15) |
| N41—C42 | 1.336(11) |
| N43—C42 | 1.321(11) |
| C3A—C4 | 1.382(13) |
| C4—C5 | 1.378(17) |
| C5—C6 | 1.388(18) |
| C6—C7 | 1.363(13) |
| C10—C12 | 1.523(14) |
| C13—C14 | 1.389(15) |
| C14—C15 | 1.409(18) |
| C15—C19 | 1.561(17) |
| C23A—C27A | 1.418(13) |
| C26—C27 | 1.375(16) |
| C32—C33 | 1.380(13) |
| C33—C37 | 1.528(15) |
| C35—C36 | 1.354(15) |
| C43A—C47A | 1.409(13) |
| C44—C45 | 1.354(13) |
| C45—C46 | 1.427(15) |
| C46—C47 | 1.355(14) |
| C50—C52 | 1.53(2) |
| N51—C52 | 1.356(16) |
| C56—C55 | 1.370(17) |
| O54—C54 | 1.438(19) |
| C53—C54 | 1.384(16) |
| C54—C55 | 1.344(19) |
| Mg2—O109 | 2.122(6) |
| Mg2—O69 | 2.153(7) |
| Mg2—N61 | 2.173(8) |
| S69—C62 | 1.800(10) |
| C27A—C27 | 1.401(13) |
| C30—C32 | 1.470(14) |
| C33—C34 | 1.375(15) |
| C34—C35 | 1.420(15) |
| C35—C39 | 1.509(15) |
| C43A—C44 | 1.413(13) |
| C47—C47A | 1.405(13) |
| N51—C56 | 1.319(14) |
| C52—C53 | 1.351(16) |
| O54—C58 | 1.51(5) |
| C53—C57 | 1.55(3) |
| C55—C59 | 1.52(2) |
| Mg2—O89 | 2.111(6) |
| Mg2—N81 | 2.137(7) |
| Mg2—N101 | 2.171(9) |
| S69—O69 | 1.512(7) |
| S69—C70 | 1.828(9) |
| S89—O89 | 1.517(6) |
| S89—C90 | 1.827(10) |
| S109—C102 | 1.762(11) |
| O66—C66 | 1.371(16) |
| O74—C74 | 1.405(11) |
| O86—C86 | 1.391(14) |
| O94—C98 | 1.403(17) |
| O114—C114 | 1.383(12) |
| N61—C62 | 1.337(13) |
| N63—C62 | 1.363(13) |
| N71—C76 | 1.308(14) |
| N81—C87A | 1.357(10) |
| N83—C82 | 1.323(11) |
| N91—C92 | 1.336(14) |
| N101—C102 | 1.351(11) |
| N103—C102 | 1.351(13) |
| N111—C116 | 1.318(14) |
| C17A—C103 | 1.418(14) |
| C63A—C67A | 1.392(14) |
| C64—C65 | 1.370(18) |
| C65—C66 | 1.41(2) |
| C66—C67 | 1.349(15) |
| C70—C72 | 1.489(14) |
| C73—C74 | 1.395(15) |
| C74—C75 | 1.391(17) |
| C75—C79 | 1.516(16) |
| C83A—C87A | 1.412(13) |
| C87A—C87 | 1.374(13) |
| C90—C92 | 1.510(13) |
| S89—C82 | 1.745(9) |
| S109—O109 | 1.534(7) |
| S109—C110 | 1.818(10) |
| O66—C68 | 1.42(2) |
| O74—C78 | 1.408(15) |
| O86—C88 | 1.478(16) |
| O94—C94 | 1.357(13) |
| O106—C106 | 1.414(13) |
| O106—C108 | 1.60(4) |
| O114—C118 | 1.396(13) |
| N61—C67A | 1.403(11) |
| N63—C63A | 1.401(13) |
| N71—C72 | 1.343(13) |
| N81—C82 | 1.376(11) |
| N83—C83A | 1.429(13) |
| N91—C96 | 1.339(12) |
| N101—C17A | 1.382(12) |
| N103—C103 | 1.389(12) |
| N111—C112 | 1.370(12) |
| C17A—C107 | 1.422(13) |
| C63A—C64 | 1.398(14) |
| C67A—C67 | 1.391(14) |
| C72—C73 | 1.369(13) |
| C73—C77 | 1.546(15) |
| C75—C76 | 1.378(14) |
| C83A—C84 | 1.366(13) |
| C84—C85 | 1.330(16) |
| C85—C86 | 1.392(17) |
| C86—C87 | 1.376(14) |
| C93—C94 | 1.385(13) |
| C94—C95 | 1.403(17) |
| C95—C99 | 1.536(14) |
| C104—C105 | 1.384(15) |
| C105—C106 | 1.400(17) |
| C106—C107 | 1.359(16) |
| C110—C112 | 1.476(14) |
| C113—C114 | 1.357(15) |
| C114—C115 | 1.422(15) |
| C115—C119 | 1.524(16) |
| Mg3—O406 | 2.035(7) |
| Mg3—O402 | 2.077(6) |
| Mg3—O404 | 2.102(6) |
| N401—C402 | 1.380(18) |
| C92—C93 | 1.410(15) |
| C93—C97 | 1.530(16) |
| C95—C96 | 1.377(16) |
| C103—C104 | 1.392(16) |
| C112—C113 | 1.401(14) |
| C113—C117 | 1.531(14) |
| C115—C116 | 1.377(15) |
| Mg3—O401 | 2.020(9) |
| Mg3—O405 | 2.069(8) |
| Mg3—O403 | 2.100(7) |
| O401—C401 | 1.250(18) |
| N401—C401 | 1.353(18) |
| N401—C403 | 1.385(18) |
| O503—C503 | 1.159(18) |
| N501—C503 | 1.37(2) |
| N501—C502 | 1.37(2) |
| N501—C501 | 1.46(2) |

TABLE 7b
Selected Bond Angles for the Crystalline Product of Example 17.

| Bond Angle | Angle (deg) |
|---|---|
| O29—Mg1—O49 | 91.4(3) |
| O49—Mg1—N21 | 92.6(3) |
| O49—Mg1—O9 | 87.8(3) |
| O29—Mg1—N41 | 170.2(3) |
| N21—Mg1—N41 | 95.5(3) |
| O29—Mg1—N1 | 93.1(3) |
| N21—Mg1—N1 | 100.9(3) |
| N41—Mg1—N1 | 96.7(3) |
| O9—S9—C10 | 105.9(4) |
| O29—S29—C22 | 103.6(4) |
| C22—S29—C30 | 94.9(5) |
| O49—S49—C50 | 105.4(4) |
| C6—O6—C8 | 116.8(10) |
| C14—O14—C18 | 117.0(9) |
| S29—O29—Mg1 | 120.3(4) |
| C46—O46—C48 | 116.6(10) |
| C2—N1—C7A | 103.2(8) |
| C7A—N1—Mg1 | 138.3(8) |
| C16—N11—C12 | 117.7(10) |
| C22—N21—Mg1 | 116.9(7) |
| C22—N23—C23A | 102.2(8) |
| C42—N41—C47A | 100.1(8) |
| C47A—N41—Mg1 | 140.0(6) |
| N1—C2—N3 | 119.5(9) |
| N3—C2—S9 | 122.3(11) |
| C4—C3A—C7A | 118.6(9) |
| C5—C4—C3A | 117.8(11) |
| C7—C6—O6 | 126.0(12) |
| O6—C6—C5 | 114.9(11) |
| N1—C7A—C3A | 107.0(9) |
| C6—C7—C7A | 119.1(11) |
| O29—Mg1—N21 | 81.4(3) |
| O29—Mg1—O9 | 91.3(3) |
| N21—Mg1—O9 | 172.8(3) |
| O49—Mg1—N41 | 79.4(3) |
| O9—Mg1—N41 | 91.7(3) |
| O49—Mg1—N1 | 166.2(4) |
| O9—Mg1—N1 | 79.0(3) |
| O9—S9—C2 | 102.6(6) |
| C2—S9—C10 | 97.6(4) |
| O29—S29—C30 | 107.0(5) |
| O49—S49—C42 | 103.9(4) |
| C42—S49—C50 | 99.1(5) |
| S9—O9—Mg1 | 121.7(3) |
| C25—O25—C28 | 117.0(12) |
| C38—O34—C34 | 113.7(11) |
| S49—O49—Mg1 | 120.4(3) |
| C2—N1—Mg1 | 118.5(6) |
| C2—N3—C3A | 100.1(10) |
| C22—N21—C27A | 101.9(8) |
| C27A—N21—Mg1 | 139.6(6) |
| C36—N31—C32 | 117.3(10) |
| C42—N41—Mg1 | 119.7(6) |
| C42—N43—C43A | 101.3(7) |
| N1—C2—S9 | 118.1(8) |
| C4—C3A—N3 | 131.3(11) |
| N3—C3A—C7A | 110.0(8) |
| C4—C5—C6 | 123.7(11) |
| C7—C6—C5 | 119.1(11) |
| N1—C7A—C7 | 131.3(11) |
| C7—C7A—C3A | 121.6(9) |
| N11—C12—C10 | 114.6(9) |
| C12—C13—C14 | 116.9(12) |
| C14—C13—C17 | 118.8(11) |
| O14—C14—C15 | 117.6(11) |
| C16—C15—C14 | 119.7(11) |
| C14—C15—C19 | 123.1(12) |
| N23—C22—N21 | 119.2(9) |
| N21—C22—S29 | 117.3(9) |
| N23—C23A—C27A | 108.7(9) |
| C25—C24—C23A | 119.0(11) |
| C24—C25—C26 | 120.5(12) |
| C27—C26—C25 | 120.2(11) |
| N21—C27A—C23A | 108.0(8) |
| C26—C27—C27A | 119.6(11) |
| C33—C32—C30 | 125.4(10) |
| C34—C33—C32 | 117.9(9) |
| C32—C33—C37 | 120.7(10) |
| C33—C34—C35 | 123.1(10) |
| C36—C35—C34 | 112.6(10) |
| C34—C35—C39 | 122.6(12) |
| N11—C12—C13 | 124.7(9) |
| C13—C12—C10 | 120.6(12) |
| C12—C13—C17 | 123.9(10) |
| O14—C14—C13 | 122.8(14) |
| C13—C14—C15 | 119.2(11) |
| C16—C15—C19 | 117.2(16) |
| N11—C16—C15 | 121.8(14) |
| N23—C22—S29 | 123.4(7) |
| N23—C23A—C24 | 129.8(10) |
| C24—C23A—C27A | 121.5(10) |
| C24—C25—O25 | 127.3(12) |
| O25—C25—C26 | 112.2(11) |
| N21—C27A—C27 | 132.9(10) |
| C27—C27A—C23A | 119.1(10) |
| C32—C30—S29 | 110.2(8) |
| C33—C32—N31 | 120.8(9) |
| N31—C32—C30 | 113.9(10) |
| C34—C33—C37 | 121.4(10) |
| C33—C34—O34 | 120.1(10) |
| O34—C34—C35 | 116.7(10) |
| C36—C35—C39 | 124.7(11) |
| N31—C36—C35 | 128.2(11) |
| N43—C42—N41 | 120.7(8) |
| N41—C42—S49 | 115.5(7) |
| N43—C43A—C44 | 132.3(8) |
| C45—C44—C43A | 118.4(9) |
| C47—C46—O46 | 124.9(11) |
| O46—C46—C45 | 113.4(10) |
| N41—C47A—C47 | 128.4(9) |
| C47—C47A—C43A | 122.4(9) |
| C52—C50—S49 | 104.3(9) |
| C56—N51—C52 | 117.3(19) |
| C53—C52—C50 | 122.1(17) |
| N51—C56—C55 | 124.4(17) |
| C52—C53—C54 | 118.9(18) |
| C54—C53—C57 | 119.8(17) |
| C55—C54—O54 | 117.6(14) |
| C54—C55—C56 | 117.4(14) |
| C56—C55—C59 | 118.4(15) |
| N43—C42—S49 | 123.7(7) |
| N43—C43A—C47A | 108.7(8) |
| C47A—C43A—C44 | 119.0(9) |
| C44—C45—C46 | 121.6(9) |
| C47—C46—C45 | 121.5(1) |
| C46—C47—C47A | 117.0(9) |
| N41—C47A—C43A | 109.2(8) |
| C53—C52—N51 | 122(2) |
| N51—C52—C50 | 116.1(1) |
| C54—O54—C58 | 106(3) |
| C52—C53—C57 | 121.3(1) |
| C55—C54—C53 | 120.3(1) |
| C53—C54—O54 | 121.5(1) |
| C54—C55—C59 | 124.2(1) |
| O89—Mg2—O109 | 92.9(2) |
| O109—Mg2—N81 | 174.3(3) |
| O109—Mg2—O69 | 88.9(3) |
| O89—Mg2—N101 | 92.5(3) |
| N81—Mg2—N101 | 97.2(3) |
| O89—Mg2—N61 | 169.9(3) |
| N81—Mg2—N61 | 95.6(3) |
| N101—Mg2—N61 | 97.5(3) |
| O69—S69—C70 | 105.4(4) |
| O89—S89—C82 | 103.3(4) |
| C82—S89—C90 | 97.8(4) |
| O109—S109—C110 | 105.0(4) |
| C66—O66—C68 | 118.8(12) |
| C74—O74—C78 | 115.1(10) |
| S89—O89—Mg2 | 120.4(3) |
| S109—O109—Mg2 | 119.7(4) |
| C62—N61—C67A | 101.7(8) |
| C67A—N61—Mg2 | 139.4(8) |
| C76—N71—C72 | 118.6(9) |
| C87A—N81—Mg2 | 141.6(6) |
| C82—N83—C83A | 102.5(8) |
| C102—N101—C17A | 101.3(8) |
| O89—Mg2—N81 | 81.8(3) |
| O89—Mg2—O69 | 91.1(3) |
| N81—Mg2—O69 | 93.4(3) |
| O109—Mg2—N101 | 80.8(3) |
| O69—Mg2—N101 | 169.2(3) |
| O109—Mg2—N61 | 90.0(3) |
| O69—Mg2—N61 | 79.2(3) |
| O69—S69—C62 | 102.7(5) |
| C62—S69—C70 | 100.0(4) |
| O89—S89—C90 | 106.9(5) |
| O109—S109—C102 | 104.8(4) |
| C102—S109—C110 | 100.2(5) |
| S69—O69—Mg2 | 122.0(3) |
| C86—O86—C88 | 117.8(9) |
| C94—O94—C98 | 115.0(9) |
| C106—O106—C108 | 111.6(15) |
| C114—O114—C118 | 117.1(9) |
| C62—N61—Mg2 | 118.8(6) |
| C62—N63—C63A | 97.7(9) |
| C87A—N81—C82 | 103.2(6) |
| C82—N81—Mg2 | 115.0(5) |
| C92—N91—C96 | 114.2(9) |
| C102—N101—Mg2 | 117.6(7) |
| C17A—N101—Mg2 | 141.0(6) |
| C116—N111—C112 | 117.3(9) |
| N101—C17A—C107 | 128.2(9) |
| N61—C62—N63 | 120.8(9) |
| N63—C62—S69 | 121.8(10) |
| C67A—C63A—N63 | 112.9(9) |
| C65—C64—C63A | 117.0(13) |
| C67—C66—O66 | 124.4(15) |
| O66—C66—C65 | 116.2(12) |
| C67—C67A—N61 | 130.4(11) |
| C66—C67—C67A | 118.4(12) |
| N71—C72—C70 | 115.3(9) |
| C72—C73—C74 | 117.0(10) |
| C74—C73—C77 | 120.8(9) |
| C75—C74—O74 | 117.9(10) |
| C76—C75—C74 | 113.1(10) |
| C74—C75—C79 | 124.2(10) |
| N83—C82—N81 | 117.0(8) |
| N81—C82—S89 | 119.3(6) |
| C84—C83A—N83 | 132.1(10) |
| C85—C84—C83A | 118.9(11) |
| C102—N103—C103 | 102.0(7) |
| N101—C17A—C103 | 109.9(8) |
| C103—C17A—C107 | 121.6(10) |
| N61—C62—S69 | 117.2(8) |
| C67A—C63A—C64 | 119.3(11) |
| C64—C63A—N63 | 127.9(13) |
| C64—C65—C66 | 123.4(12) |
| C67—C66—C65 | 119.3(13) |
| C67—C67A—C63A | 122.6(9) |
| C63A—C67A—N61 | 107.0(10) |
| C72—C70—S69 | 108.3(7) |
| N71—C72—C73 | 121.8(9) |
| C73—C72—C70 | 122.9(10) |
| C72—C73—C77 | 122.2(10) |
| C75—C74—C73 | 122.8(9) |
| C73—C74—O74 | 119.0(11) |
| C76—C75—C79 | 122.7(12) |
| N71—C76—C75 | 126.6(11) |
| N83—C82—S89 | 123.7(8) |
| C84—C83A—C87A | 120.2(10) |
| C87A—C83A—N83 | 107.5(8) |
| C84—C85—C86 | 122.4(10) |
| C87—C86—O86 | 122.7(12) |
| O86—C86—C85 | 117.2(10) |
| N81—C87A—C83A | 109.7(8) |
| C87A—C87—C86 | 118.1(10) |
| N91—C92—C90 | 112.4(10) |
| C94—C93—C92 | 116.7(10) |
| C92—C93—C97 | 123.5(10) |
| O94—C94—C95 | 119.6(10) |
| C96—C95—C94 | 116.4(10) |
| C94—C95—C99 | 122.5(11) |
| N101—C102—N103 | 118.5(9) |
| N103—C102—S109 | 124.6(7) |
| N103—C103—C17A | 108.2(9) |
| C105—C104—C103 | 118.9(11) |
| C107—C106—C105 | 124.4(11) |
| C105—C106—O106 | 111.8(13) |
| C112—C110—S109 | 107.5(8) |
| C87—C86—C85 | 120.1(10) |
| N81—C87A—C87 | 129.9(9) |
| C87—C87A—C83A | 120.3(9) |
| C92—C90—S89 | 109.5(6) |
| N91—C92—C93 | 125.6(9) |
| C93—C92—C90 | 122.0(11) |
| C94—C93—C97 | 119.7(11) |
| O94—C94—C93 | 120.2(12) |
| C93—C94—C95 | 119.9(10) |
| C96—C95—C99 | 121.1(13) |
| N91—C96—C95 | 127.0(11) |
| N101—C102—S109 | 116.9(8) |
| N103—C103—C104 | 132.0(10) |
| C104—C103—C17A | 119.8(10) |
| C104—C105—C106 | 119.7(12) |
| C107—C106—O106 | 123.8(12) |
| C106—C107—C17A | 115.5(10) |
| N111—C112—C113 | 121.4(9) |
| C113—C112—C110 | 124.7(9) |
| C114—C113—C117 | 120.2(10) |
| C113—C114—O114 | 121.5(10) |
| O114—C114—C115 | 116.1(11) |
| C116—C115—C119 | 123.4(11) |
| N111—C116—C115 | 126.8(10) |
| O401—Mg3—O405 | 94.3(4) |
| O401—Mg3—O402 | 85.6(3) |
| O405—Mg3—O402 | 89.2(3) |
| O406—Mg3—O403 | 86.5(3) |
| O402—Mg3—O403 | 95.4(3) |
| O406—Mg3—O404 | 92.1(3) |
| O402—Mg3—O404 | 175.8(4) |
| C401—O401—Mg3 | 174.4(11) |
| C401—N401—C403 | 122.7(17) |
| N111—C112—C110 | 113.5(9) |
| C114—C113—C112 | 118.2(9) |
| C112—C113—C117 | 121.6(10) |
| C113—C114—C115 | 121.9(10) |
| C116—C115—C114 | 114.2(10) |
| C114—C115—C119 | 122.4(11) |
| O401—Mg3—O406 | 174.2(4) |
| O406—Mg3—O405 | 90.5(3) |
| O406—Mg3—O402 | 91.2(3) |
| O401—Mg3—O403 | 89.0(4) |
| O405—Mg3—O403 | 174.5(3) |
| O401—Mg3—O404 | 91.3(3) |
| O405—Mg3—O404 | 88.2(3) |
| O403—Mg3—O404 | 87.4(3) |
| C401—N401—C402 | 125(3) |
| C402—N401—C403 | 112(3) |
| C503—N501—C502 | 116.7(16) |
| C503—N501—C501 | 118.6(19) |
| C502—N501—C501 | 124.5(19) |
| O503—C503—N501 | 125.8(19) |

### EXAMPLE 18

Recrystallization of Magnesium S-Omeprazole Trihydrate from Dimethylformamide

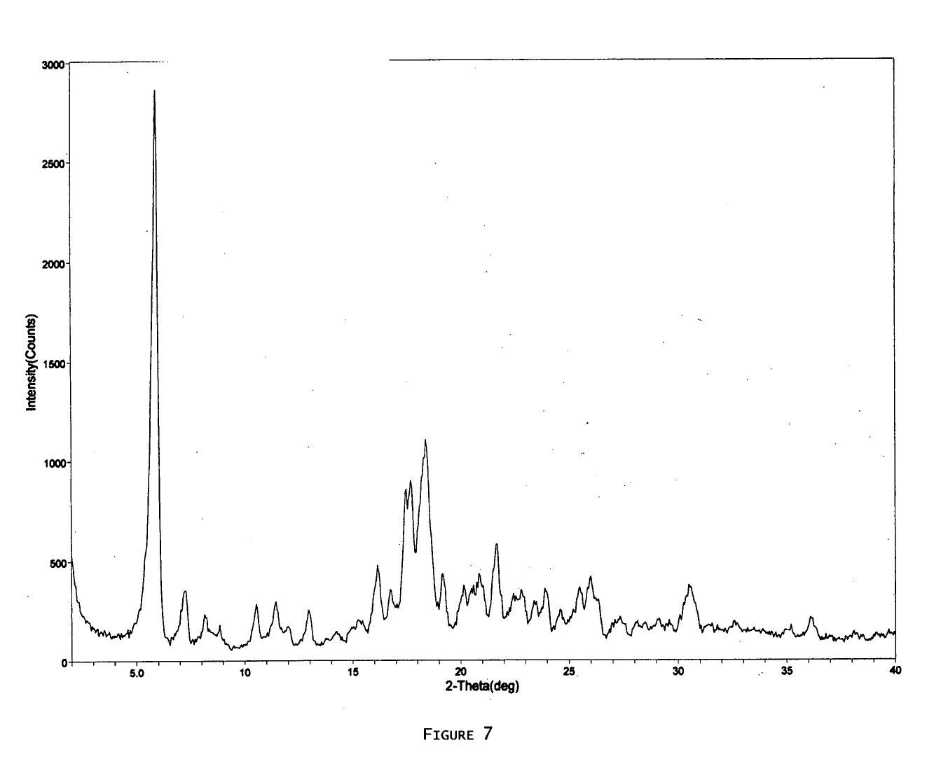

DMF (30 mL) was placed into a 100 mL beaker. Magnesium S-omeprazole was added with stirring until the solution remained slightly cloudy. DMF was added dropwise until the solution clarified. The resulting solution was placed in a crystallization dish and stored in a cabinet for recrystallization. The resulting crystals were characterized by X-ray powder diffraction, differential scanning calorimetry (melt from 163.7-174.5° C., followed by degradation), and thermogravimetric analysis (6.27% weight loss from 26.3-116.4° C.). Peak positions and relative intensities for the X-ray powder diffraction are given in Table 8. The resulting solid product was characterized by means of X-ray powder diffraction. The diffractogram is given in FIG. 7.

TABLE 8
Positions and intensities of the major peaks in the X-ray powder
diffraction of magnesium S-omeprazole as formed by the
teachings in Example 18.

| d-value/Å | Relative Intensity |
|---|---|
| 15.1 | vs |
| 12.3 | m |
| 10.9 | m |
| 8.5 | m |
| 7.8 | m |
| 6.9 | m |
| 5.5 | m |
| 5.1 | s |
| 5.0 | s |
| 4.9 | vs |
| 4.7 | m |
| 4.3 | m |
| 4.1 | m |
| 3.9 | m |
| 3.7 | m |
| 3.5 | m |
| 3.4 | m |
| 3.4 | m |
| 2.9 | m |
| 2.5 | w |

### EXAMPLE 19

Recrystallization of Magnesium S-Omeprazole Trihydrate from Dimethylformamide

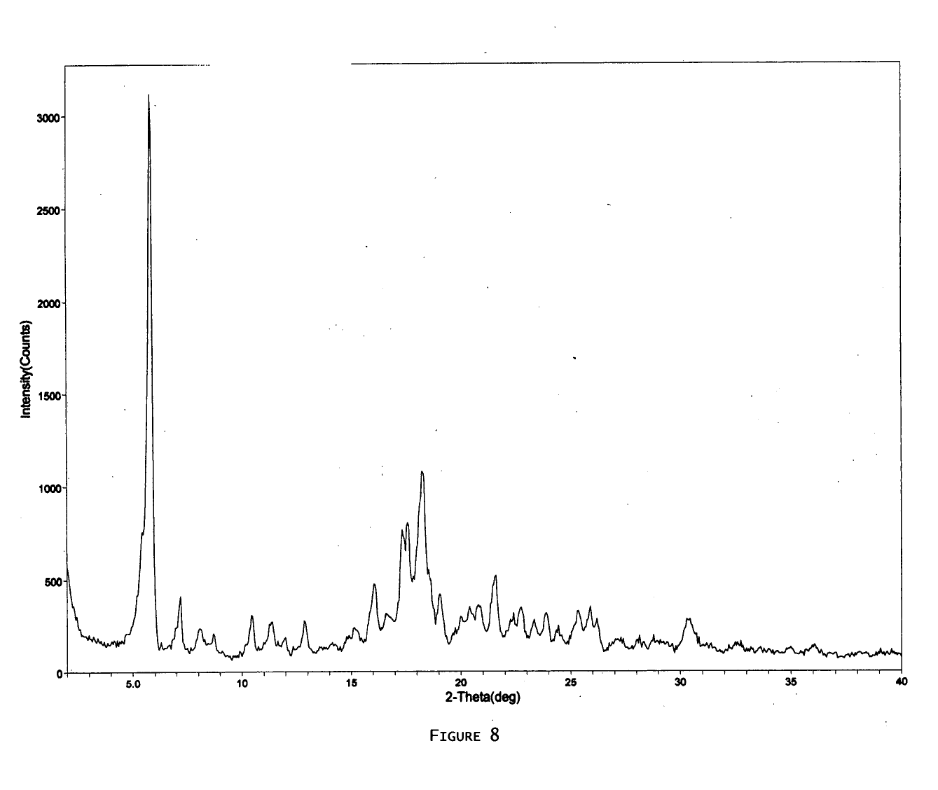

DMF (30 mL) was placed into a 100 mL beaker. Magnesium S-omeprazole was added with stirring until the solution remained slightly cloudy. DMF was added dropwise until the solution clarified. The resulting solution was placed in a crystallization dish and stored in a cabinet for recrystallization. The resulting crystals were characterized by X-ray powder diffraction, differential scanning calorimetry (melt from 162.2-175.3° C. followed by degradation), and thermogravimetric analysis (3.75% weight loss from 23.0-116.3° C.). Peak positions and relative intensities for the X-ray powder diffraction are given in Table 9. The resulting solid product was characterized by means of X-ray powder diffraction. The diffractogram is given in FIG. 8.

TABLE 9
Positions and intensities of the major peaks in the X-ray powder
diffraction of magnesium S-omeprazole as formed by the
teachings in Example 19.

| | Relative |
|---|---|
| d-value/Å | Intensity |
| 14.9 | vs |
| 12.2 | m |
| 10.8 | m |
| 8.4 | m |
| 7.7 | m |
| 6.8 | m |
| 5.5 | m |
| 5.1 | s |
| 5.0 | s |
| 4.8 | vs |
| 4.6 | m |
| 4.4 | m |
| 4.3 | m |
| 4.1 | s |
| 4.0 | m |
| 3.9 | m |
| 3.8 | w |
| 3.7 | m |
| 3.5 | m |
| 3.4 | m |
| 2.9 | m |
| 2.5 | m |

### EXAMPLE 20

Recrystallization of Magnesium S-Omeprazole Dihydrate from Dimethylformamide

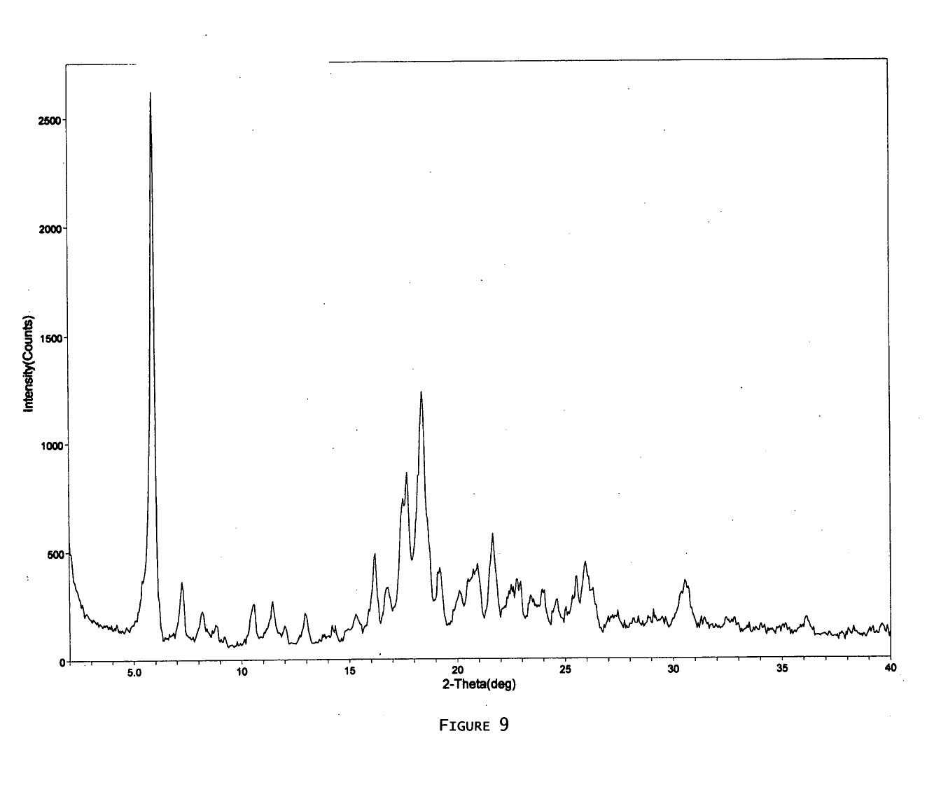

Dimethylformamide (DMF) (15 mL) was placed in a 150 mL beaker. Magnesium S-omeprazole was added with stirring until the solution was slightly cloudy. Additional DMF was added dropwise until the solution clarified. The resulting solution was placed in a petrie dish and stored under refrigerated conditions to recrystallize. The crystalline material obtained was characterized by X-ray powder diffraction, thermogravimetric analysis (4.95% weight loss from 27.4-115.5° C.) and differential scanning calorimetry (melt from 161.2-170.9° C. followed by degradation). Peak positions and relative intensities for the X-ray powder diffraction are given in Table 10. The resulting solid product was characterized by means of X-ray powder diffraction. The diffractogram is given in FIG. 9.

TABLE 10
Positions and intensities of the major peaks in the X-ray
powder diffraction of magnesium S-omeprazole as formed by
the teachings in Example 20.

| | Relative |
|---|---|
| d-value/Å | Intensity |
| 14.9 | vs |
| 12.2 | m |
| 10.8 | m |
| 8.4 | m |
| 7.7 | m |
| 6.8 | m |
| 5.5 | s |
| 5.1 | s |
| 5.0 | s |
| 4.8 | vs |
| 4.6 | m |
| 4.4 | w |
| 4.2 | m |
| 4.1 | s |
| 3.9 | m |
| 3.8 | w |
| 3.7 | m |
| 3.5 | m |
| 3.4 | s |
| 2.9 | m |
| 2.5 | w |

### EXAMPLE 21

Recrystallization of Magnesium S-Omeprazole Trihydrate from Methanol

Methanol (300 mL) was placed in a 600 mL beaker. Magnesium S-omeprazole was slowly added to the solution with stirring until the solution remained slightly cloudy. Methanol was added dropwise until the solution clarified. The resulting solution was placed in a crystallization dish and stored under refrigeration for recrystallization. The resulting crystals were characterized by X-ray powder diffraction, differential scanning calorimetry (no endotherm detected, sample degrades after approximately 175° C.), and thermogravimetric analysis (7.93% weight loss from 24.6-115.3° C.). The resulting powder pattern for this material indicated that it was amorphous with no crystalline character.

### EXAMPLE 22

Recrystallization of Magnesium S-Omeprazole Trihydrate from Methanol Using an Acetone Chamber

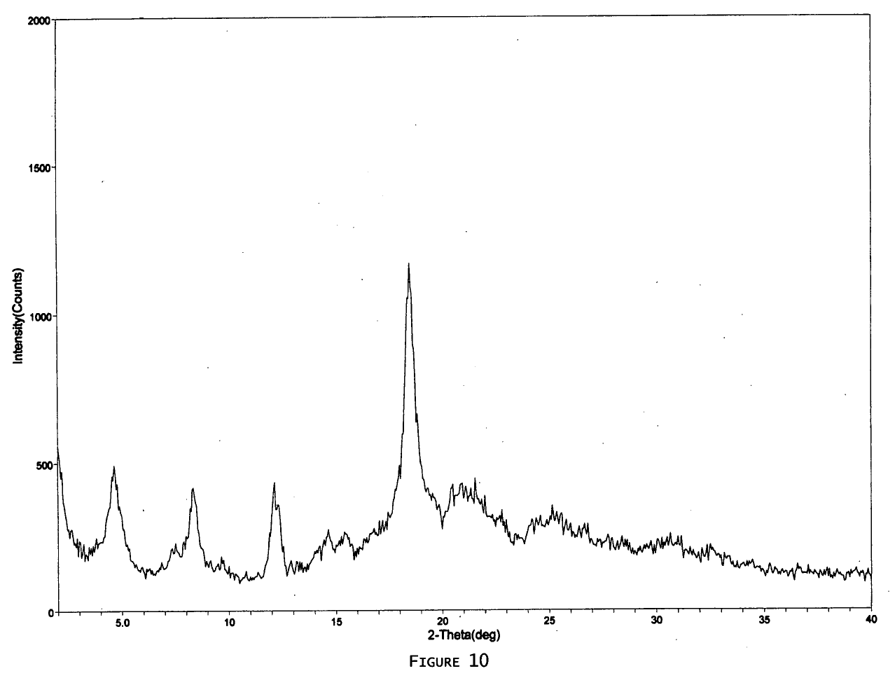

Methanol (200 mL) was placed into a 400 mL beaker. Magnesium S-omeprazole was added with stirring until the solution remained slightly cloudy. Methanol was the added dropwise until the solution clarified. Approximately 3 mL of water was added to the solution. Half of the methanolic magnesium S-omeprazole solution was placed into an open petrie dish. This dish was then placed inside of a larger petrie dish. Acetone was added to the outside petrie dish creating an acetone chamber for vapor diffusion recrystallization. The larger petrie dish was then covered and placed in a cabinet at room temperature to recrystallize. The level of acetone was periodically checked and replenished as needed during the recrystallization process. The resulting crystals were characterized by X-ray powder diffraction, differential scanning calorimetry (broad endotherm from 57.8-91.3° C., sample degrades after approximately 175° C.), and thermogravimetric analysis (8.90% weight loss from 24.4-115.1° C.). Peak positions and relative intensities for the X-ray powder diffraction are given in Table 11. The resulting solid product was characterized by means of X-ray powder diffraction. The diffractogram is given in FIG. 10.

TABLE 11
Positions and intensities of the major peaks in the X-ray
powder diffraction of magnesium S-omeprazole as formed
by the teachings in Example 22.

| | Relative |
|---|---|
| d-value/Å | Intensity |
| 19.0 | vs |
| 12.0 | m |
| 10.6 | vs |
| 9.2 | m |
| 7.3 | vs |
| 6.0 | m |
| 5.8 | m |
| 4.8 | vs |
| 4.4 | s |
| 4.1 | s |
| 3.5 | m |
| 3.3 | m |
| 2.9 | m |
| 2.8 | m |

### EXAMPLE 23

Recrystallization of Magnesium S-Omeprazole Trihydrate from a Methanol/Acetone Solution

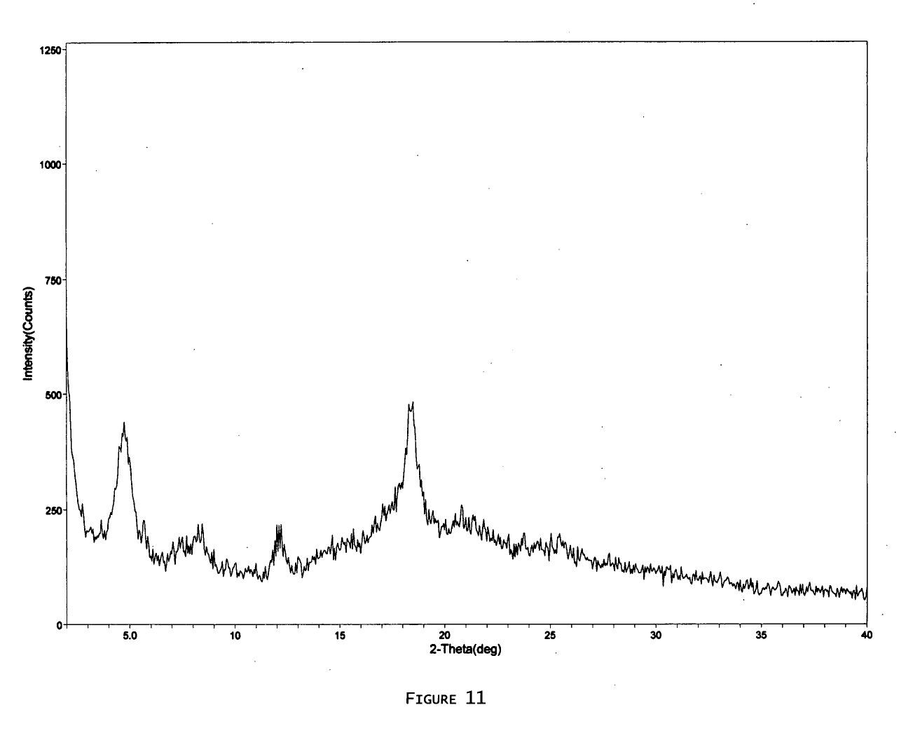

Methanol (200 mL) was placed into a 400 mL beaker. Magnesium S-omeprazole was added with stirring until the solution remained slightly cloudy. Methanol was added dropwise until the solution clarified. Approximately 3 mL of water was added to the solution. Twenty-five mL of the methanolic magnesium S-omeprazole solution was placed into clean 150 mL beaker. Approximately 20 mL acetone was added and the solution was placed in a cabinet at room temperature to recrystallize. The resulting crystals were characterized by X-ray powder diffraction, differential scanning calorimetry (minor endotherm from 58.5-83.5° C., sample degrades after approximately 175° C.), and thermogravimetric analysis (8.61% weight loss from 25.2-115.3° C.). Peak positions and relative intensities for the X-ray powder diffraction are given in Table 12. The resulting solid product was characterized by means of X-ray powder diffraction. The diffractogram is given in FIG. 11.

TABLE 12
Positions and intensities of the major peaks in the X-ray
powder diffraction of magnesium S-omeprazole as formed
by the teachings in Example 23.

| d-value/Å | Relative Intensity |
|---|---|
| 18.6 | vs |
| 12.0 | s |
| 10.5 | vs |
| 7.8 | vs |
| 4.8 | vs |
| 4.8 | vs |
| 4.3 | s |
| 3.5 | s |

### EXAMPLE 24

Recrystallization of Magnesium S-Omeprazole Trihydrate from a Methanol/Acetone Solution

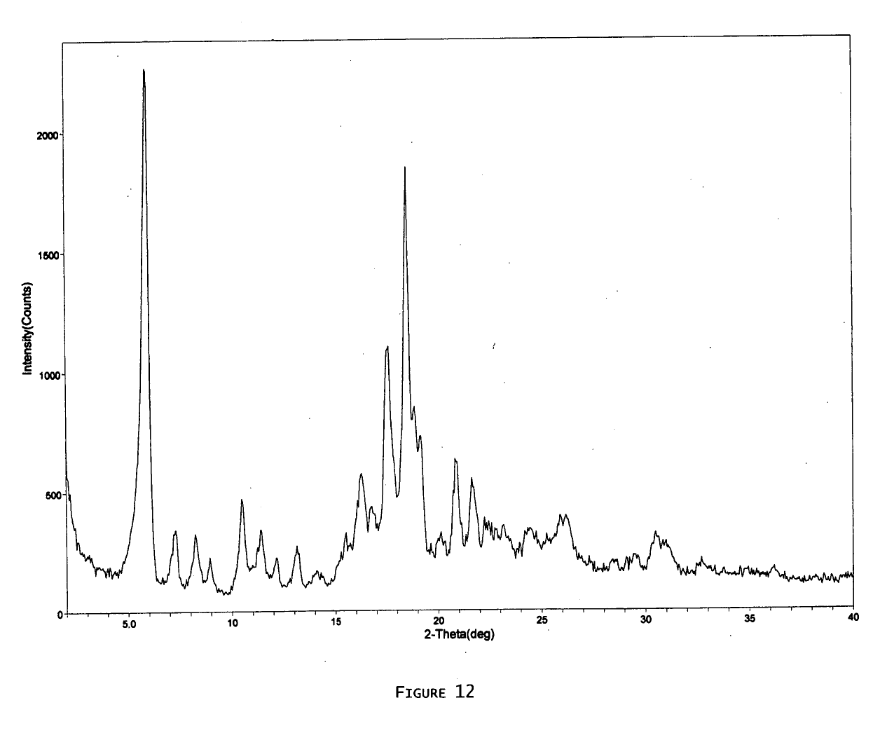

Magnesium S-omeprazole (7.6 g, 11 mmol) was placed in a 100 mL beaker. Methanol (ca. 10 mL) was added with stirring. An additional 10 mL aliquot of methanol was added and the resulting solution was allowed to evaporate back down to approximately 10 mL. Acetone (25 mL) was added with stirring. The resulting solution was covered with a watchglass and allowed to stand for about one hour after which a white solid had precipitated. The solution was decanted from the solid material, which was dried. The resulting crystals were characterized by X-ray powder diffraction, differential scanning calorimetry (minor endotherm from 99.9-118.6° C., sample degrades after approximately 175° C.), and thermogravimetric analysis (6.23% weight loss from 22.2-115.1° C.). The X-ray powder pattern for the recrystallized sample is substantially the same as that for the crystal grown from DMF in Example 17. Peak positions and relative intensities for the X-ray powder diffraction are given in Table 13. The resulting solid product was characterized by means of X-ray powder diffraction. The diffractogram is given in FIG. 12.

TABLE 13
Positions and intensities of the major peaks in the X-ray
powder diffraction of magnesium S-omeprazole as formed
by the teachings in Example 24.

| | Relative |
|---|---|
| d-value/Å | Intensity |
| 14.8 | vs |
| 12.1 | s |
| 10.7 | m |
| 8.4 | s |
| 7.8 | m |
| 6.7 | m |
| 5.7 | m |
| 5.4 | s |
| 5.0 | vs |
| 4.8 | vs |
| 4.7 | vs |
| 4.6 | s |
| 4.4 | m |
| 4.3 | s |
| 4.1 | s |
| 4.0 | w |
| 3.8 | w |
| 3.6 | m |
| 3.4 | m |
| 2.9 | m |
| 2.9 | m |
| 2.5 | w |

### EXAMPLE 25

Recrystallization of Magnesium S-Omeprazole Trihydrate from a Methanol/Acetone Solution

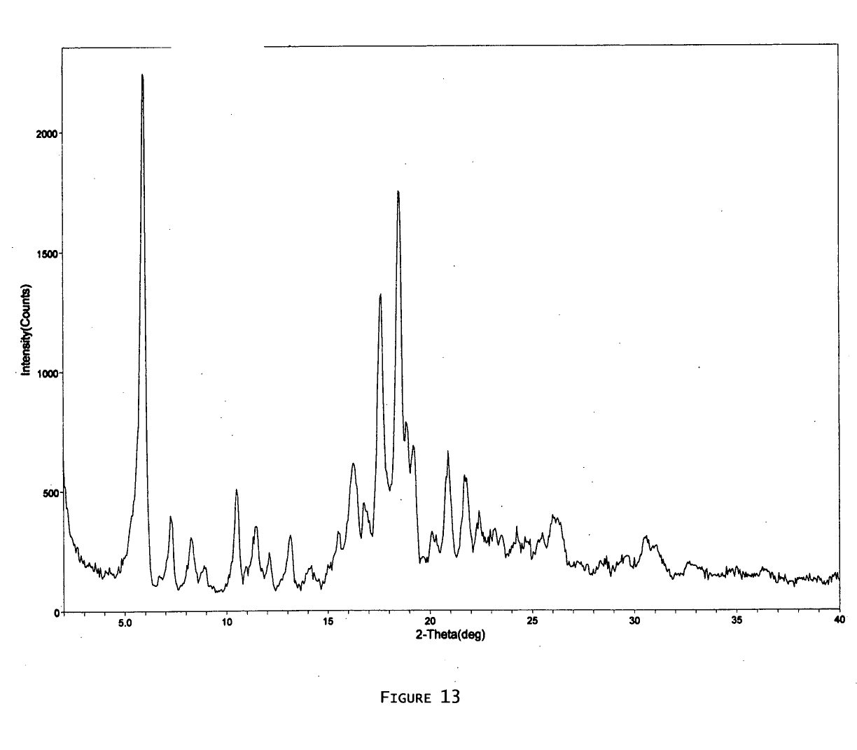

Magnesium S-omeprazole (14.5 g, 20.4 mmol) was placed in a 250 mL beaker. Methanol (ca. 40 mL) was added with stirring. The resulting solution was allowed to evaporate to approximately 20 mL. Acetone (50 mL) was added with stirring. The resulting solution was covered and allowed to stand overnight after which a white solid had precipitated. The solution was decanted from the solid material, which was dried. The resulting crystals were characterized by X-ray powder diffraction, CPMAS, differential scanning calorimetry (minor, broad endotherm from 112.7-150.4° C., sample degrades after approximately 175° C.), and thermogravimetric analysis (4.50% weight loss from 30.2-115.3° C.). The X-ray powder pattern for the recrystallized sample is substantially the same as that for the crystal grown from DMF in Example 17. Peak positions and relative intensities for the X-ray powder diffraction are given in Table 14. Resonances for the ¹³C NMR are listed in Table 15. The resulting solid product was characterized by means of X-ray powder diffraction. The diffractogram is given in FIG. 13.

TABLE 14
Positions and intensities of the major peaks in the X-ray
powder diffraction of magnesium S-omeprazole as formed
by the teachings in Example 25.

| d-value/Å | Relative Intensity |
|---|---|
| 14.8 | vs |
| 12.2 | s |
| 10.7 | m |
| 8.4 | s |
| 7.7 | m |
| 7.3 | m |
| 6.7 | m |
| 5.7 | m |
| 5.5 | s |
| 5.3 | m |
| 5.0 | vs |
| 4.8 | vs |
| 4.7 | s |
| 4.6 | s |
| 4.4 | m |
| 4.3 | s |
| 4.1 | s |
| 4.0 | m |
| 3.8 | w |
| 3.7 | m |
| 3.4 | m |
| 2.9 | m |
| 2.9 | m |
| 2.5 | w |

TABLE 15
Positions of the peaks in the CPMAS spectrum of magnesium
S-omeprazole as formed by the teachings in Example 25.
shift (ppm)

167.1
166.6
164.5
156.8
155.9
149.2
144.0
141.6
138.8
129.9
128.7
127.2
118.6
117.5
116.0
112.3
111.4
97.8
96.4
62.5
59.4
56.0
54.6
53.1
30.1
13.2
11.8
10.0

### EXAMPLE 26

Recrystallization of Magnesium S-Omeprazole Trihydrate from a Methanol/Acetone/Water Solution

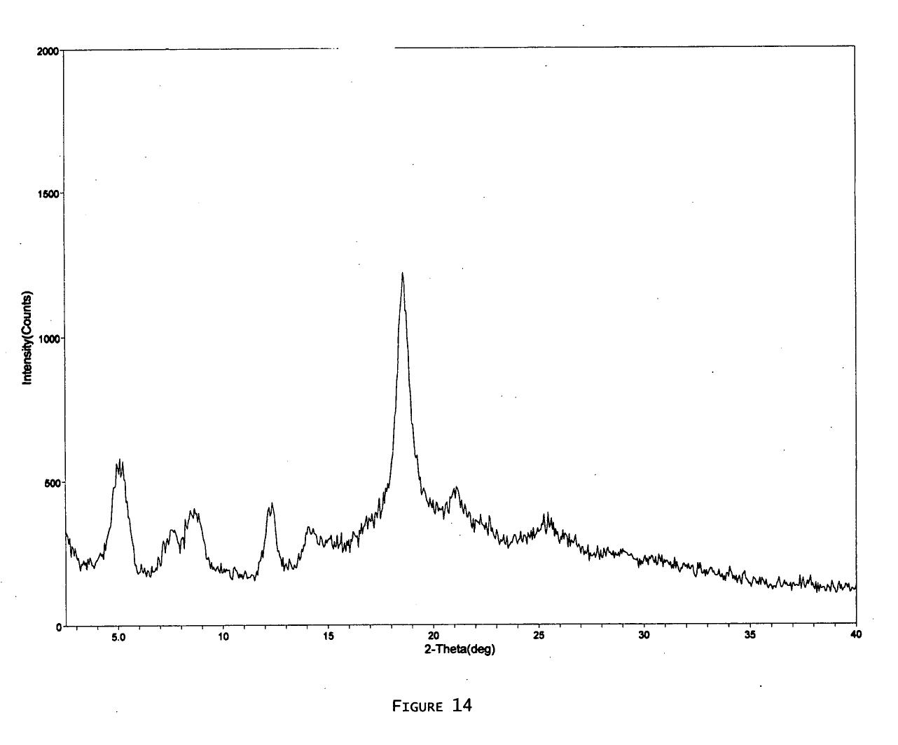

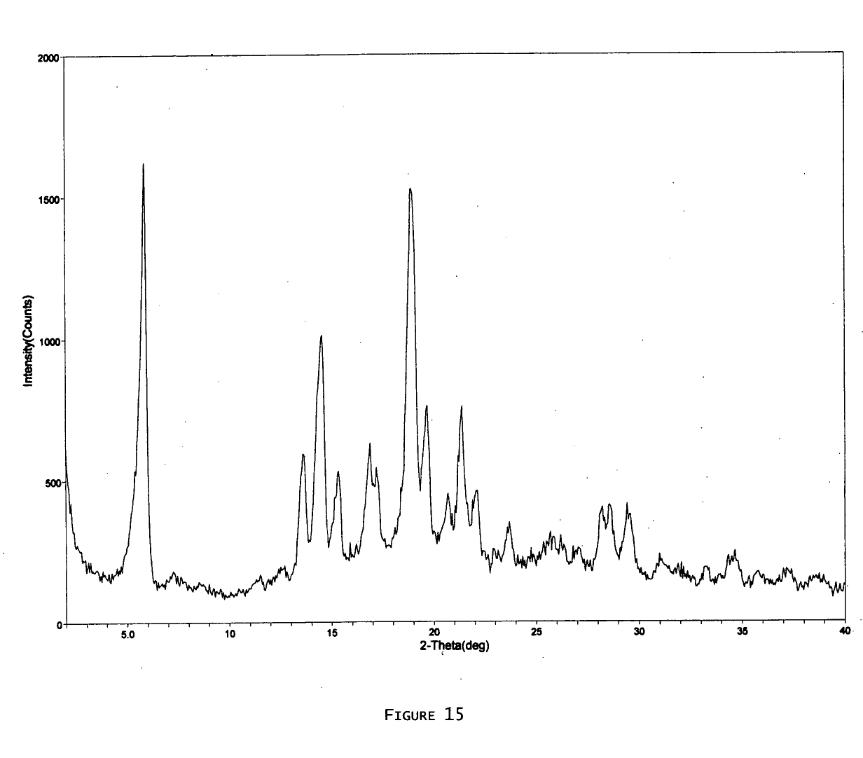

Methanol (20 mL) was placed into a 50 mL beaker. Magnesium S-omeprazole (8.6 g, 12 mmol) was added with stirring resulting in a very thick, slightly opaque solution. This was placed in a cabinet to evaporate down to approximately 7 mL. Water (5 mL) and acetone (30 mL) were mixed together and the methanolic solution of magnesium S-omeprazole solution was added to this solution with stirring. The resulting solution was allowed to stand for one hour, after which a solid material had precipitated from the solution. The solid material was filtered off and dried in a vacuum oven set at 40.0° C. The resulting crystals were characterized by X-ray powder diffraction, differential scanning calorimetry (minor endotherm from 59.1-72.5° C., minor endotherm from 151.3-175.7° C. followed by degradation), and thermogravimetric analysis (4.68% weight loss from 36.3-114.8° C.). Peak positions and relative intensities for the X-ray powder diffraction are given in Tables 16 and 17. The resulting solid product was characterized by means of X-ray powder diffraction. The diffractograms are given in FIGS. 14 and 15.

TABLE 16
Positions and intensities of the major peaks in the X-ray
powder diffraction of magnesium S-omeprazole as formed
by the teachings in Example 26 before drying.

| d-value/Å | Relative Intensity |
|---|---|
| 17.3 | vs |
| 11.7 | m |
| 10.3 | s |
| 7.2 | vs |
| 6.3 | s |
| 4.8 | vs |
| 4.2 | s |
| 3.5 | s |
| 3.1 | m |

TABLE 17
Positions and intensities of the major peaks in the X-ray
powder diffraction of magnesium S-omeprazole as formed
by the teachings in Example 26 after drying.

| d-value/Å | Relative Intensity |
|---|---|
| 15.0 | vs |
| 12.2 | m |
| 7.7 | w |
| 7.1 | w |
| 6.5 | vs |
| 6.1 | vs |
| 5.8 | s |
| 5.2 | vs |
| 5.2 | s |
| 4.7 | vs |
| 4.5 | s |
| 4.3 | m |
| 4.2 | vs |
| 4.0 | s |
| 3.8 | m |
| 3.5 | m |
| 3.4 | m |
| 3.3 | m |
| 3.2 | s |
| 3.0 | s |
| 2.9 | m |
| 2.7 | w |
| 2.6 | m |
| 2.5 | w |
| 2.4 | m |
| 2.3 | m |

### EXAMPLE 27

Recrystallization of Magnesium S-Omeprazole Trihydrate from an Ethanol/Acetone Solution

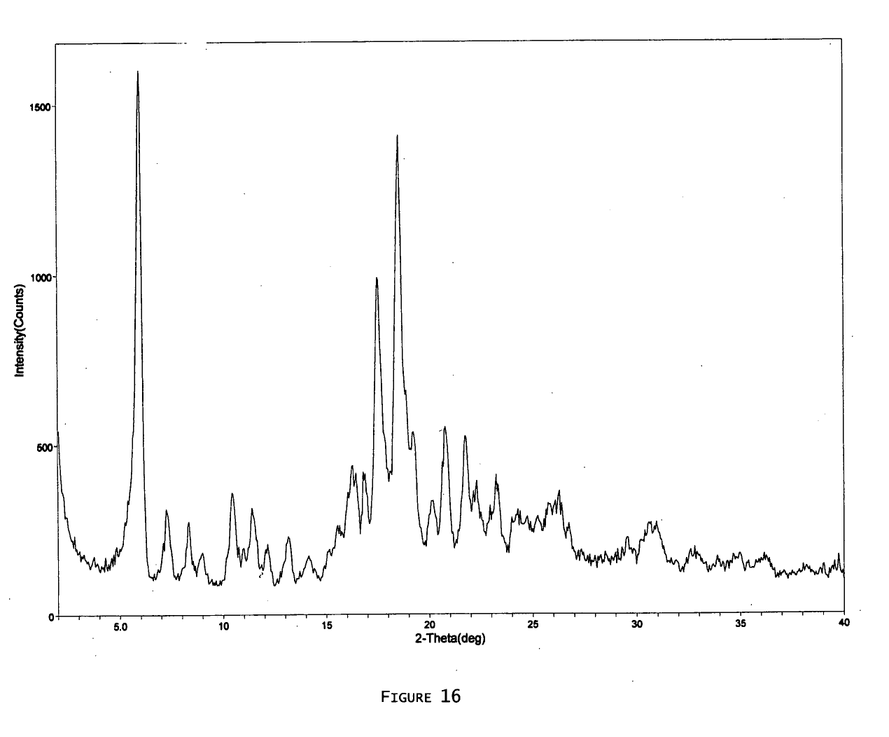

Magnesium S-omeprazole (7.6 g, 11 mmol) was placed in a 600 mL beaker. Absolute ethanol (ca. 200 mL) was added with stirring. The resulting solution was allowed to evaporate back down to approximately 100 mL. Acetone (100 mL) was added with stirring. The resulting solution was covered and allowed to stand overnight after which a white solid had precipitated. The solution was decanted from the solid material, which was dried. The resulting crystals were characterized by X-ray powder diffraction, differential scanning calorimetry, no endotherm detected, sample degrades after approximately 175° C.), and thermogravimetric analysis (6.16% weight loss from 27.2-115.3° C.). The X-ray powder pattern for the recrystallized sample is substantially the same as that for the crystal grown from DMF in Example 17. Peak positions and relative intensities for the X-ray powder diffraction are given in Table 18. The resulting solid product was characterized by means of X-ray powder diffraction. The diffractogram is given in FIG. 16.

TABLE 18
Positions and intensities of the major peaks in the X-ray
powder diffraction of magnesium S-omeprazole as formed by
the teachings in Example 27.

| | Relative |
|---|---|
| d-value/Å | Intensity |
| 14.7 | vs |
| 12.2 | s |
| 10.6 | s |
| 8.5 | s |
| 7.8 | s |
| 7.3 | m |
| 6.7 | m |
| 5.7 | m |
| 5.4 | m |
| 5.3 | m |
| 5.1 | vs |
| 4.8 | vs |
| 4.7 | s |
| 4.6 | s |
| 4.4 | m |
| 4.3 | s |
| 4.1 | s |
| 4.0 | m |
| 3.8 | s |
| 3.7 | m |
| 3.4 | m |
| 2.9 | m |
| 2.9 | m |
| 2.5 | m |

### EXAMPLE 28

Recrystallization of Magnesium S-Omeprazole Trihydrate from an Ethanol/Acetone Solution

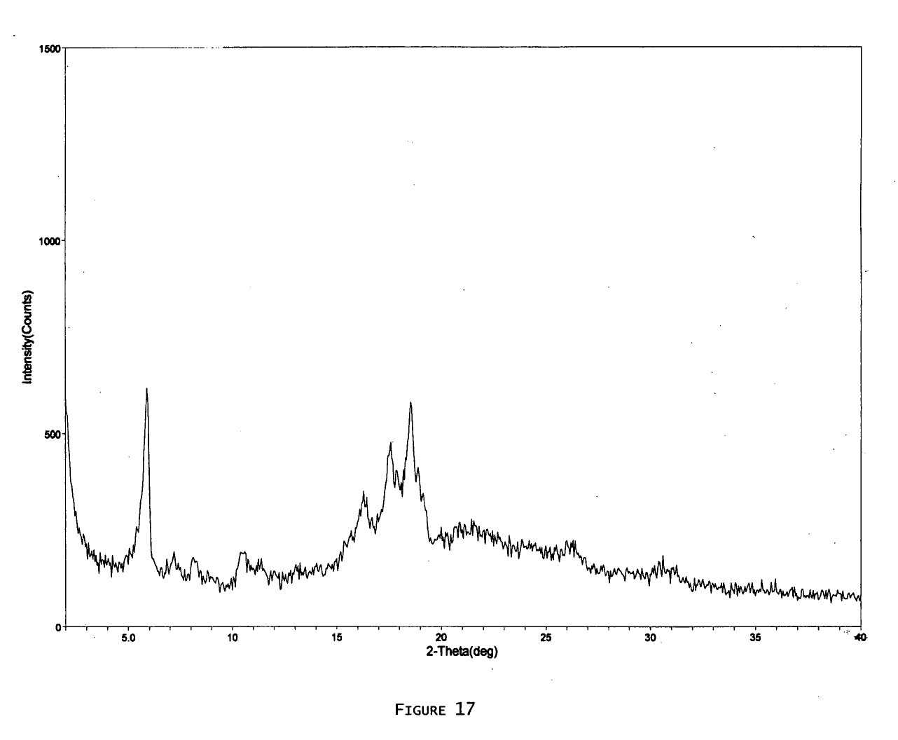

Absolute ethanol (200 mL) was placed into a 400 mL beaker. Magnesium S-omeprazole was added with stirring until the solution remained slightly cloudy. Absolute ethanol was added dropwise until the solution clarified. Twenty-five mL of the ethanolic magnesium S-omeprazole solution was placed into a clean 150 mL beaker. Approximately 20 mL acetone was added and the solution was placed in a cabinet at room temperature to recrystallize. The resulting crystals were characterized by X-ray powder diffraction, differential scanning calorimetry (no endotherm detected, sample degrades after approximately 175° C.), and thermogravimetric analysis (7.83% from 26.5-115.1° C.). Peak positions and relative intensities for the X-ray powder diffraction are given in Table 19. The resulting solid product was characterized by means of X-ray powder diffraction. The diffractogram is given in FIG. 17.

TABLE 19
Positions and intensities of the major peaks in the X-ray
powder diffraction of magnesium S-omeprazole as formed
by the teachings in Example 28

| d-value/Å | Relative Intensity |
|---|---|
| 15.0 | vs |
| 12.3 | s |
| 10.9 | s |
| 8.4 | s |
| 7.8 | m |
| 5.4 | s |
| 5.0 | vs |
| 4.8 | vs |
| 4.7 | vs |
| 4.1 | s |
| 3.4 | s |
| 2.9 | s |

### EXAMPLE 29

Recrystallization of Magnesium S-Omeprazole Trihydrate from Ethanol using an Acetone Chamber

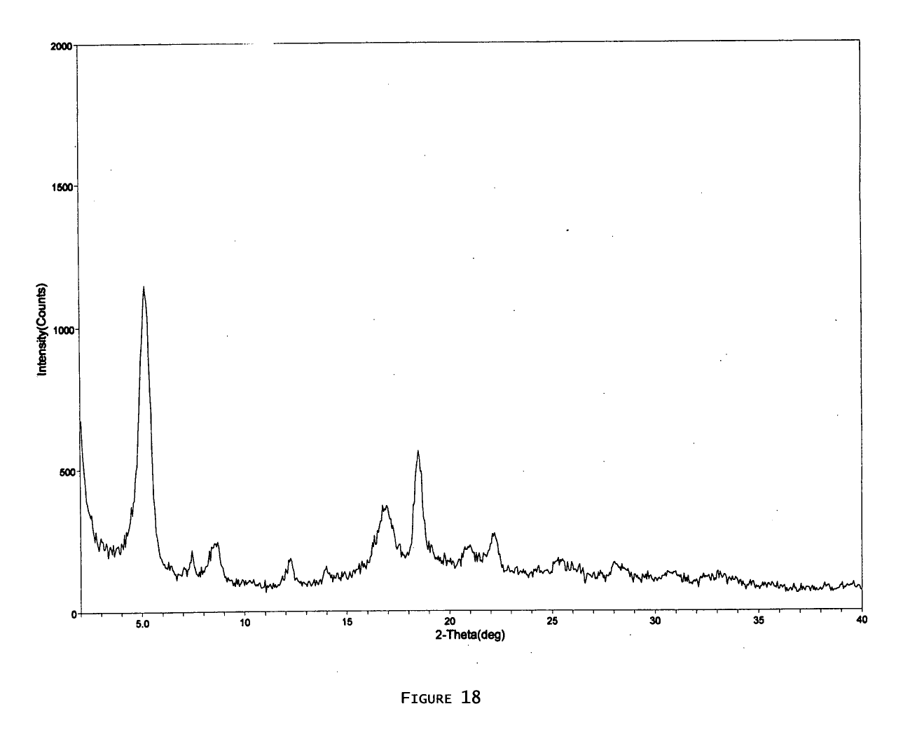

Absolute ethanol (200 mL) was placed into a 400 mL beaker. Magnesium S-omeprazole was added with stirring until the solution remained slightly cloudy. Absolute ethanol was added dropwise until the solution clarified. One half of the ethanolic magnesium S-omeprazole solution was placed into an open petrie dish. This dish was then placed inside of a larger petrie dish. Acetone was added to the outside petrie dish creating an acetone chamber for vapor diffusion recrystallization. The larger petrie dish was then covered and placed in a cabinet at room temperature to recrystallize. The level of acetone was periodically checked and replenished as needed during the recrystallization process. The resulting crystals were characterized by X-ray powder diffraction, CPMAS, differential scanning calorimetry (no endotherm detected, sample degrades after approximately 175° C.), and thermogravimetric analysis (9.92% from 22.3-115.3° C.). Peak positions and relative intensities for the X-ray powder diffraction are given in Table 20. Resonances for the ¹³C NMR are listed in Table 21. The resulting solid product was characterized by means of X-ray powder diffraction. The diffractogram is given in FIG. 18.

TABLE 20
Positions and intensities of the major peaks in the X-ray
powder diffraction of magnesium S-omeprazole as formed
by the teachings in Example 29

| d-value/Å | Relative Intensity |
|---|---|
| 17.0 | vs |
| 11.9 | m |
| 10.2 | s |
| 7.2 | m |
| 6.3 | m |
| 5.3 | s |
| 4.8 | vs |
| 4.2 | m |
| 4.0 | s |
| 3.5 | m |
| 3.2 | m |
| 2.7 | m |

TABLE 21
Positions of the peaks in the CPMAS spectrum of magnesium
S-omeprazole as formed by the teachings in Example 29.
shift (ppm)

164.0
154.6
149.8
146.6
142.6
140.2
136.8
126.3
116.2
111.5
95.9
58.1
52.5
10.5
7.8

### EXAMPLE 30

Recrystallization of Magnesium S-Omeprazole Trihydrate from Ethanol Using an Acetone Chamber

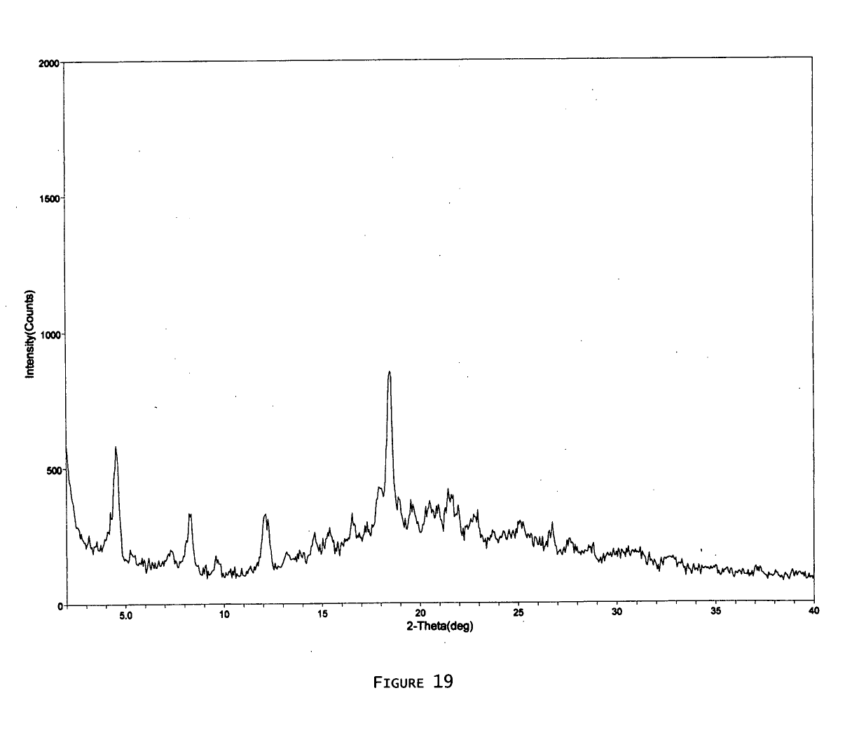

Absolute ethanol (175 mL) was placed into a 400 mL beaker. Magnesium S-omeprazole was added with stirring until the solution remained slightly cloudy. Absolute ethanol was added dropwise until the solution clarified. The ethanolic magnesium S-omeprazole solution was placed into an open petrie dish (100 mm diameter). This dish was then placed inside of a larger petrie dish (150 mm diameter). Acetone was added to the outside petrie dish creating an acetone chamber for vapor diffusion recrystallization. The larger petrie dish was then covered and placed in a cabinet at room temperature to recrystallize. The level of acetone was periodically checked and replenished as needed during the recrystallization process. The resulting crystals were characterized by X-ray powder diffraction, differential scanning calorimetry (minor, broad endotherm from 57.1-76.7° C., sample degrades after approximately 175° C.), and thermogravimetric analysis (11.29% from 28.5-115.2° C.). Peak positions and relative intensities for the X-ray powder diffraction are given in Table 22. The resulting solid product was characterized by means of X-ray powder diffraction. The diffractogram is given in FIG. 19.

TABLE 22
Positions and intensities of the major peaks in the X-ray
powder diffraction of magnesium S-omeprazole as formed
by the teachings in Example 30

| d-value/Å | Relative Intensity |
|---|---|
| 19.4 | vs |
| 12.1 | m |
| 10.7 | vs |
| 9.2 | s |
| 7.3 | vs |
| 6.1 | s |
| 5.8 | s |
| 5.4 | s |
| 5.0 | vs |
| 4.8 | vs |
| 4.7 | s |
| 4.5 | s |
| 4.1 | s |
| 3.9 | s |
| 3.5 | s |
| 3.3 | s |
| 3.2 | m |
| 3.1 | m |
| 2.7 | m |

### EXAMPLE 31

Recrystallization of Magnesium S-Omeprazole Trihydrate from Dimethylsulfoxide

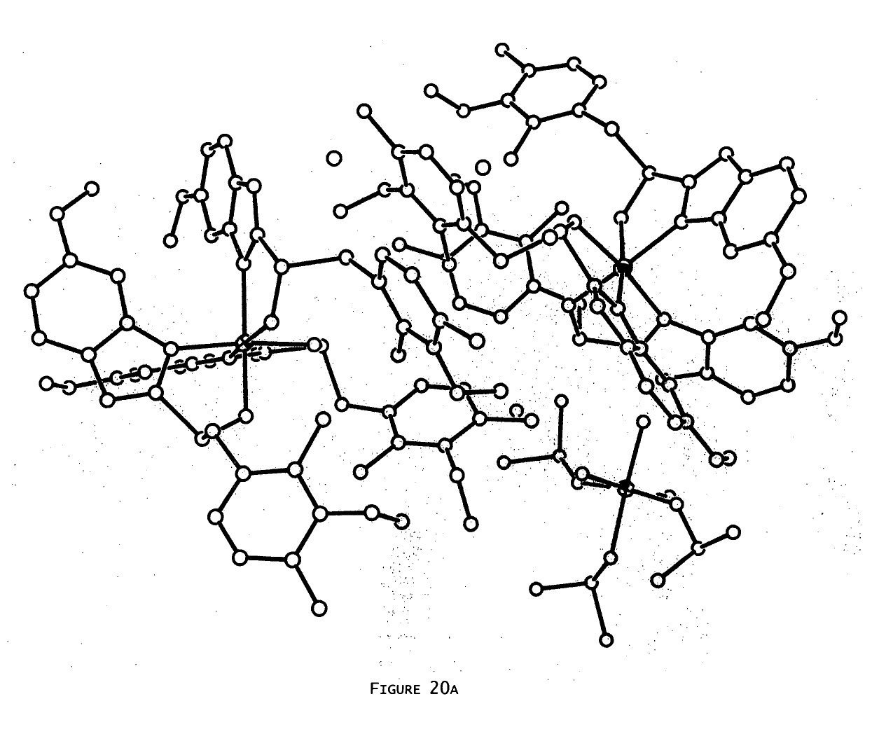

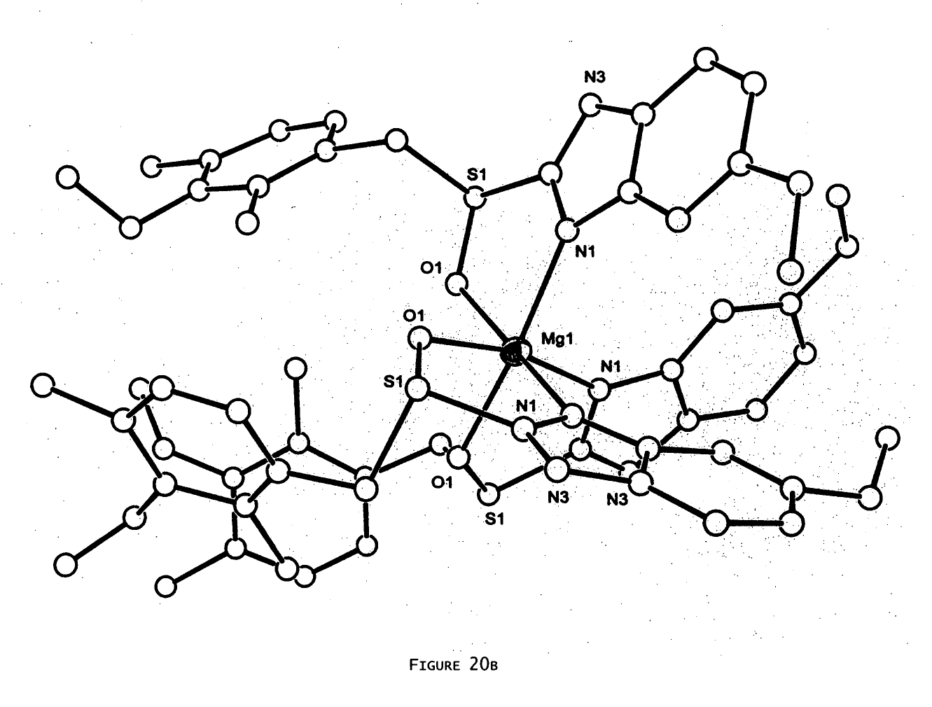

Dimethylsulfoxide (DMSO) (15 mL) was placed into a 25 mL beaker. Magnesium S-omeprazole was added with stirring until the solution remained slightly cloudy. DMSO was added dropwise until the solution clarified. The resulting solution was placed in a petrie dish and stored at room temperature for recrystallization. The resulting crystals were characterized using single crystal X-ray analysis. The X-ray crystal structure of magnesium S-omeprazole as determined from this recrystallization is shown in FIGS. 20A and 20B. The crystalline lattice also contained three uncoordinated water molecules, of which two were partially occupied. A powder pattern was generated from the single crystal data and the results are tabulated in Table 23a. Selected bond distances and angles are given in Tables 23b and 23c, respectively.

TABLE 23a
Positions and intensities of the major peaks in the computer
generated X-ray powder diffraction pattern of magnesium
S-omeprazole as formed by the teachings in Example 31.

| d-value/Å | Relative Intensity |
|---|---|
| 13.7 | vs |
| 9.4 | s |
| 8.7 | s |
| 8.0 | s |
| 7.2 | s |
| 6.2 | s |
| 5.8 | vs |
| 5.2 | vs |
| 5.0 | vs |
| 5.0 | vs |
| 4.9 | vs |
| 4.6 | vs |
| 4.6 | s |
| 4.5 | s |
| 4.4 | s |
| 4.3 | s |
| 4.1 | s |
| 4.0 | vs |
| 3.9 | s |
| 3.9 | vs |
| 3.7 | vs |
| 3.6 | vs |
| 3.5 | vs |
| 3.4 | s |
| 3.3 | s |
| 3.1 | s |
| 3.0 | s |

TABLE 23b
Bond Distances for the Crystalline Product of Example 31.

| Bond | Bond Distance (Å) |
|---|---|
| N11—C21 | 1.350(5) |
| N11—C81 | 1.379(4) |
| N11—Mg(1) | 2.154(4) |
| C21—N31 | 1.326(4) |
| C21—S11 | 1.786(3) |
| N31—C91 | 1.381(5) |
| C41—C51 | 1.357(5) |
| C41—C91 | 1.426(5) |
| C51—C61 | 1.409(5) |
| C61—C71 | 1.365(5) |
| C61—O21 | 1.397(5) |
| C71—C81 | 1.391(5) |
| C81—C91 | 1.402(5) |
| C101—C111 | 1.499(5) |
| C101—S11 | 1.808(4) |
| C111—N121 | 1.350(5) |
| C111—C161 | 1.376(5) |
| N121—C131 | 1.316(5) |
| C131—C141 | 1.392(5) |
| C141—C151 | 1.398(5) |
| C141—C181 | 1.517(6) |
| C151—O31 | 1.362(5) |
| C151—C161 | 1.402(5) |
| C161—C201 | 1.505(5) |
| C171—O21 | 1.414(6) |
| C191—O31 | 1.371(6) |
| O11—S11 | 1.509(3) |
| O11—Mg(1) | 2.089(4) |
| N12—C22 | 1.350(5) |
| N12—C82 | 1.381(4) |
| N12—Mg(1) | 2.170(4) |
| C22—N32 | 1.321(4) |
| C22—S12 | 1.779(4) |
| N32—C92 | 1.381(5) |
| C42—C52 | 1.360(5) |
| C42—C92 | 1.419(5) |
| C52—C62 | 1.393(5) |
| C62—C72 | 1.370(5) |
| C62—O22 | 1.394(5) |
| C72—C82 | 1.389(5) |
| C82—C92 | 1.402(5) |
| C102—C112 | 1.495(5) |
| C102—S12 | 1.812(4) |
| C112—N122 | 1.354(5) |
| C112—C162 | 1.386(5) |
| N122—C132 | 1.319(5) |
| C132—C142 | 1.387(5) |
| C142—C152 | 1.402(5) |
| C142—C182 | 1.523(5) |
| C152—O32 | 1.379(5) |
| C152—C162 | 1.388(5) |
| C162—C202 | 1.512(5) |
| C172—O22 | 1.431(6) |
| C192—O32 | 1.419(5) |
| O12—S12 | 1.512(3) |
| O12—Mg(1) | 2.132(4) |
| N13—C23 | 1.345(4) |
| N13—C83 | 1.383(4) |
| N13—Mg(1) | 2.139(4) |
| C23—N33 | 1.327(4) |
| C23—S13 | 1.794(3) |
| N33—C93 | 1.388(4) |
| C43—C53 | 1.373(5) |
| C43—C93 | 1.424(5) |
| C53—C63 | 1.394(5) |
| C63—C73 | 1.366(5) |
| C63—O23 | 1.400(5) |
| C73—C83 | 1.391(4) |
| C83—C93 | 1.395(5) |
| C103—C113 | 1.496(4) |
| C103—S13 | 1.824(4) |
| C113—N123 | 1.348(4) |
| C113—C163 | 1.390(4) |
| N123—C133 | 1.312(5) |
| C133—C143 | 1.378(5) |
| C143—C153 | 1.412(5) |
| C143—C183 | 1.511(5) |
| C153—O33 | 1.374(5) |
| C153—C163 | 1.390(4) |
| C163—C203 | 1.505(5) |
| C173—O23 | 1.418(6) |
| C193—O33 | 1.401(5) |
| O13—S13 | 1.513(3) |
| O13—Mg(1) | 2.147(4) |
| N14—C24 | 1.347(5) |
| N14—C84 | 1.380(4) |
| N14—Mg(2) | 2.160(4) |
| C24—N34 | 1.323(4) |
| C24—S14 | 1.783(3) |
| N34—C94 | 1.383(4) |
| C44—C54 | 1.366(5) |
| C44—C94 | 1.422(5) |
| C54—C64 | 1.394(5) |
| C64—C74 | 1.367(5) |
| C64—O24 | 1.397(5) |
| C74—C84 | 1.390(5) |
| C84—C94 | 1.400(5) |
| C104—C114 | 1.499(4) |
| C104—S14 | 1.808(4) |
| C114—N124 | 1.345(5) |
| C114—C164 | 1.391(5) |
| N124—C734 | 1.320(5) |
| C734—C144 | 1.385(5) |
| C144—C154 | 1.405(5) |
| C144—C184 | 1.515(5) |
| C154—O34 | 1.379(4) |
| C154—C164 | 1.384(4) |
| C164—C204 | 1.504(5) |
| C174—O24 | 1.423(6) |
| C194—O34 | 1.408(5) |
| O14—S14 | 1.513(3) |
| O14—Mg(2) | 2.157(4) |
| N15—C25 | 1.343(4) |
| N15—C85 | 1.382(4) |
| N15—Mg(2) | 2.148(4) |
| C25—N35 | 1.327(5) |
| C25—S15 | 1.781(4) |
| N35—C95 | 1.381(5) |
| C45—C55 | 1.361(5) |
| C45—C95 | 1.420(5) |
| C55—C65 | 1.404(5) |
| C65—C75 | 1.365(5) |
| C65—O25 | 1.399(5) |
| C75—C85 | 1.393(5) |
| C85—C95 | 1.400(5) |
| C105—C115 | 1.505(5) |
| C105—S15 | 1.820(4) |
| C115—N125 | 1.351(5) |
| C115—C165 | 1.385(4) |
| N125—C135 | 1.315(5) |
| C135—C145 | 1.386(5) |
| C145—C155 | 1.404(5) |
| C145—C185 | 1.514(5) |
| C155—O35 | 1.379(4) |
| C155—C165 | 1.389(4) |
| C165—C205 | 1.506(5) |
| C175—O25 | 1.421(6) |
| C195—O35 | 1.421(5) |
| O15—S15 | 1.520(3) |
| O15—Mg(2) | 2.104(4) |
| N16—C26 | 1.338(5) |
| N16—C86 | 1.386(4) |
| N16—Mg(2) | 2.137(4) |
| C26—N36 | 1.332(4) |
| C26—S16 | 1.791(4) |
| N36—C96 | 1.384(5) |
| C46—C56 | 1.365(5) |
| C46—C96 | 1.420(5) |
| C56—C66 | 1.397(5) |
| C66—C76 | 1.363(5) |
| C66—O26 | 1.403(5) |
| C76—C86 | 1.391(5) |
| C86—C96 | 1.402(5) |
| C106—C116 | 1.490(5) |
| C106—S16 | 1.808(4) |
| C116—N126 | 1.351(5) |
| C116—C166 | 1.395(5) |
| N126—C136 | 1.327(5) |
| C136—C146 | 1.389(5) |
| C146—C156 | 1.409(5) |
| C146—C186 | 1.519(5) |
| C156—O36 | 1.377(4) |
| C156—C166 | 1.391(5) |
| C166—C206 | 1.507(5) |
| C176—O26 | 1.421(6) |
| C196—O36 | 1.412(6) |
| O16—S16 | 1.506(3) |
| O16—Mg(2) | 2.107(4) |
| Mg(3)—O(5) | 2.051(5) |
| Mg(3)—O(6) | 2.058(6) |
| Mg(3)—O(8) | 2.060(6) |
| Mg(3)—O(4) | 2.067(5) |
| Mg(3)—O(9) | 2.076(6) |
| Mg(3)—O(7) | 2.109(5) |
| O(7)—S(2) | 1.500(5) |
| S(2)—C(31) | 1.766(6) |
| S(2)—C(32) | 1.792(6) |
| O(8)—S(3) | 1.498(5) |
| S(3)—C(33) | 1.773(7) |
| S(3)—C(34) | 1.782(7) |
| O(9)—S(4) | 1.480(5) |
| S(4)—C(35) | 1.781(7) |
| S(4)—C(36) | 1.786(7) |

TABLE 23c
Bond Distances for the Crystalline Product of Example 31.

| Bond Angle | Angle (deg) |
|---|---|
| C21—N11—C81 | 101.4(3) |
| C21—N11—Mg(1) | 116.4(2) |
| C81—N11—Mg(1) | 142.0(3) |
| N31—C21—N11 | 119.6(3) |
| N31—C21—S11 | 123.5(3) |
| N11—C21—S11 | 116.8(3) |
| C21—N31—C91 | 100.4(3) |
| C51—C41—C91 | 117.8(4) |
| C41—C51—C61 | 121.9(4) |
| C71—C61—O21 | 124.1(4) |
| C71—C61—C51 | 121.3(4) |
| O21—C61—C51 | 114.6(4) |
| C61—C71—C81 | 117.6(4) |
| N11—C81—C71 | 130.0(4) |
| N11—C81—C91 | 108.0(3) |
| C71—C81—C91 | 122.1(3) |
| N31—C91—C81 | 110.5(3) |
| N31—C91—C41 | 130.2(4) |
| C81—C91—C41 | 119.2(4) |
| C111—C101—S11 | 109.6(3) |
| N121—C111—C161 | 123.7(4) |
| N121—C111—C101 | 113.0(3) |
| C161—C111—C101 | 123.1(4) |
| C131—N121—C111 | 117.7(4) |
| N121—C131—C141 | 124.8(4) |
| C131—C141—C151 | 115.7(4) |
| C131—C141—C181 | 121.9(4) |
| C151—C141—C181 | 122.3(4) |
| O31—C151—C141 | 121.4(4) |
| O31—C151—C161 | 117.2(4) |
| C141—C151—C161 | 120.6(4) |
| C111—C161—C151 | 116.5(4) |
| C111—C161—C201 | 123.9(4) |
| C151—C161—C201 | 119.1(4) |
| S11—O11—Mg(1) | 120.1(2) |
| C61—O21—C171 | 117.7(4) |
| C151—O31—C191 | 121.1(5) |
| O11—S11—C21 | 103.6(2) |
| O11—S11—C101 | 106.3(2) |
| C21—S11—C101 | 98.3(2) |
| C22—N12—C82 | 102.1(3) |
| C22—N12—Mg(1) | 117.2(2) |
| C82—N12—Mg(1) | 139.0(3) |
| N32—C22—N12 | 119.1(3) |
| N32—C22—S12 | 123.8(3) |
| N12—C22—S12 | 117.1(3) |
| C22—N32—C92 | 100.8(3) |
| C52—C42—C92 | 117.6(4) |
| C42—C52—C62 | 122.1(4) |
| C72—C62—C52 | 121.7(4) |
| C72—C62—O22 | 123.3(4) |
| C52—C62—O22 | 114.9(4) |
| C62—C72—C82 | 117.2(4) |
| N12—C82—C72 | 130.6(4) |
| N12—C82—C92 | 107.4(3) |
| C72—C82—C92 | 122.0(3) |
| N32—C92—C82 | 110.7(3) |
| N32—C92—C42 | 129.9(4) |
| C82—C92—C42 | 119.4(4) |
| C112—C102—S12 | 110.4(3) |
| N122—C112—C162 | 123.9(3) |
| N122—C112—C102 | 112.9(4) |
| C162—C112—C102 | 123.3(4) |
| C132—N122—C112 | 117.1(4) |
| N122—C132—C142 | 125.0(4) |
| C132—C142—C152 | 116.1(4) |
| C132—C142—C182 | 121.8(4) |
| C152—C142—C182 | 122.0(4) |
| O32—C152—C162 | 119.4(4) |
| O32—C152—C142 | 119.6(3) |
| C162—C152—C142 | 120.9(3) |
| C112—C162—C152 | 116.8(4) |
| C112—C162—C202 | 122.6(4) |
| C152—C162—C202 | 120.6(4) |
| S12—O12—Mg(1) | 121.6(2) |
| C62—O22—C172 | 116.4(4) |
| C152—O32—C192 | 115.3(4) |
| O12—S12—C22 | 103.1(2) |
| O12—S12—C102 | 105.5(2) |
| C22—S12—C102 | 97.9(2) |
| C23—N13—C83 | 101.9(3) |
| C23—N13—Mg(1) | 119.0(2) |
| C83—N13—Mg(1) | 139.1(2) |
| N33—C23—N13 | 119.1(3) |
| N33—C23—S13 | 124.4(3) |
| N13—C23—S13 | 116.4(3) |
| C23—N33—C93 | 100.7(3) |
| C53—C43—C93 | 117.2(4) |
| C43—C53—C63 | 122.7(4) |
| C73—C63—C53 | 121.0(3) |
| C73—C63—O23 | 122.2(4) |
| C53—C63—O23 | 116.8(4) |
| C63—C73—C83 | 117.3(4) |
| N13—C83—C73 | 129.0(3) |
| N13—C83—C93 | 108.0(3) |
| C73—C83—C93 | 122.9(3) |
| N33—C93—C83 | 110.2(3) |
| N33—C93—C43 | 130.9(3) |
| C83—C93—C43 | 118.8(3) |
| C113—C103—S13 | 110.3(3) |
| N123—C113—C163 | 122.2(3) |
| N123—C113—C103 | 113.5(3) |
| C163—C113—C103 | 124.2(3) |
| C133—N123—C113 | 117.9(4) |
| N123—C133—C143 | 126.3(4) |
| C133—C143—C153 | 115.0(3) |
| C133—C143—C183 | 124.6(4) |
| C153—C143—C183 | 120.4(4) |
| O33—C153—C163 | 121.7(3) |
| O33—C153—C143 | 117.5(3) |
| C163—C153—C143 | 120.7(3) |
| C113—C163—C153 | 117.7(3) |
| C113—C163—C203 | 122.0(3) |
| C153—C163—C203 | 120.2(3) |
| S13—O13—Mg(1) | 120.42(19) |
| C63—O23—C173 | 117.7(5) |
| C153—O33—C193 | 117.5(5) |
| O13—S13—C23 | 102.82(18) |
| O13—S13—C103 | 105.47(19) |
| C23—S13—C103 | 98.2(2) |
| C24—N14—C84 | 101.8(3) |
| C24—N14—Mg(2) | 119.1(2) |
| C84—N14—Mg(2) | 139.0(3) |
| N34—C24—N14 | 119.4(3) |
| N34—C24—S14 | 124.2(3) |
| N14—C24—S14 | 116.5(3) |
| C24—N34—C94 | 100.6(3) |
| C54—C44—C94 | 117.2(4) |
| C44—C54—C64 | 122.2(4) |
| C74—C64—C54 | 121.9(4) |
| C74—C64—O24 | 123.5(4) |
| C54—C64—O24 | 114.5(4) |
| C64—C74—C84 | 116.9(4) |
| N14—C84—C74 | 129.8(4) |
| N14—C84—C94 | 107.8(3) |
| C74—C84—C94 | 122.4(3) |
| N34—C94—C84 | 110.5(3) |
| N34—C94—C44 | 130.1(4) |
| C84—C94—C44 | 119.4(3) |
| C114—C104—S14 | 111.7(3) |
| N124—C114—C164 | 122.8(3) |
| N124—C114—C104 | 114.4(3) |
| C164—C114—C104 | 122.8(3) |
| C734—N124—C114 | 117.6(4) |
| N124—C734—C144 | 125.6(4) |
| C734—C144—C154 | 115.4(3) |
| C734—C144—C184 | 123.5(4) |
| C154—C144—C184 | 121.1(4) |
| O34—C154—C164 | 120.2(3) |
| O34—C154—C144 | 119.0(3) |
| C164—C154—C144 | 120.8(3) |
| C154—C164—C114 | 117.7(3) |
| C154—C164—C204 | 119.9(3) |
| C114—C164—C204 | 122.4(3) |
| S14—O14—Mg(2) | 120.94(18) |
| C64—O24—C174 | 116.7(4) |
| C154—O34—C194 | 115.8(4) |
| O14—S14—C24 | 103.48(19) |
| O14—S14—C104 | 105.7(2) |
| C24—S14—C104 | 98.43(18) |
| C25—N15—C85 | 101.5(3) |
| C25—N15—Mg(2) | 116.4(2) |
| C85—N15—Mg(2) | 140.9(3) |
| N35—C25—N15 | 119.8(3) |
| N35—C25—S15 | 122.5(3) |
| N15—C25—S15 | 117.6(3) |
| C25—N35—C95 | 100.2(3) |
| C55—C45—C95 | 118.1(4) |
| C45—C55—C65 | 121.7(4) |
| C75—C65—O25 | 124.0(4) |
| C75—C65—C55 | 121.4(4) |
| O25—C65—C55 | 114.6(4) |
| C65—C75—C85 | 117.7(4) |
| N15—C85—C75 | 130.4(4) |
| N15—C85—C95 | 107.8(3) |
| C75—C85—C95 | 121.8(4) |
| N35—C95—C85 | 110.7(3) |
| N35—C95—C45 | 130.0(4) |
| C85—C95—C45 | 119.3(4) |
| C115—C105—S15 | 109.7(3) |
| N125—C115—C165 | 123.7(3) |
| N125—C115—C105 | 113.0(3) |
| C165—C115—C105 | 123.2(3) |
| C135—N125—C115 | 117.3(4) |
| N125—C135—C145 | 125.3(4) |
| C135—C145—C155 | 115.8(3) |
| C135—C145—C185 | 122.2(4) |
| C155—C145—C185 | 122.0(4) |
| O35—C155—C165 | 119.3(3) |
| O35—C155—C145 | 119.6(3) |
| C165—C155—C145 | 121.0(3) |
| C115—C165—C155 | 116.8(3) |
| C115—C165—C205 | 123.0(4) |
| C155—C165—C205 | 120.1(3) |
| S15—O15—Mg(2) | 120.79(18) |
| C65—O25—C175 | 116.3(4) |
| C155—O35—C195 | 114.9(4) |
| O15—S15—C25 | 102.72(18) |
| O15—S15—C105 | 105.7(2) |
| C25—S15—C105 | 97.6(2) |
| C26—N16—C86 | 102.1(3) |
| C26—N16—Mg(2) | 117.4(2) |
| C86—N16—Mg(2) | 139.7(3) |
| N36—C26—N16 | 119.4(3) |
| N36—C26—S16 | 123.8(3) |
| N16—C26—S16 | 116.8(3) |
| C26—N36—C96 | 100.5(3) |
| C56—C46—C96 | 118.0(3) |
| C46—C56—C66 | 121.9(3) |
| C76—C66—O26 | 123.0(4) |
| C56—C66—O26 | 115.6(4) |
| C66—C76—C86 | 118.0(4) |
| N16—C86—C76 | 130.6(3) |
| N16—C86—C96 | 107.5(3) |
| C76—C86—C96 | 121.8(3) |
| N36—C96—C86 | 110.6(3) |
| N36—C96—C46 | 130.3(4) |
| C86—C96—C46 | 119.1(3) |
| C116—C106—S16 | 112.8(3) |
| N126—C116—C166 | 122.7(3) |
| N126—C116—C106 | 113.7(3) |
| C166—C116—C106 | 123.5(3) |
| C136—N126—C116 | 117.6(4) |
| N126—C136—C146 | 125.5(4) |
| C136—C146—C156 | 115.5(3) |
| C136—C146—C186 | 123.0(4) |
| C156—C146—C186 | 121.5(4) |
| O36—C156—C166 | 120.6(3) |
| O36—C156—C146 | 118.4(3) |
| C166—C156—C146 | 120.8(3) |
| C156—C166—C116 | 117.6(3) |
| C156—C166—C206 | 120.6(4) |
| C116—C166—C206 | 121.7(4) |
| S16—O16—Mg(2) | 120.55(18) |
| C66—O26—C176 | 117.6(5) |
| C156—O36—C196 | 116.3(5) |
| O16—S16—C26 | 102.97(18) |
| O16—S16—C106 | 106.6(2) |
| C26—S16—C106 | 97.7(2) |
| O11—Mg(1)—O12 | 92.31(15) |
| O11—Mg(1)—N13 | 167.71(16) |
| O12—Mg(1)—N13 | 91.13(18) |
| O11—Mg(1)—O13 | 88.55(15) |
| O12—Mg(1)—O13 | 88.90(14) |
| N13—Mg(1)—O13 | 79.72(13) |
| O11—Mg(1)—N11 | 81.48(14) |
| O12—Mg(1)—N11 | 172.95(17) |
| N13—Mg(1)—N11 | 95.64(17) |
| O13—Mg(1)—N11 | 94.25(16) |
| O11—Mg(1)—N12 | 94.83(17) |
| O12—Mg(1)—N12 | 79.71(13) |
| N13—Mg(1)—N12 | 97.39(17) |
| O13—Mg(1)—N12 | 168.22(16) |
| N11—Mg(1)—N12 | 97.40(16) |
| O15—Mg(2)—O16 | 94.16(14) |
| O15—Mg(2)—N16 | 173.74(17) |
| O16—Mg(2)—N16 | 81.10(13) |
| O15—Mg(2)—N15 | 80.74(13) |
| O16—Mg(2)—N15 | 96.76(17) |
| N16—Mg(2)—N15 | 95.69(16) |
| O15—Mg(2)—O14 | 90.99(14) |
| O16—Mg(2)—O14 | 87.17(14) |
| N16—Mg(2)—O14 | 92.85(16) |
| N15—Mg(2)—O14 | 171.05(15) |
| O15—Mg(2)—N14 | 90.88(16) |
| O16—Mg(2)—N14 | 165.71(16) |
| N16—Mg(2)—N14 | 94.69(16) |
| N15—Mg(2)—N14 | 97.24(15) |
| O14—Mg(2)—N14 | 79.37(13) |
| O(5)—Mg(3)—O(6) | 84.2(2) |
| O(5)—Mg(3)—O(8) | 92.6(3) |
| O(6)—Mg(3)—O(8) | 99.2(2) |
| O(5)—Mg(3)—O(4) | 174.7(2) |
| O(6)—Mg(3)—O(4) | 90.6(2) |
| O(8)—Mg(3)—O(4) | 87.6(2) |
| O(5)—Mg(3)—O(9) | 90.9(3) |
| O(6)—Mg(3)—O(9) | 88.4(2) |
| O(8)—Mg(3)—O(9) | 171.9(3) |
| O(4)—Mg(3)—O(9) | 89.6(2) |
| O(5)—Mg(3)—O(7) | 94.0(2) |
| O(6)—Mg(3)—O(7) | 174.9(3) |
| O(8)—Mg(3)—O(7) | 85.6(2) |
| O(4)—Mg(3)—O(7) | 91.3(2) |
| O(9)—Mg(3)—O(7) | 86.9(2) |
| S(2)—O(7)—Mg(3) | 126.3(3) |
| O(7)—S(2)—C(31) | 104.0(4) |
| O(7)—S(2)—C(32) | 105.3(4) |
| C(31)—S(2)—C(32) | 95.8(3) |
| S(3)—O(8)—Mg(3) | 147.2(4) |
| O(8)—S(3)—C(33) | 102.5(4) |
| O(8)—S(3)—C(34) | 105.5(4) |
| C(33)—S(3)—C(34) | 95.8(4) |
| S(4)—O(9)—Mg(3) | 133.1(4) |
| O(9)—S(4)—C(35) | 103.0(4) |
| O(9)—S(4)—C(36) | 107.3(5) |
| C(35)—S(4)—C(36) | 94.5(4) |

### EXAMPLE 32

Evaluation of Magnesium S-Omeprazole Sample By X-Ray Powder Diffraction.

Magnesium S-omeprazole samples were placed on a zero background plate in a random orientation and evaluated by X-ray powder diffraction on a Siemens D500 using the following analysis parameters:
Range: 2.0-40.0° 2-theta scan
Slew: 2.4°/minute, continuous scan
Sampling rate: 0.05°/data point
Slits: 1°, 1°, 1°, 0.15°, 0.15°.
Radiation: CuK_{α}.
Power: 50 kV, 30 mA.

1. A magnesium S-omeprazolato coordination compound in the solid state according to formula (I):
[Mg(solvₐ)ₓ(solv_{b})_{y}][Mg(S-omeprazolato)₃]₂.(solv_{c})_{z} (I),
wherein
solvₐ is a solvent molecule that is selected from the group consisting of H₂O; ROH; RC(O)R; RC(O)OR; ROR; RC(S)R; RS(O)R; R₂NC(O)R; and an optionally substituted 5- or 6-membered heterocyclic compound comprising at least one heteroatom selected from the group consisting of O, S, and N;
solv_{b} is a solvent molecule that is selected from the group consisting of H₂O; ROH; RC(O)R; RC(O)OR; ROR; RC(S)R; RS(O)R; R₂NC(O)R; and an optionally substituted 5- or 6-membered heterocyclic compound comprising at least one heteroatom selected from the group consisting of O, S, and N;
solv_{c} represents at least one solvent molecule that is selected from the group consisting of H₂O; ROH; RC(O)R; RC(O)OR; ROR; RC(S)R; RS(O)R; R₂NC(O)R; and an optionally substituted 5- or 6-membered heterocyclic compound comprising at least one heteroatom selected from the group consisting of O, S, and N, wherein each solv_{c} can be the same as or different from another solv_{c};
wherein R, at each occurrence, is independently hydrogen or a C₁₋₆-alkyl group;
x and y, independently of each other, are selected from the integers 0-6 inclusive such that (x+y) is not more than 8;
z is a positive rational number from 0 to 6, inclusive; and
each S-omeprazolato ligand, independently of the others, is an anionic ligand of 5-methoxy-2-{[(4-methoxy-3,5-dimethylpyridin-2-yl)methyl]sulfinyl}-1H-benzimidazole or 6-methoxy-2-{[(4-methoxy-3,5-dimethylpyridin-2-yl)methyl]sulfinyl}-1H-benzimidazole.

2. The compound according to claim 1 wherein at least one S-omeprazolato ligand bears a 6-methoxy group.

3. The compound according to claim 2 wherein at least three S-omeprazolato ligands bear 6-methoxy groups.

4. The compound according to claim 3 wherein at least four S-omeprazolato ligands bear 6-methoxy groups.

5. The compound according to claim 4 wherein at least five S-omeprazolato ligands bear 6-methoxy groups.

6. The compound according to claim 5 wherein each S-omeprazolato ligand bears a 6-methoxy group.

7. The compound according to claim 1 wherein solvₐ, solv_{b} and solv_{c} are independently selected from the group consisting of H₂O, DMSO, DMF, acetone, and a C₁₋₆-alkyl alcohol.

8. The compound according to claim 7 wherein the C₁₋₆-alkyl-alcohol is methanol or ethanol.

9. The compound according to claim 7 wherein solvₐ, solv_{b} and solv_{c} are independently selected from DMF and H₂O.

10. The compound according to claim 9 wherein solvₐ is H₂O and solv_{b} and solv_{c} each are DMF.

11. The compound according to claim 7 wherein solvₐ, solv_{b} and solv_{c} are independently selected from DMSO and H₂O.

12. The compound according to claim 7 wherein at least one of solv₈, solv_{b} and solv_{c} is H₂O.

13. The compound according to claim 7 wherein at least one of solvₐ, solv_{b} and solv_{c} is DMSO.

14. The compound according to claim 7 wherein at least one of solvₐ, solv_{b} and solv_{c} is DMF.

15. The compound according to claim 7 wherein at least one Of solvₐ, solv_{b} and solv_{c} is acetone.

16. The compound according to claim 7 wherein at least one of solvₐ, solv_{b} and solv_{c} is methanol

17. The compound according to claim 7 wherein at least one of solvₐ, solv_{b} and solv_{c} is ethanol.

18. The compound according to claim 10 wherein x is 5 and y and z are each 1.

19. The compound according to claim 7 wherein solvₐ is H₂O and solv_{b} is DMSO.

20. The compound according to claim 19 wherein x and y are each 3 and z is 0.

21. The compound according to claim 1 that is:
Δ,Δ-[Mg(H₂O)₅DMF][Mg(6-methoxy-5-omeprazolato)₃][Mg(6-methoxy-S-omeprazolato)₂(5-methoxy-5-omeprazolato)].DMF;
Δ,Δ-[Mg(H₂O)₅DMF][Mg(6-methoxy-5-omeprazolato)₃][Mg(6-methoxy-S-omeprazolato)₂(5-methoxy-5-omeprazolato)].H₂O;
Δ,Δ-[Mg(H₂O)₅DMF][Mg(6-methoxy-5-omeprazolato)₃][Mg(6-methoxy-S-omeprazolato)₂(5-methoxy-5-omeprazolato)].(H₂O)_{z}(DMF)_{z}; or
mer-[Mg(H₂O)₃(DMSO)₃]-Δ,Δ-[Mg(5(6)-methoxy-5-omeprazolato)₃]₂.(H₂O)₃,
wherein S designates the absolute stereochemistry about each sulfur atom.

22. The compound according to claim 1, wherein at least one of solvₐ, solv_{b}, and solv_{c} represents H₂O and wherein x, y, and z are selected such that the ratio of total H₂O represented by at least one of solvₐ, solv_{b}, and solv_{c} to Mg ions in formula I ranges from about 1.5 to about 2.4.

23. A compound according to claim 1, characterized in that it exhibits the following major peaks in its powder X-ray diffractogram:

| d-value/Å | Relative Intensity |
|---|---|
| 15.3 | vs |
| 10.5 | s |
| 8.2 | s |
| 5.0 | s |
| 4.8 | vs |
| 4.0 | s |
| 3.7 | s |
| 2.9 | s |

24. A compound according to claim 1, characterized in that it exhibits the following major peaks in its powder X-ray diffractogram:

| d-value/Å | Relative Intensity |
|---|---|
| 15.5 | vs |
| 10.6 | m |
| 8.4 | s |
| 5.1 | vs |
| 4.8 | vs |
| 3.4 | s |
| 2.9 | s |

25. A compound according to claim 1, characterized in that it exhibits the following major peaks in its powder X-ray diffractogram:

| d-value/Å | Relative Intensity |
|---|---|
| 14.8 | vs |
| 12.2 | w |
| 10.8 | w |
| 8.4 | w |
| 7.6 | m |
| 6.7 | w |
| 5.5 | w |
| 5.1 | s |
| 4.8 | s |
| 4.3 | m |
| 4.1 | m |
| 3.8 | w |
| 3.5 | w |
| 2.9 | m |

26. A compound according to claim 1, characterized in that it exhibits the following major peaks in its powder X-ray diffractogram:

| d-value/Å | Relative Intensity |
|---|---|
| 15.1 | vs |
| 12.5 | m |
| 10.8 | m |
| 10.0 | m |
| 8.5 | m |
| 7.8 | m |
| 5.1 | vs |
| 4.8 | vs |
| 4.3 | m |
| 4.1 | m |
| 3.8 | m |
| 3.4 | m |
| 2.9 | m |

27. A compound according to claim 1, characterized in that it exhibits the following major peaks in its powder X-ray diffractogram:

| d-value/Å | Relative Intensity |
|---|---|
| 15.1 | vs |
| 12.4 | m |
| 10.9 | m |
| 8.5 | w |
| 7.8 | m |
| 6.9 | m |
| 5.5 | m |
| 5.1 | s |
| 5.0 | s |
| 4.8 | s |
| 4.8 | w |
| 4.3 | m |
| 4.1 | m |
| 3.9 | w |
| 3.7 | w |
| 3.5 | w |
| 3.5 | m |
| 3.4 | m |
| 2.9 | m |
| 2.5 | w |

28. A compound according to claim 1, characterized in that it exhibits the following major peaks in its powder X-ray diffractogram:

| d-value/Å | Relative Intensity |
|---|---|
| 15.1 | vs |
| 12.3 | m |
| 10.9 | m |
| 8.5 | m |
| 7.8 | m |
| 6.9 | m |
| 5.5 | m |
| 5.1 | s |
| 5.0 | s |
| 4.9 | vs |
| 4.7 | m |
| 4.3 | m |
| 4.1 | m |
| 3.9 | m |
| 3.7 | m |
| 3.5 | m |
| 3.4 | m |
| 3.4 | m |
| 2.9 | m |
| 2.5 | w |

29. A compound according to claim 1, characterized in that it exhibits the following major peaks in its powder X-ray diffractogram:

| d-value/Å | Relative Intensity |
|---|---|
| 14.9 | vs |
| 12.2 | m |
| 10.8 | m |
| 8.4 | m |
| 7.7 | m |
| 6.8 | m |
| 5.5 | m |
| 5.1 | s |
| 5.0 | s |
| 4.8 | vs |
| 4.6 | m |
| 4.4 | m |
| 4.3 | m |
| 4.1 | s |
| 4.0 | m |
| 3.9 | m |
| 3.8 | w |
| 3.7 | m |
| 3.5 | m |
| 3.4 | m |
| 2.9 | m |
| 2.5 | m |

30. A compound according to claim 1, characterized in that it exhibits the following major peaks in its powder X-ray diffractogram:

| d-value/Å | Relative Intensity |
|---|---|
| 14.9 | vs |
| 12.2 | m |
| 10.8 | m |
| 8.4 | m |
| 7.7 | m |
| 6.8 | m |
| 5.5 | s |
| 5.1 | s |
| 5.0 | s |
| 4.8 | vs |
| 4.6 | m |
| 4.4 | w |
| 4.2 | m |
| 4.1 | s |
| 3.9 | m |
| 3.8 | w |
| 3.7 | m |
| 3.5 | m |
| 3.4 | s |
| 2.9 | m |
| 2.5 | w |

31. A compound according to claim 1, characterized in that it exhibits the following major peaks in its powder X-ray diffractogram:

| d-value/Å | Relative Intensity |
|---|---|
| 19.0 | vs |
| 12.0 | m |
| 10.6 | vs |
| 9.2 | m |
| 7.3 | vs |
| 6.0 | m |
| 5.8 | m |
| 4.8 | vs |
| 4.4 | s |
| 4.1 | s |
| 3.5 | m |
| 3.3 | m |
| 2.9 | m |
| 2.8 | m |

32. A compound according to claim 1, characterized in that it exhibits the following major peaks in its powder X-ray diffractogram:

| d-value/Å | Relative Intensity |
|---|---|
| 18.6 | vs |
| 12.0 | s |
| 10.5 | vs |
| 7.8 | vs |
| 4.8 | vs |
| 4.8 | vs |
| 4.3 | s |
| 3.5 | s |

33. A compound according to claim 1, characterized in that it exhibits the following major peaks in its powder X-ray diffractogram:

| d-value/Å | Relative Intensity |
|---|---|
| 14.8 | vs |
| 12.1 | s |
| 10.7 | m |
| 8.4 | s |
| 7.8 | m |
| 6.7 | m |
| 5.7 | m |
| 5.4 | s |
| 5.0 | vs |
| 4.8 | vs |
| 4.7 | vs |
| 4.6 | s |
| 4.4 | m |
| 4.3 | s |
| 4.1 | s |
| 4.0 | w |
| 3.8 | w |
| 3.6 | m |
| 3.4 | m |
| 2.9 | m |
| 2.9 | m |
| 2.5 | w |

34. A compound according to claim 1, characterized in that it exhibits the following major peaks in its powder X-ray diffractogram:

| d-value/Å | Relative Intensity |
|---|---|
| 14.8 | vs |
| 12.2 | s |
| 10.7 | m |
| 8.4 | s |
| 7.7 | m |
| 7.3 | m |
| 6.7 | m |
| 5.7 | m |
| 5.5 | s |
| 5.3 | m |
| 5.0 | vs |
| 4.8 | vs |
| 4.7 | s |
| 4.6 | s |
| 4.4 | m |
| 4.3 | s |
| 4.1 | s |
| 4.0 | m |
| 3.8 | w |
| 3.7 | m |
| 3.4 | m |
| 2.9 | m |
| 2.9 | m |
| 2.5 | w |

35. A compound according to claim 1, characterized in that it exhibits the following major peaks in its powder X-ray diffractogram:

| d-value/Å | Relative Intensity |
|---|---|
| 17.3 | vs |
| 11.7 | m |
| 10.3 | s |
| 7.2 | vs |
| 6.3 | s |
| 4.8 | vs |
| 4.2 | s |
| 3.5 | s |
| 3.1 | m |

36. A compound according to claim 1, characterized in that it exhibits the following major peaks in its powder X-ray diffractogram:

| d-value/Å | Relative Intensity |
|---|---|
| 15.0 | vs |
| 12.2 | m |
| 7.7 | w |
| 7.1 | w |
| 6.5 | vs |
| 6.1 | vs |
| 5.8 | s |
| 5.2 | vs |
| 5.2 | s |
| 4.7 | vs |
| 4.5 | s |
| 4.3 | m |
| 4.2 | vs |
| 4.0 | s |
| 3.8 | m |
| 3.5 | m |
| 3.4 | m |
| 3.3 | m |
| 3.2 | s |
| 3.0 | s |
| 2.9 | m |
| 2.7 | w |
| 2.6 | m |
| 2.5 | w |
| 2.4 | m |
| 2.3 | m |

37. A compound according to claim 1, characterized in that it exhibits the following major peaks in its powder X-ray diffractogram:

| d-value/Å | Relative Intensity |
|---|---|
| 14.7 | vs |
| 12.2 | s |
| 10.6 | s |
| 8.5 | s |
| 7.8 | s |
| 7.3 | m |
| 6.7 | m |
| 5.7 | m |
| 5.4 | m |
| 5.3 | m |
| 5.1 | vs |
| 4.8 | vs |
| 4.7 | s |
| 4.6 | s |
| 4.4 | m |
| 4.3 | s |
| 4.1 | s |
| 4.0 | m |
| 3.8 | s |
| 3.7 | m |
| 3.4 | m |
| 2.9 | m |
| 2.9 | m |
| 2.5 | m |

38. A compound according to claim 1, characterized in that it exhibits the following major peaks in its powder X-ray diffractogram:

| d-value/Å | Relative Intensity |
|---|---|
| 15.0 | vs |
| 12.3 | s |
| 10.9 | s |
| 8.4 | s |
| 7.8 | m |
| 5.4 | s |
| 5.0 | vs |
| 4.8 | vs |
| 4.7 | vs |
| 4.1 | s |
| 3.4 | s |
| 2.9 | s |

39. A compound according to claim 1, characterized in that it exhibits the following major peaks in its powder X-ray diffractogram:

| d-value/Å | Relative Intensity |
|---|---|
| 17.0 | vs |
| 11.9 | m |
| 10.2 | s |
| 7.2 | m |
| 6.3 | m |
| 5.3 | s |
| 4.8 | vs |
| 4.2 | m |
| 4.0 | s |
| 3.5 | m |
| 3.2 | m |
| 2.7 | m |

40. A compound according to claim 1, characterized in that it exhibits the following major peaks in its powder X-ray diffractogram:

| d-value/Å | Relative Intensity |
|---|---|
| 19.4 | vs |
| 12.1 | m |
| 10.7 | vs |
| 9.2 | s |
| 7.3 | vs |
| 6.1 | s |
| 5.8 | s |
| 5.4 | s |
| 5.0 | vs |
| 4.8 | vs |
| 4.7 | s |
| 4.5 | s |
| 4.1 | s |
| 3.9 | s |
| 3.5 | s |
| 3.3 | s |
| 3.2 | m |
| 3.1 | m |
| 2.7 | m |

41. A compound according to claim 1, characterized in that it exhibits the following major peaks in its powder X-ray diffractogram:

| d-value/Å | Relative Intensity |
|---|---|
| 13.7 | vs |
| 9.4 | s |
| 8.7 | s |
| 8.0 | s |
| 7.2 | s |
| 6.2 | s |
| 5.8 | vs |
| 5.2 | vs |
| 5.0 | vs |
| 5.0 | vs |
| 4.9 | vs |
| 4.6 | vs |
| 4.6 | s |
| 4.5 | s |
| 4.4 | s |
| 4.3 | s |
| 4.1 | s |
| 4.0 | vs |
| 3.9 | s |
| 3.9 | vs |
| 3.7 | vs |
| 3.6 | vs |
| 3.5 | vs |
| 3.4 | s |
| 3.3 | s |
| 3.1 | s |
| 3.0 | s |

42. A compound according to claim 1, characterized in that it exhibits the following major resonances in its solid-state ¹³C NMR spectrum:
Chemical Shift
(δ) (ppm)
166.6
165.9
164.6
162.6
158.5
157.4
155.5
150.2
148.7
143.9
142.5
138.8
129.8
128.7
127.7
118.4
115.9
112.0
110.4
99.3
97.0
62.5
60.0
59.1
55.1
53.6
35.3
30.9
11.7
11.0
9.6

43. A compound according to claim 1, characterized in that it exhibits the following major resonances in its solid-state ¹³C NMR spectrum:
Chemical Shift
(δ) (ppm)
167.1
166.6
164.5
156.8
155.9
149.2
144.0
141.6
138.8
129.9
128.7
127.2
118.6
117.5
116.0
112.3
111.4
97.8
96.4
62.5
59.4
56.0
54.6
53.1
30.1
13.2
11.8
10.0

44. A compound according to claim 1, characterized in that it exhibits the following major resonances in its solid-state ¹³C NMR spectrum:
Chemical Shift
(δ) (ppm)
164.0
154.6
149.8
146.6
142.6
140.2
136.8
126.3
116.2
111.5
95.9
58.1
52.5
10.5
7.8

45. A pharmaceutical composition comprising a therapeutically effective amount of a compound according to claim 1 and a pharmaceutically acceptable carrier, diluent, excipient, or combination thereof.

46. A method of treating a gastric acid related condition in a subject comprising administering to the subject suffering from the condition a therapeutically effective amount of the compound according to claim 1.

47. A method of treating a gastric acid related condition in a subject comprising administering to the subject suffering from the condition a therapeutically effective amount of the pharmaceutical composition according to claim 45.

48. A method of inhibiting gastric acid secretion in a subject comprising administering to the subject suffering from the condition a therapeutically effective amount of the compound according to claim 1.

49. A method of inhibiting gastric acid secretion in a subject comprising administering to the subject suffering from the condition a therapeutically effective amount of pharmaceutical composition according to claim 45.

50. A magnesium R-omeprazolato coordination compound in the solid state according to formula (II):
[Mg(solvₐ)ₓ(solv_{b})_{y}][Mg(R-omeprazolato)₃]₂.(solv_{c})_{z} (II),
wherein
solvₐ is a solvent molecule that is selected from the group consisting of H₂O; ROH; RC(O)R; RC(O)OR; ROR; RC(S)R; RS(O)R; R₂NC(O)R; and an optionally substituted 5- or 6-membered heterocyclic compound comprising at least one heteroatom selected from the group consisting of O, S, and N;
solv_{b} is a solvent molecule that is selected from the group consisting of H₂O; ROH; RC(O)R; RC(O)OR; ROR; RC(S)R; RS(O)R; R₂NC(O)R; and an optionally substituted 5- or 6-membered heterocyclic compound comprising at least one heteroatom selected from the group consisting of O, S, and N;
solv_{c} represents at least one solvent molecule that is selected from the group consisting of H₂O; ROH; RC(O)R; RC(O)OR; ROR; RC(S)R; RS(O)R; R₂NC(O)R; and an optionally substituted 5- or 6-membered heterocyclic compound comprising at least one heteroatom selected from the group consisting of O, S, and N, wherein each solv_{c} can be the same as or different from another solv_{c};
wherein R, at each occurrence, is independently hydrogen or a C₁₋₆-alkyl group;
x and y, independently of each other, are selected from the integers 0-6 inclusive such that (x+y) is not more than 8;
z is a positive rational number from 0 to 6, inclusive; and
each R-omeprazolato ligand, independently of the others, is an anionic ligand of 5-methoxy-2-{[(4-methoxy-3,5-dimethylpyridin-2-yl)methyl]sulfinyl}-1H-benzimidazole or 6-methoxy-2-{[(4-methoxy-3,5-dimethylpyridin-2-yl)methyl]sulfinyl}-1H-benzimidazole.

51. A magnesium omeprazolato coordination compound in the solid state according to formula (IIIa):
[Mg(solvₐ)ₓ(solv_{b})_{y}][Mg(omeprazolato)₃]₂.(solv_{c})_{z} (IIIa),
wherein
solvₐ is a solvent molecule that is selected from the group consisting of H₂O; ROH; RC(O)R; RC(O)OR; ROR; RC(S)R; RS(O)R; R₂NC(O)R; and an optionally substituted 5- or 6-membered heterocyclic compound comprising at least one heteroatom selected from the group consisting of O, S, and N;
solv_{b} is a solvent molecule that is selected from the group consisting of H₂O; ROH; RC(O)R; RC(O)OR; ROR; RC(S)R; RS(O)R; R₂NC(O)R; and an optionally substituted 5- or 6-membered heterocyclic compound comprising at least one heteroatom selected from the group consisting of O, S, and N;
solv_{c} represents at least one solvent molecule that is selected from the group consisting of H₂O; ROH; RC(O)R; RC(O)OR; ROR; RC(S)R; RS(O)R; R₂NC(O)R; and an optionally substituted 5- or 6-membered heterocyclic compound comprising at least one heteroatom selected from the group consisting of O, S, and N, wherein each solv_{c} can be the same as or different from another solv_{c};
wherein R, at each occurrence, is independently hydrogen or a C₁₋₆-alkyl group;
x and y, independently of each other, are selected from the integers 0-6 inclusive such that (x+y) is not more than 8;
z is a positive rational number from 0 to 6, inclusive;
each omeprazolato ligand, independently of the others, is an anionic ligand of 5-methoxy-2-{[(4-methoxy-3,5-dimethylpyridin-2-yl)methyl]sulfinyl}-1H-benzimidazole or 6-methoxy-2-{[(4-methoxy-3,5-dimethylpyridin-2-yl)methyl]sulfinyl}-1H-benzimidazole; and
there exists an enantiomeric excess of S-omeprazolato ligands over R-omeprazolato ligands.

52. A magnesium omeprazolato coordination compound in the solid state according to formula (IIIb):
[Mg(solvₐ)ₓ(solv_{b})_{y}][Mg(omeprazolato)₃]₂.(solv_{c})_{z} (IIIb),
wherein
solvₐ is a solvent molecule that is selected from the group consisting of H₂O; ROH; RC(O)R; RC(O)OR; ROR; RC(S)R; RS(O)R; R₂NC(O)R; and an optionally substituted 5- or 6-membered heterocyclic compound comprising at least one heteroatom selected from the group consisting of O, S, and N;
solv_{b} is a solvent molecule that is selected from the group consisting of H₂O; ROH; RC(O)R; RC(O)OR; ROR; RC(S)R; RS(O)R; R₂NC(O)R; and an optionally substituted 5- or 6-membered heterocyclic compound comprising at least one heteroatom selected from the group consisting of O, S, and N;
solv_{c} represents at least one solvent molecule that is selected from the group consisting of H₂O; ROH; RC(O)R; RC(O)OR; ROR; RC(S)R; RS(O)R; R₂NC(O)R; and an optionally substituted 5- or 6-membered heterocyclic compound comprising at least one heteroatom selected from the group consisting of O, S, and N, wherein each solv_{c} can be the same as or different from another solv_{c};
wherein R, at each occurrence, is independently hydrogen or a C₁₋₆-alkyl group;
x and y, independently of each other, are selected from the integers 0-6 inclusive such that (x+y) is not more than 8;
z is a positive rational number from 0 to 6, inclusive;
each omeprazolato ligand, independently of the others, is an anionic ligand of 5-methoxy-2-{[(4-methoxy-3,5-dimethylpyridin-2-yl)methyl]sulfinyl}-1H-benzimidazole or 6-methoxy-2-{[(4-methoxy-3,5-dimethylpyridin-2-yl)methyl]sulfinyl}-1H-benzimidazole; and
there exists an enantiomeric excess of R-omeprazolato ligands over S-omeprazolato ligands.

53. A magnesium omeprazolato coordination compound in the solid state according to formula IIIc:
[Mg(solvₐ)ₓ(solv_{b})_{y}][Mg(omeprazolato)₃]₂.(solv_{c})_{z} (IIIc),
wherein
solvₐ is a solvent molecule that is selected from the group consisting of H₂O; ROH; RC(O)R; RC(O)OR; ROR; RC(S)R; RS(O)R; R₂NC(O)R; and an optionally substituted 5- or 6-membered heterocyclic compound comprising at least one heteroatom selected from the group consisting of O, S, and N;
solv_{b} is a solvent molecule that is selected from the group consisting of H₂O; ROH; RC(O)R; RC(O)OR; ROR; RC(S)R; RS(O)R; R₂NC(O)R; and an optionally substituted 5- or 6-membered heterocyclic compound comprising at least one heteroatom selected from the group consisting of O, S, and N;
solv_{c} represents at least one solvent molecule that is selected from the group consisting of H₂O; ROH; RC(O)R; RC(O)OR; ROR; RC(S)R; RS(O)R; R₂NC(O)R; and an optionally substituted 5- or 6-membered heterocyclic compound comprising at least one heteroatom selected from the group consisting of O, S, and N, wherein each solv_{c} can be the same as or different from another solv_{c};
wherein R, at each occurrence, is independently hydrogen or a C₁₋₆-alkyl group;
x and y, independently of each other, are selected from the integers 0-6 inclusive such that (x+y) is not more than 8;
z is a positive rational number from 0 to 6, inclusive;
each omeprazolato ligand, independently of the others, is an anionic ligand of 5-methoxy-2-{[(4-methoxy-3,5-dimethylpyridin-2-yl)methyl]sulfinyl}-1H-benzimidazole or 6-methoxy-2-{[(4-methoxy-3,5-dimethylpyridin-2-yl)methyl]sulfinyl}-1H-benzimidazole; and
there exists an equal number of R-omeprazolato and S-omeprazolato ligands.

54. A magnesium omeprazolato coordination compound in the solid state according to formula IV:
[Mg(omeprazolato)₃]X (IV),
wherein X is a monovalent cation.
